(19) United States
(12) Patent Application Publication
(10) Pub. No.: US 2026/0256716 A1
(43) Pub. Date: Sep. 3, 2026

(54) COMPOSITIONS AND METHODS OF USE THEREOF

(71) Applicant: Board of Supervisors of Louisiana State University and Agricultural and Mechanical College, Baton Rouge, LA (US)

(72) Inventors: Timothy Paul FOSTER, Slidell, LA (US); Thomas E. GALBATO

(21) Appl. No.: 19/163,132

(22) PCT Filed: Mar. 11, 2024
(86) PCT No.: PCT/US2024/019401

§ 371 Date: Sep. 8, 2025

Related U.S. Application Data
(60) Provisional application No. 63/451,447, filed on Mar. 10, 2023.
(60) Provisional application No. 63/612,800, filed on Dec. 20, 2023.

(51) Int. Cl. A61K 31/137; A61K 31/135; A61P 27/02

(52) U.S. Cl. CPC A61K 31/137; A61K 31/135; A61P 27/02

(57) ABSTRACT

The disclosure is drawn towards compositions and methods for treating and preventing senescence, mitochondrial dysfunction, and related diseases and disorders.

This application claims priority from U.S. Provisional Application No. 63/451,447 filed on Mar. 10, 2023, and U.S. Provisional Application No. 63/612,800 filed on Dec. 20, 2023, the entire contents of each of which are incorporated herein by reference.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

The present invention relates to compositions and methods for treating senescence, mitochondrial dysfunction, and related disease and disorders.

### BACKGROUND OF THE INVENTION

Serotonin receptors have not been associated with aging associated diseases. As such, identifying the role of this receptor in these processes provides a unique and new target.

### SUMMARY OF THE INVENTION

Aspects of the invention are drawn towards a method of treating or preventing senescence, mitochondrial dysfunction, or a disease or disorder thereof, the method comprising administering a therapeutically effective amount of a 5-HT_{2A} receptor agonist to a subject in need thereof, wherein the 5-HT_{2A} receptor agonist is selected from the group consisting of

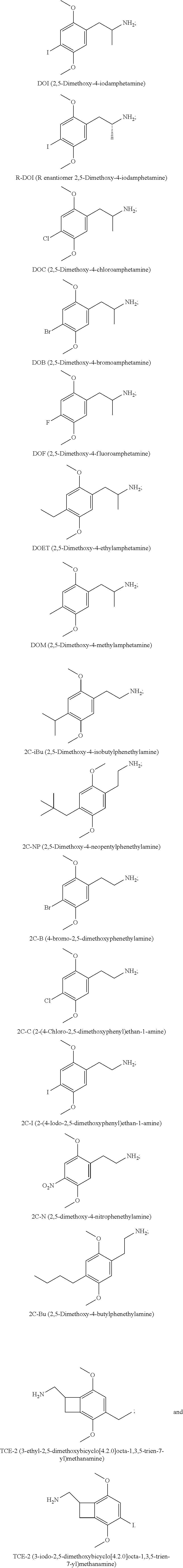

In embodiments, the disease or disorder comprises a neurodegenerative disease, a metabolic disorder, an inflammatory or autoimmune disease, or an aging related disorder. In embodiments, the neurodegenerative disease comprises dementia, Alzheimer's disease, Parkinson's disease, Huntington's disease, dementia, cognitive impairment, and motor neuron dysfunction. In embodiments, the metabolic disorder comprises type II diabetes, obesity, hyperglycemia, glucose intolerance, insulin resistance (i.e., hyperinsulinemia, metabolic syndrome, syndrome X), hypercholesterolemia, hypertension, hyperlipoproteinemia, hyperlipidemia (e.g., dyslipidemia), hypertriglylceridemia, cardiovascular disease, atherosclerosis, peripheral vascular disease, kidney disease, ketoacidosis, thrombotic disorders, nephropathy, diabetic neuropathy, diabetic retinopathy, sexual dysfunction, dermatopathy, dyspepsia, hypoglycemia, cancer or edema. In further embodiments, the inflammatory or autoimmune disease comprises celiac disease, vasculitis, lupus, chronic obstructive pulmonary disease (COPD), atherosclerosis, arthritis, psoriasis, atopic dermatitis, vitiligo, keratitis, allergic conjunctivitis, uveitis, retinitis, osteoarthritis, rheumatoid arthritis, osteoporosis, oral mucositis, inflammatory bowel disease, kyphosis, and herniated intervertebral disc. In embodiments, the aging related disorder comprises renal disease, renal failure, frailty, hearing loss, muscle fatigue, skin conditions, skin wound healing, liver fibrosis, pancreatic fibrosis, oral submucosa fibrosis, sarcopenia, cancer, dementia, cardiovascular disease, such as atherosclerosis, hypertension, diabetes mellitus (type I or type II) arthritis, dermatitis, cataracts, Alzheimer's disease, Parkinson's disease, diabetic retinopathies, retinopathies, glaucoma, age-related macular degeneration (AMD), and osteoporosis.

In embodiments, the therapeutically effective amount of a compound described herein comprises greater than about 1 uM, about 1 uM, less than about 1 uM, about 100 nM, about 10 nM, about 1 nM, about 0.1 nM, about 0.05 nM, about 0.001, about 0.0001 nM, about 0.00001 nM, and less than 0.00001 nM. In embodiments, the therapeutically effective amount of R-DOI is about 10 nM to less about 0.001 nM. In embodiments, the therapeutically effective amount of TCE-2 is about 100 nM. In embodiments, the therapeutically effective amount of TCI-2 is about 1 uM, less than about 1 uM, about 100 nM, about 10 nM, about 1 nM, about 0.1 nM, about 0.05 nM, about 0.001, and less than 0.001 nM. In embodiments, the therapeutically effective amount of 2C-NP is about 0.1 nM, about 0.01 nM, about 0.001 nM, about 0.0001 nM, or less than 0.1 nM. In embodiments, the therapeutically effective amount of 2C-B, 2C-C, 2C-I, 2C-Bu, or 2C-N is about 0.1 nM, about 0.001 nM, about 0.0001 nM, or less than 0.1 nM. In embodiments, the therapeutically effective amount of DOI is about 10 uM to about 50 uM.

In embodiments, the 5-HT_{2A} receptor agonist is formulated as a pharmaceutical composition comprising one or more pharmaceutically acceptable excipients. In embodiments, the pharmaceutical composition is formulated for topical administration, ocular administration, parenteral administration, intravenous administration, intranasal administration, or oral administration. In embodiments, the pharmaceutical composition is formulated for ocular administration can comprise an eye drop, a contact lens, an ocular hydrogel, an intraocular implant, or intravitreal injection.

Aspects of the invention are drawn towards a method of identifying a subject responsive to a 5-HT_{2A} receptor agonist of claim 1 for the treatment or prevention of senescence, mitochondrial dysfunction, or a disease or disorder thereof, the method comprising determining if the subject meets one or more of the following criteria: at or above the transition age of ocular disease development; a blood glucose level greater than or equal to about 6 mmol/L; presents with lipofuscin and drusen deposition; presents with autofluorescent oxidized flavoproteins; an Early Treatment Diabetic Retinopathy Study (ETDRS) disease severity score of about 20 or greater; and/or presents with a retinopathy. In embodiments, the transition age of ocular disease development comprises about 50 years old to about 60 years old in a human subject.

Other objects and advantages of this invention will become readily apparent from the ensuing description.

### BRIEF DESCRIPTION OF THE FIGURES

FIG. 1 shows exemplary data. Western blot analysis demonstrates that various cells isolated from retinal pigmented epithelia express 5-HT2A target receptors.

FIG. 2 shows exemplary data. Specific quantitative detection of 5-HT2A receptor expression in ARPE-19 retinal pigmented epithelial cells was optimized by flow cytometric analysis with optimal primary antibody concentrations determined to be 0.5 ug/ml to 1 ug/ml.

FIG. 3 shows non-limiting, exemplary data of ARPE-19 cells specifying 5-HT2A receptor knockouts. Levels of Serotonin 2A receptor expression were quantified by densitometry of western bands and normalized to GAPDH loading controls. Values of expression relative to ARPE-19 wildtype set at 100% were determined for each of the serotonin 2A receptor knockout cell-lines generated. Significance was assessed by one way ANOVA.

FIG. 4 shows non-limiting, exemplary data. Panel A shows fluorescent microscopy. Panel B shows a graph of fold increase in cell surface area. Panel C shows a graph of trypsinized cell area. Panel D shows graphs of cell size and dead/degranulated cells. Panel E shows an image of cells (Tarau et al., int. J. Mol. Sci. 2019, 20(14), 3578).

FIG. 5 shows non-limiting, exemplary images of ARPE cells.

FIG. 6 shows non-limiting, representative data. A Hallmark of Age-Associated Ocular and Neurodegenerative Disorders is Cytoskeletal Alteration and Induction of CONTRACTILE Stress Fiber Formation. Expression and activation via phosphorylation of Myosin Light Chain (MLC), is a biomarker of disease development. ARPE-19 cells and ARPE-19 cells specifying 5-HT2AR knockouts were plated in nutrient rich media (DMEM; 7% FCS) overnight. Cells were fixed, permeabilized, and stained phalloidin and α-phosphorylated-Myosin light chain. Cytoskeletal elements were then visualized by fluorescent microscopy to visualize actin fiber cytoskeletal structures (phalloidin, red) and contractile phosphorylated myosin light chain (green) via fluorescent microscopy. Representative western blot demonstrating that Low Dose Serotonin 2A Receptor Agonists, R-DOI and 2CIBu inhibit Hyperglycemic-Mediated MLC Activation and thereby Suppress Cytoskeletal Dysfunction associated with disease development. ARPE-19 cells were cultured under hyperglycemic conditions in nutrient rich media (DMEM High glucose; 7% FCS) overnight either in the absence or presence of 5-HT2A receptor agonists R-DOI and 2CiBu at 10 nm or 100 nM concentrations. The relative expression of the activated disease biomarker contractile phosphorylated myosin light chain was determined using specific antibodies and western blot analysis. 5-HT2A agonists. R-DOI and 2C-iBu suppressed hyperglycemia induced phosphorylated MLC levels at 10 nM concentrations. Therefore, 5-HT2A agonism at low concentrations have the potential to prevent and reverse deleterious cytoskeletal events that can be induced under hyperglycemic conditions, such as in diabetics.

FIG. 7 shows non-limiting, exemplary graphs of growth curves and proliferation.

FIG. 8 shows non-limiting, exemplary data. ARPE-19 cells specifying knockouts for 5-HT2A receptor expression exhibit altered cell cycle progression indicative of changes in RPE cell health and replicative capacity. Compared to ARPE-19 controls, ARPE-19 cells specifying 5-HT2A receptor knockouts exhibit a higher percentage of cells in G1 arrest and a lower percentage of cells in S phase of the cell cycle.

FIG. 9 shows exemplary graphs. Panel A shows 5-HT2A Receptor Agonists Increase Mitochondrial Function And ATP Production. Panel B shows 5-HT2A Receptor KNOCKDOWNS DECREASE Mitochondrial Function And ATP Production

FIG. 10 shows non-limiting, exemplary ARPE cell data.

FIG. 11 shows non-limiting, exemplary data. ARPE-19 cells specifying knockouts for 5-HT2A receptor expression exhibit increased disease promoting inflammatory cytokine production, consistent with development of retinopathies and AMD. VEGF and IL-6 inflammatory cytokine production and secretion was assessed via ELISA.

FIG. 12 shows an illustration of hallmarks of that can contribute to the development of age-related diseases (adapted from Schmauck-Medina et al., Aging (Albany NY). 2022 Aug. 29; 14:6829-6839. doi.org/10.18632/aging.204248).

FIG. 13 shows a nonlimiting diagrammatic summary of the underlying pathophysiological events that are associated with development of age-related ocular and neurodegenerative disease processes. The pathophysiology of Age-Related Diseases, including AMD, retinopathies, and neurodegenerative diseases, include cytoskeletal and cell size alterations, Lysosomal dysfunction, Autophagic Dysfunction of Toxic Proteins and Lipids, Metabolic Dysfunction, Oxidative Stress, and a Cell Senescent Phenotype. In AMD, these disease processes disrupt the retinal pigmented epithelial (RPE) layer as a patient ages. (Tarau et al., Int. J. Mol. Sci. 2019, 20(14), 3578).

FIG. 14 shows images of 5-HT2A receptors (doi: 10.1089/jp.2017.0125) and an exemplary Western blot.

FIG. 15 shows exemplary graphs of retrospective analysis of RNAseq data from clinically derived RPE.

FIG. 16 shows an exemplary graph plotting 5-HT2A Receptor Expression and age.

FIG. 17 shows an image of healthy RPE cells (doi: 10.1007/s00018-021-03796-9) and an exemplary graph of ARPE-19 replication.

FIG. 18 shows exemplary graphs. Specific 5-HT2A Receptor Activation via Model Phenethylamines, like R-DOI. Stimulate and Restore Healthy Proliferation of RPE at VERY Low CONCENTRATIONS between 0.1 to 1 nM. ARPE-19 cells were plated in nutrient rich media, DMEM, containing 7% dialyzed FCS and then starved in DMEM media containing 0.5% dialyzed FCS. Cells were then changed to 7% dialyzed FCS DMEM media containing physiolocial relevant levels of serotonin at varying concentrations and grown for 72-hours. Cell numbers were determined through DNA staining with DAPI and fold change in growth was determined from baseline readings at 24-hours. To assess if agonists of the 5HT2A receptor could enhance cell health and proliferation specifically through the 5-HT2A receptor, the model 5-HT2A receptor agonist, R-DOI was added at either 0.1 nM or 1 nM. Very low concentration 5-HT2A receptor agonism positively increased cellular function and replication, indicating that receptor agonism could be used to restore cellular function lost with age at low dosages or at increased dosages in aged patients experiencing disease-inducing decreases in 5-HT2A receptor expression levels. Also, included is a summary of physiological serotonin and 5-HT2A receptor agonist/antagonist outcomes on cellular proliferation. This increase in proliferation promotes the formation of a compact RPE-monolayer which is important for maintaining a healthy blood-retinal barrier and can occur with low dose administration of 5-HT2A receptor agonists.

FIG. 19 shows exemplary experimental data. The pathophysiological impact of the Age-Associated Decline in Serotonin 2A Receptor Expression was assessed by Generating CRISPR/Cas9 Site-Directed Knockout of the Serotonin 2A Receptor in RPE cells. A CRISPR/Cas9 system was utilized for site-directed knockout of the serotonin 2A receptor in ARPE-19 cells. Purification of cell-lines specifying either single or double allele knockouts of 5-HT2AR were achieved through FACS sorting. Assessment of levels of 5-HT2A Receptor expression was determined by western blot analysis. Cell lysates from each isolated cell line were collected and normalized to protein content prior to running western blot analysis specific for either serotonin 2A receptor or cellular GAPDH protein expression. Levels of Serotonin 2A receptor expression was quantified by densitometry of western bands on collected images, normalized to GAPDH. Values of expression relative to ARPE-19 wildtype set at 100% were determined for each of the serotonin 2A receptor knockout cell-lines generated.

FIG. 20 shows a representative growth curve. In the absence of the 5-HT2A receptor, cell health and proliferation are reduced. This is consistent with the Age-Related Declines in Repair of the Blood-Retinal Barrier in AMD, Diabetic Retinopathy, and Other Retinopathies. ARPE-19 cells and ARPE-19 cells specifying 5-HT2AR knockout/knockdowns were plated in nutrient rich media (DMEM; 7% FCS) and grown for 5-days. Cell numbers were determined through DNA staining with DAPI and fold change in growth was determined from baseline readings at 24-hours (Day 1). The knockdown/knock out of 5-HT2A receptor mimics the effects observed in patient samples that exhibit a decline in 5-HT2A receptor expression and function observed in aging individuals.

FIG. 21 shows exemplary images of healthy RPE and processes that disrupt its function. In AMD, cellular alteration in RPE size, morphology, & cytoskeletal network disrupt cellular function, barrier integrity and result in disease progression. These aspects increase as individuals age and correlates with decreased 5-HT2A receptor expression and function. Healthy RPE are normally a single layer of highly-organized, polygonal cells with similar size and shape, maintaining a healthy blood-retinal barrier. The cytoskeletal network in the RPE is important for maintaining a healthy state of elasticity and permeability. Cytoskeletal elements influence cell size, morphology, subcellular trafficking, and multi-cellular communication. Cytoskeletal dysregulation impacts RPE integrity and is a well-documented feature of retinopathies, including AMD, as well as neurodegenerative disorders.

FIG. 22 shows a non-limiting, representative experimental data. Age-Related Decreases in 5-HT2A Receptor expression and function Correlate with Pathological Development of Age-associated Ocular Disease. Consistent with Irregular Morphology and the Disrupted RPE in AMD, Diabetic Retinopathy, and Other Retinopathies, Serotonin 2A Receptor Knockouts have Increased Cell Surface Area Compared to the ARPE-19 Wildtype when trypsinized to single non-adherent cells. ARPE-19 cells and ARPE-19 cells specifying 5-HT2AR knockout/knockdowns were plated in nutrient rich media (DMEM: 7% FCS) overnight. Cells were trypsinized, nuclei stained with DAPI and imaged via microscopy or assessed via flow cytometry forward scatter analysis for relative size differences. Cell area was determined by analyzing image regions denoted by the cell areas observed via phase contrast imaging. Cell surface area was determined and calculated through ImageJ pixel content analysis. Decreases in 5-HT2A receptor expression and function are associated with hallmark pathologies associated with age-related disease development, including increases in cell size/morphology that disrupts barrier integrity and cellular functions.

FIG. 23 shows non-limiting, representative experimental data. Age-Related Decreases in 5-HT2A Receptor expression and function Correlate with Pathological Development of Age-associated Ocular Disease. Consistent with Irregular Morphology and the Disrupted RPE in AMD. Diabetic Retinopathy, and Other Retinopathies, Serotonin 2A Receptor Knockouts have Increased Cell Surface Area Compared to the ARPE-19 Wildtype. ARPE-19 cells and ARPE-19 cells specifying 5-HT2AR knockout/knockdowns were plated in nutrient rich media (DMEM; 7% FCS) overnight. Cells were stained with a cell surface dye and nuclei were stained with DAPI. Images were acquired via fluorescent microscopy. Cell area was determined by analyzing image regions denoted by the fluorescently stained green cell areas. Cell surface area was determined and calculated through ImageJ pixel content analysis. Decreases in 5-HT2A receptor expression and function are associated with hallmark pathologies associated with age-related disease development, including increases in cell size/morphology that disrupts barrier integrity and cellular functions.

FIG. 24 shows non-limiting exemplary data. RPE cells knockdowned/knockout for 5-HT2A receptor expression and function exhibit high levels of stress fiber formation (red), as well as association of phosphorylated MLC (green) on stress fibers that would be associated with disease development.

FIG. 25 shows a representative schematic. Described herein are data that assesses the effects of aging-associated decreases in 5-HT2A receptor Expression and Activity Results in Disease-inducing Defects in Lysosomal Content and Trafficking, Toxic Protein Processing and metabolic Dysregulation Characteristic of Aging-Associated Disease Processes. Previously identified cytoskeletal rearrangement affects phagocytosis & intracellular trafficking of potentially toxic cargo destined for degradation. A major biological function of the RPE is to phagocytose cellular debris and photoreceptor outer segments in the retina and subject it to lysosomal degradation. Dysregulation of the autophagy-lysosomal system is well-described in neurodegenerative disorders, including AMD. The inability to efficiently degrade protein/lipids results in the buildup of auto-fluorescent deposits in the RPE, termed drusen bodies. The presence of autofluorescence is a biomarker of disease development.

FIG. 26 shows a schematic of defects in RPE mediated protein and lipid processing, lysosomal trafficking and autophagy. Defects in these pathways culminate in drusen body formation and deposition that is a characteristic hallmark of ocular diseases like AMD and neurodegenerative diseases.

FIG. 27 shows non-limiting, exemplary data. Decreased 5-HT2A receptor Expression and Activity Results in Disease-inducing Defects in Lysosomal Content and Trafficking, Toxic Protein Processing AND METABOLIC Dysregulation Characteristic of Aging-Associated Disease Processes. Consistent with Morphological Abnormalities, a Disrupted RPE Monolayer, and Lipofuscin/Drusen Accumulation in AMD, Diabetic Retinopathy, and Other Retinopathies, Serotonin 2A Receptor Knockouts have Increased Granularity/Complexity Phenotype. Dysregulation of the autophagy-lysosomal system is well-described in neurodegenerative disorders, including AMD. The inability to efficiently degrade protein/lipids results in the buildup of auto-fluorescent deposits in the RPE, termed drusen bodies. Increased granularity can indicate an over accumulation of lysosomal content that has failed to clear deleterious toxic proteins and lipids from the environment. ARPE-19 cells and ARPE-19 cells specifying 5-HT2AR knockout were plated in nutrient rich media (DMEM; 7% FCS) overnight. Cells were then suspended to analyze granularity/complexity via flow cytometry. 5-HT2A knockouts exhibited increased granularity indicative of lysosomal dysfunction.

FIG. 28 shows non-limiting, exemplary data. Decreased 5-HT2A receptor Expression and Activity Results in Disease-inducing Defects in Lysosomal Content and Trafficking, Toxic Protein Processing and Metabolic Dysregulation Characteristic of Aging-Associated Disease Processes. Consistent with Lysosomal Dysfunction and Inadequate Waste Degradation in AMD, Diabetic Retinopathy, and Other Retinopathies, Serotonin 2A Receptor Knockouts Have Increased Lysosomal Biomass Compared to the ARPE-19 Wildtype. Dysregulation of the autophagy-lysosomal system is well-described in neurodegenerative disorders, including AMD. The inability to efficiently degrade protein/lipids results in the buildup of auto-fluorescent deposits in the RPE, termed drusen bodies. Increased lysosomal content can indicate an abundance and failure to clear deleterious toxic proteins and lipids from the environment. ARPE-19 cells and ARPE-19 cells specifying 5-HT2AR knockout were plated in nutrient rich media (DMEM; 7% FCS) overnight. Cells were stained for lysosomal content using lysotracker dyes, suspended, and analyzed via flow cytometry for relative lysosomal content by determining mean fluorescent intensity per cell. 5-HT2A knockouts exhibited increased lysosomal content in congruence with the pathophysiological processes associated with ocular and neurogenerative diseases of aging. Lysosomal dysfunction is recognized as a factor for age-related and neurodegenerative diseases.

FIG. 29 shows non-limiting, exemplary data. Relative levels of autofluorescent lipofuscin accumulation was assessed by flow cytometric analysis in control ARPE-19 cells, APRE-19 cells treated with disease inducing etoposide, or ARPE-19 cells specifying 5-HT2A receptor knockouts. Decreased 5-HT2A receptor function was associated with increased auto fluorescent lipofuscin production, consistent with development of drusen in AMD, a biomarker of the extent of disease development.

FIG. 30 shows a non-limiting schematic of the influence of aging, environmental, smoking and UV/blue light exposure on development of disease processes that underlie development of retinopathies and neurodegenerative diseases (doi.org/10.3389/f phar.2018 0.01280). These factors induce lipofuscin accumulation, mitochondrial dysfunction and increases in reactive oxygen species. RPE cells can inherently counter these insults through the induction of the master regulator of antioxidant defenses, NRF2. Drugs that induce NRF2 expression and activation can restore cell and mitochondrial function by increasing endogenous cell antioxidant defenses such as SOD1 and SOD2.

FIG. 31 shows non-limiting, exemplary graphs of data and a Western blot.

FIG. 32 shows a non-limiting schematic of the influence of lysosomal and mitochondrial dysfunction on development of cell senescence and the hallmark pathology of ocular diseases such as AMD.

FIG. 33 shows a schematic (doi.org/10.1111/febs.15570) representation of the pathological processes associated with aging cells that culminate in cell senescence and disease development. Areas highlighted with asterisks indicate pathological processes observed in cells that have decreased 5-HT2A receptor function, including morphological changes, metabolic dysfunction, cell cycle abnormalities, and lysosomal dysfunction.

FIG. 34 shows non-limiting, exemplary images of ARPE-19 before and after ROS induction.

FIG. 35 shows non-limiting, exemplary graphs of 5-HT2AR knockouts and 5-HT2AR pharmacological agonism/antagonism.

FIG. 36 shows representative images of effects of hyperglycemia on RPE cells. ARPE-19 cells cultured under either physiological or High Glucose/Nutrient Rich Conditions were assessed for the presence of senescent cells by staining cells for the presence of SA-β-Gal. Hyperglycemic conditions greatly enhanced the number cells exhibiting a cell senescent phenotype.

FIG. 37 shows non-limiting, representative data. Consistent with HyperGlycemic Induction of Cellular Senescence and Aging-Associated Diseases in the Diabetic Population, ARPE-19 Cells in Nutrient Rich Media have Higher Proportions of Senescent Cells Compared to ARPE-19 Cells in Physiologically Relevant Media. ARPE-19 cells in physiologically relevant media (HPLM; 7% FCS), or nutrient rich media (DMEM; 7% FCS), were seeded overnight prior to being replaced with starvation media (HPLM or DMEM 0.5% FCS). After overnight incubation in starvation media, cells were swapped to either HPLM or DMEM 7% FCS media (HPLM→HPLM; HPLM→DMEM; DMEM→DMEM for 72-hours. After the 72-hour incubation, cells were fixed and stained for SA-β-Gal for 6-hours. Cells nuclei were then stained with DAPI for total-cell enumeration. SA-β-Gal positive cells, and total cells per frame, were quantified by microscopy.

FIG. 38 shows non-limiting exemplary graphs of glucose concentration for intracellular ROS and senescence-associated B-glactosidase.

FIG. 39 shows a non-limiting, exemplary schematic of aspects described herein. Illustration adapted in part from doi.org/10.1111/febs.15570.

FIG. 40 is intentionally left blank.

FIG. 41 shows a ribbon diagram of 5-HT2A.

FIG. 42 is intentionally left blank.

FIG. 43 shows imagines of AcGFP1+ cells and a magnified dsRed2+ cell.

FIG. 44 shows non-limiting, representative schematic of the experimental approach taken to visualize mitochondria in live cells in FIG. 45 and FIG. 46.

FIG. 45 shows non-limiting exemplary data. Representative 5-HT2A Receptor Agonist, R-DOI, Increases Mitochondrial Biogenesis and Quality by Inducing Mitochondrial Fusion at low nM concentrations thereby reversing adverse processes that underlie development and progression of the Pathophysiology of Age-Associated Ocular and Neurodegenerative Diseases. ARPE cells transfected with plasmids that express either a red or green fluorescent protein biomarker that marks mitochondria were plated and treated with R-DOI @100 nM, 50 nM or 10 nM. 48 hours post treatment, mitochondrial morphology was evaluated by fluorescent microscopy. R-DOI treatment at all concentrations exhibited increased mitochondrial biomass with a non-fragmented fused morphology indicative of a higher quality and functioning mitochondria. Assessments indicated that extremely low levels of 5-HT2A agonists, increased both mitochondrial biomass and potential, increasing mitochondrial health, which is normally disrupted with aging-associated disease processes.

FIG. 46 shows non-limiting, representative data. Representative 5-HT2A Receptor Agonist, R-DOI, Increases Mitochondrial Biogenesis and Quality by Inducing Mitochondrial Fusion at low nM concentrations thereby reversing adverse processes that underlie development and progression of the Pathophysiology of Age-Associated Ocular and Neurodegenerative Diseases. Consistent with the Maintenance of Functional Mitochondria and Mitochondrial Health, Serotonin 2A Receptor Agonism, Like with R-DOI, Increases Enzymatic BioMarkers of Mitochondrial Fusion (Mitofusin2) and Biogenesis (TOMM20). ARPE-19 cells were grown in DMEM 7% FCS containing the indicated concentrations of R-DOI. 48 h post treatment, cell samples were lysed and normalized prior to running western blots to evaluate mitofusin2 and TOMM20 protein expression. R-DOI treatment at all concentrations exhibited increased mitochondrial biomass (TOMM20) with a non-fragmented fused morphology indicative of a higher quality and functioning mitochondria that were marked on western blots by the increased expression of Mitofusin 2, an enzymatic biomarker that increases fused high quality mitochondria. Assessments indicated that extremely low levels of 5-HT2A agonists, increased both mitochondrial biomass and mitochondrial fusion, increasing mitochondrial health, which is normally disrupted with aging-associated disease processes. Mitofusion2 functions to regulate mitochondrial fusion. TOMM20 imports proteins through the outer membrane and is a marker of biogenesis and number.

FIG. 47 shows a chemical reaction for an assay used herein.

FIG. 48 shows an exemplary graph of % ATP-Associated Luciferase Activity Relative to Mock Tx.

FIG. 49 shows the structure of 5-hydroxytryptamine and a ribbon diagram of the 5-HT2A receptor.

FIG. 50 shows non-limiting, exemplary schematic (adapted from genecopoeia.com/product/crispr-cas9/) of how the 5-HT2A receptor knock down and knock out cells were engineered via CRISPR/CAS9 and screened for loss of 5-HT2A receptor expression and purity of cell lines.

FIG. 51 shows exemplary data of 5-HT2A receptor expression.

FIG. 52 is intentionally left blank.

FIG. 53 shows non-limiting, exemplary data. Representative data of various ARPE-19 cells generated that are knockdown or knocked out for 5-HT2A receptor expression, with the corresponding % of expression compared to wildtype control for each.

FIG. 54 shows a schematic of techniques described herein.

FIG. 55 shows exemplary data. Specific quantitative detection of 5-HT2A receptor expression in ARPE-19 retinal pigmented epithelial cells was optimized by flow cytometric analysis with optimal primary antibody concentrations determined to be 0.5 ug/ml to 1 ug/ml.

FIG. 56 shows exemplary data. Specific quantitative detection of 5-HT2A receptor expression in ARPE-19 retinal pigmented epithelial cells was optimized by flow cytometric analysis with optimal primary antibody concentrations determined to be 0.5 ug/ml to 1 ug/ml.

FIG. 57 shows non-limiting, exemplary schematic (doi:10.1007/s00018-021-03796-9) of the pathophysiological processes involved in the development of AMD.

FIG. 58 shows the structure of 5-hydroxytryptamine.

FIG. 59 shows exemplary images of the effects of decreased 5-HT2A receptor function on cell morphology. Consistent with Irregular Morphology and the Disrupted RPE in AMD, Diabetic Retinopathy, and Other Retinopathies, Serotonin 2A Receptor Knockouts have Increased Cell Surface Area Compared to the ARPE-19 Wildtype. ARPE-19 cells and ARPE-19 cells specifying 5-HT2AR knockout/knockdowns were plated in nutrient rich media (DMEM; 7% FCS) overnight. Cells were stained with a cell surface dye and nuclei were stained with DAPI. Images were acquired via fluorescent microscopy. Cell surface area was determined by analyzing image regions denoted by the fluorescently stained green cell areas. Cell surface area was determined and calculated through ImageJ pixel content analysis. Decreases in 5-HT2A receptor expression and function are associated with hallmark pathologies associated with age-related disease development, including increases in cell size/morphology that disrupts barrier integrity and cellular functions.

FIG. 60 shows images of ARPE-19. A Hallmark of Age-Associated Ocular and Neurodegenerative Disorders is Cytoskeletal Alteration and Induction of Stress Fiber Formation. Consistent with a Damaged RPE Monolayer from Retinal Contraction and Cytoskeletal Alterations Observed in AMD, Diabetic Retinopathy, and Other Retinopathies, Serotonin 2A Receptor Knockouts Exhibit Cytoskeletal Dysfunction and the Induction of Cytoskeletal Stress Fibers. ARPE-19 cells and ARPE-19 cells specifying 5-HT2AR knockouts were plated in nutrient rich media (DMEM: 7% FCS) overnight. Cells were fixed, permeabilized, and stained phalloidin to visualize actin fiber cytoskeletal structures via fluorescent microscopy. Normal healthy RPE, are small compact structures with an absence of stress fibers across the cytoplasm. The majority of actin rims the cell surface. However, in ocular and neurodegenerative diseases the formation of stress fibers are a hallmark and biomarker of disease development and progression. Age-related decreases in 5-HT2A receptor expression correlate with onset of disease development. Similarly, RPE cells knockdowned/knockout for 5-HT2A receptor expression and function exhibit high levels of stress fiber formation that would be associated with disease development.

FIG. 61 shows exemplary experimental images. A Hallmark of Age-Associated Ocular and Neurodegenerative Disorders is Cvtoskeletal Alteration and Induction of Stress Fiber Formation. Consistent with a Damaged RPE Monolayer from Retinal Contraction and Cytoskeletal Alterations Observed in AMD, Diabetic Retinopathy, and Other Retinopathies, Serotonin 2A Receptor Knockouts Exhibit Cytoskeletal Dysfunction and the Induction of Cytoskeletal Stress Fibers. Displayed is a magnified view of cells similar to FIG. 60. ARPE-19 cells and ARPE-19 cells specifying 5-HT2AR knockouts were plated in nutrient rich media (DMEM; 7% FCS) overnight. Cells were fixed, permeabilized, and stained phalloidin to visualize actin fiber cytoskeletal structures via fluorescent microscopy. Normal healthy RPE, are small compact structures with an absence of stress fibers across the cytoplasm. The majority of actin rims the cell surface. However, in ocular and neurodegenerative diseases the formation of stress fibers are a hallmark and biomarker of disease development and progression. Age-related decreases in 5-HT2A receptor expression correlate with onset of disease development. Similarly, RPE cells knockdowned/knockout for 5-HT2A receptor expression and function exhibit high levels of stress fiber formation that would be associated with disease development.

FIG. 62 shows exemplary illustrations of f-actin stress fibers (doi.org/10.1002/med.21270) and a retinal detachment (mayoclinic.org/diseases-conditions/retinal-detachment/symptoms-causes/syc-20351344).

FIG. 63 shows exemplary experimental data. A Hallmark of Age-Associated Ocular and Neurodegenerative Disorders is Cvtoskeletal Alteration and Induction of CONTRACTILE Stress Fiber Formation. Expression and activation via phosphorylation of Myosin Light Chain (MLC), is a biomarker of disease development. Serotonin 2A Receptor Knockouts Exhibit Cytoskeletal Dysfunction and the Induction of MLC phosphorylation, indicative of disease development. ARPE-19 cells and ARPE-19 cells specifying 5-HT2AR knockouts were plated in nutrient rich media (DMEM; 7% FCS) overnight. Cells were fixed, permeabilized, and stained phalloidin and α-phosphorylated-Myosin light chain. Cytoskeletal elements were then visualized by fluorescent microscopy to visualize actin fiber cytoskeletal structures (phallodin, red) and contractile phosphorylated myosin light chain (green) via fluorescent microscopy. Normal healthy RPE, are small compact structures with an absence of stress fibers across the cytoplasm. The majority of actin and phosphorylated myosin light chain rims the cell surface. However, in ocular and neurodegenerative diseases the formation of stress fibers and the association of activated phosphorylated MLC on these stress fibers are a hallmark and biomarker of disease development and progression. Age-related decreases in 5-HT2A receptor expression correlate with onset of disease development. Similarly, RPE cells knockdowned/knockout for 5-HT2A receptor expression and function exhibit high levels of stress fiber formation (red), as well as association of phosphorylated MLC (green) on stress fibers that would be associated with disease development.

FIG. 64 has been left intentionally blank.

FIG. 65 shows non-limiting, exemplary data. Decreased 5-HT2A receptor Expression and Activity Results in Buildup of Autofluorescent Disease-inducing Drusen Characteristic of Aging-Associated Disease Processes. Autofluorescent Lipofuscin/Drusen Accumulation is a biomarker of disease development and progression in Neurodegenerative and Ocular diseases, such as AMD, Diabetic Retinopathy, and Other Retinopathies. Consistent with Lysosomal Dysfunction and Inadequate Waste Degradation in AMD, Diabetic Retinopathy, and Other Retinopathies, Serotonin 2A Receptor Knockouts Have Increased Autofluorescent properties compared to the ARPE-19 Wildtype. Dysregulation of the autophagy-lysosomal system is well-described in neurodegenerative disorders, including AMD. The inability to efficiently degrade protein/lipids results in the buildup of auto-fluorescent deposits in the RPE, termed drusen bodies. Increased autofluorescence within cells indicates and inability to clear deleterious toxic proteins and lipids from the environment. ARPE-19 cells and ARPE-19 cells specifying 5-HT2AR knockout were plated in nutrient rich media (DMEM; 7% FCS) overnight. Cells were analyzed via flow cytometry for autofluorescent characteristics in the FL1 channel by determining mean fluorescent intensity per cell. 5-HT2A knockouts exhibited increased autofluorescence in congruence with the pathophysiological processes associated with ocular and neurogenerative diseases of aging.

FIG. 66 shows a non-limiting schematic representation of how diabetic hyperglycemic conditions can induce increased cellular disease processes like induction of reactive oxygen species and cell senescence. As a consequence, in ocular and neuronal tissues, pathophysiological processes that result in retinopathies, AMD, and neurodegenerative diseases result.

FIG. 67 shows a schematic representation of how the presence of reactive oxygen species are quantitatively detected within cells via the Promega ROS-Glo detection solution (Promega.com/products/cell-health-assays/oxidative-stress-assays/ros_glo-h2o2-assay/?catNum=G8821 #overview).

FIG. 68 shows a non-limiting, representative graph. Hyperglycemic conditions induce increased disease promoting ROS production that is abrogated by low nM concentrations of the Representative 5-HT2A Receptor Agonist. R-DOI Returning to Base-line Homeostatic Levels. ARPE-19 cells were plated using physiological media (HPLM; 7% FCS) overnight prior to starvation (HPLM 0.5% FCS) overnight with or without 10 nM R-DOI. Cells were then replaced with HPLM 7% FCS containing a range of glucose concentrations spanning diabetic blood glucose levels (5 mM, 10 mM, 20 mM, and 30 mM) with or without the addition of 10 nM R-DOI for 72-hours. Cells were then lysed to quantify intracellular H₂O₂ levels via bioluminescence as described above. Low dose 5-HT2A agonists abrogate the hyperglycemic induced induction od ROS that is pathophysiological for development and progression of neurodegenerative and ocular age-related diseases, especially within diabetic patients, or patients that have conditions that induce increased levels of blood glucose. Pathological glucose concentrations increase intracellular H₂O₂. 5-HT2AR agonism alleviates glucose-induced intracellular H₂O₂ at diabetic glucose concentrations.

FIG. 69 shows a non-limiting, representative graph. Decreased 5-HT2A receptor Expression and Activity Results in Increased Levels of Intracellular Reactive Oxygen Species. The phenotype is consistent with the Aging-associated Neurodegeneration and Ocular Diseases. ARPE-19 cells and ARPE-19 cells specifying a 5-HT2AR knockout were plated in nutrient rich media (DMEM; 7% FCS) and grown overnight. Cells were then lysed to quantify intracellular H₂O₂ levels via bioluminescence using a promega reactive oxygen species quantification kit according to manufacturer's instructions. Knockdown of 5-HT2A receptor expression results in increased presence of reactive oxygen species that can damage cellular proteins, lipids, etc. and promote development and progression of age-related disease processes. Increased ROS can cause damage to DNA, protein, and lipids which are characteristic of age-related diseases, including AMD.

FIG. 70 shows non-limiting, representative data. Decreased 5-HT2A receptor Expression and Activity Results in Disease-inducing Decreases in Levels of Antioxidant Enzymes, SOD1 and SOD2, Indicating Poor Processing of Intracellular Reactive Oxygen Species. The phenotype is consistent with the Aging-associated Neurodegeneration and Ocular Diseases. ARPE-19 cells and ARPE-19 cells specifying a 5-HT2AR knockout were plated in nutrient rich media (DMEM; 7% FCS) and grown to confluency prior to collection of cell lysates. Protein levels were normalized among samples and run on a western blot for comparison of the biomarkers of cellular antioxidant expression superoxide dismutase 1 and 2 enzymes or a cellular protein loading control GAPDH. Band intensity was quantified via ImageJ, normalized to relative GAPDH expression and compared to WT ARPE cells that express normal levels of 5-HT2A receptor. Knockdown of 5-HT2A receptor expression results in decreased expression of endogenous anti-oxidant proteins which would be indicative of a decreased ability to cope with disease promoting oxidative stress.

FIG. 71 shows a non-limiting summary of data associated with the disease processes induced by decreased 5-HT2A receptor function in retinal pigmented epithelial (RPE) cells.

FIG. 72 shows non-limiting, exemplary data. Serotonin 2A Receptor Repression, using the 5-HT2A antagonist, M100,907, Increases Proportions of Cellular Senescence. Conversely, low nM dose Serotonin 2A Receptor Agonism, with R-DOI, Decreases Basal Levels of Cellular Senescence. ARPE-19 cells in nutrient rich media (DMEM; 7% FCS) were seeded overnight prior to being replaced with starvation media (DMEM; 0.5% FCS) containing 10 nM R-DOI or 10 nM M100,907. After overnight incubation in starvation media with drug, cells were swapped to DMEM 7% FCS media for 72-hours. After the 72-hour incubation, cells were fixed and stained for SA-β-Gal for 6-hours. Cells nuclei were then stained with DAPI for total-cell enumeration. SA-β-Gal positive cells, and total cells per frame, were quantified by microscopy.

FIG. 73 shows images of ARPE-19 cells.

FIG. 74 shows a graph of % B-gal+cells per field.

FIG. 75 shows a schematic of 5-HT2AR knockdown or pharmacological antagonism. Illustration adapted in part from doi.org/10.1111/febs.15570.

FIG. 76 shows an image of 5-HT2A receptors (doi: 10.1089/jp.2017.0125) and non-limiting, exemplary data of 5HT2A receptor expression

FIG. 77 shows representative data. The pathophysiological impact of the Age-Associated Decline in Serotonin 2A Receptor Expression was assessed by Generating CRISPR/Cas9 Site-Directed Knockout of the Serotonin 2A Receptor in RPE cells. A CRISPR/Cas9 system was utilized for site-directed knockout of the serotonin 2A receptor in ARPE-19 cells. Purification of cell-lines specifying either single or double allele knockouts of 5-HT2AR were achieved through FACS sorting. Assessment of levels of 5-HT2A Receptor expression was determined by western blot analysis. Cell lysates from each isolated cell line were collected and normalized to protein content prior to running western blot analysis specific for either serotonin 2A receptor or cellular GAPDH protein expression. Levels of Serotonin 2A receptor expression was quantified by densitometry of western bands on collected images, normalized to GAPDH. Values of expression relative to ARPE-19 wildtype set at 100% were determined for each of the serotonin 2A receptor knockout cell-lines generated.

FIG. 78 shows exemplary images of the effects of decreased 5-HT2A receptor function on cell morphology. Consistent with Irregular Morphology and the Disrupted RPE in AMD, Diabetic Retinopathy, and Other Retinopathies, Serotonin 2A Receptor Knockouts have Increased Cell Surface Area Compared to the ARPE-19 Wildtype. ARPE-19 cells and ARPE-19 cells specifying 5-HT2AR knockout/knockdowns were plated in nutrient rich media (DMEM; 7% FCS) overnight. Cells were stained with a cell surface dye and nuclei were stained with DAPI. Images were acquired via fluorescent microscopy. Cell area was determined by analyzing image regions denoted by the fluorescently stained green cell areas. Cell surface area was determined and calculated through ImageJ pixel content analysis. Decreases in 5-HT2A receptor expression and function are associated with hallmark pathologies associated with age-related disease development, including increases in cell size/morphology that disrupts barrier integrity and cellular functions.

FIG. 79 shows representative data on lysosomal dysfunction and lipofuscin accumulation in 5-HT2A receptor defective cells. Consistent with Lysosomal Dysfunction and Inadequate Waste Degradation in AMD, Diabetic Retinopathy, and Other Retinopathies, Serotonin 2A Receptor Knockouts Have Increased Lysosomal Biomass and Lipofuscin/Drusen accumulation Compared to the ARPE-19 Wildtype. (LEFT GRAPH) Cells were stained for lysosomal content using lysotracker dyes, suspended, and analyzed via flow cytometry for relative lysomal content by determining mean fluorescent intensity per cell. 5-HT2A knockouts exhibited increased lysosomal content in congruence with the pathophysiological processes associated with ocular and neurogenerative diseases of aging. (RIGHT GRAPH) The inability to efficiently degrade protein/lipids results in the buildup of auto-fluorescent deposits in the RPE, termed drusen bodies. Increased lysosomal content can indicate an abundance and failure to clear deleterious toxic proteins and lipids from the environment. Cells were analyzed via flow cytometry for autofluorescent characteristics in the FL1 channel by determining mean fluorescent intensity per cell. 5-HT2A knockouts exhibited increased autofluorescence in congruence with the pathophysiological processes associated with ocular and neurogenerative diseases of aging.

FIG. 80 shows non-limiting, exemplary graphs of expression data, intracellular ROS, and a representative Western blot.

FIG. 81 shows a representative schematic. Decreased 5-HT2a Receptor Expression and Activity Results in Pathophysiological aging of cells And a Senescent Phenotype: Activation of 5-HT2A Receptor Function inhibits Hyperglycemia Induced Cell Senescence Associated with Development and Progression of Neurodegenerative and Ocular Diseases. Senescence is the cellular equivalent to aging. Senescent cell characteristics include limited proliferation, enlarged cell size, excessive ROS, metabolic and proteolytic dysfunction, senescence-associated secretory phenotype (pro-inflammatory cytokines), nutrient imbalances, telomere attrition, DNA damage, and epigenetic dysregulation. In addition to these characteristics, senescence-associated β-galactosidase (SA-O-Gal) staining is the gold standard for quantifying cellular senescence. Senescence is the cellular equivalent to aging. Senescence can refer to ‘irreversible’ cell-cycle arrest. Senescent cell characteristics include limited proliferation, enlarged cell size, excessive ROS, metabolic and proteolytic dysfunction, senescence-associated secretory phenotype (pro-inflammatory cytokines), nutrient imbalances, telomere attrition. DNA damage, and epigenetic dysregulation. In addition to these characteristics, senescence-associated β-galactosidase(SA-β-Gal) staining is the gold standard for quantifying cellular senescence.

FIG. 82 shows an exemplary graph of expression and age. 5-HT2A Receptor Expression Levels within the Retina STRONGLY & Negatively Correlates with Age: Linear Filtered Counts. Serotonin 2A Receptor Expression Is Significantly Lower in Patient Populations Prone to Developing Age-Related Retinopathies and Neurodegenerative Diseases. Retrospective analysis was performed from RNAseq data from clinically-derived ocular retinal samples from healthy individuals ranging from 30 to 95 years old. Serotonin 2 receptor expression was evaluated in context to age using linear filtered counts. Given our findings that 5-HT2A receptor function is critical to prevent development of Age-Related Diseases, the decline in receptor with age dictates that a target patient population prior to 60 can be amenable to treatment, and post 60 years of age, 5-HT2A agonist drug levels can need to be increased to adequately stimulate the decreasing levels of 5-HT2A receptors.

FIG. 83 shows an exemplary graph of expression and age. 5-HT2A Receptor Expression Levels within the Retina STRONGLY & Negatively Correlates with Age: Log 2 Filtered Counts. Serotonin 2A Receptor Expression Is Significantly Lower in Patient Populations Prone to Developing Age-Related Retinopathies and Neurodegenerative Diseases. Retrospective analysis was performed from RNAseq data from clinically-derived ocular retinal samples from healthy individuals ranging from 30 to 95 years old. Serotonin 2 receptor expression was evaluated in context to age using log 2 filtered counts. Given our findings that 5-HT2A receptor function is critical to prevent development of Age-Related Diseases, the decline in receptor with age dictates that a target patient population prior to 60 can be amenable to treatment, and post 60 years of age, 5-HT2A agonist drug levels can need to be increased to adequately stimulate the decreasing levels of 5-HT2A receptors.

FIG. 84 shows an exemplary graph of expression and age. 5HT2A Receptor Expression is Significantly Decreased within the Retina after 60 Years of Age. Serotonin 2A Receptor Expression Is Significantly Lower in Patient Populations Prone to Developing Age-Related Retinopathies and Neurodegenerative Diseases. Retrospective analysis was performed from RNAseq data from clinically-derived ocular retinal samples from healthy individuals ranging from 30 to 95 years old. Serotonin 2 receptor expression was evaluated in context to age grouping expression levels in retinas of patients less than 51 years of age or over 60 years of age. This delineation in age groups was selected as the critical target population that present with age-related ocular or neurodegenerative diseases begin pathophysiological presentation at this transition age. Given our findings that 5-HT2A receptor function is critical to prevent development of Age-Related Diseases, the decline in receptor with age dictates that a target patient population prior to 60 would be amenable to treatment, and post 60 years of age, 5-HT2A agonist drug levels can need to be increased to adequately stimulate the decreasing levels of 5-HT2A receptors.

FIG. 85 shows an exemplary graph of expression and age. Expression of 5HT2C Receptor Expression Levels within the Retina Negatively Correlates with Age; However, Changes are not as Significant or as Strong as with 5HT2A Receptor. Retrospective analysis was performed from RNAseq data from clinically-derived ocular retinal samples from healthy individuals ranging from 30 to 95 years old. Serotonin 2 receptor expression was evaluated in context to age using log 2 filtered counts.

FIG. 86 shows exemplary graphs. As Expression of 5HT2A Receptor Expression Levels Decreases within the Retina Expression of Genes that Influence Retinal Metabolism, Diabetic Disease Processes & Inflammation Increase with Age. Retrospective analysis was performed from RNAseq data from clinically-derived ocular retinal samples from healthy individuals ranging from 30 to 95 years old. Expression of representative biomarkers of retinal metabolism and inflammation was evaluated in context to age using log 2 filtered counts.

FIG. 87 shows exemplary graphs. 5-HT2A Receptor Function is Required for Healthy Replication of Retinal Pigmented Epithelia: Serotonin Enhances Cellular Health and Replication through the 5-HT2A Receptor. ARPE-19 cells were plated in nutrient rich media, DMEM, containing 7% dialyzed FCS and then starved in DMEM media containing 0.5% dialyzed FCS. Cells were then changed to 7% dialyzed FCS DMEM media containing physiological relevant levels of serotonin at varying concentrations and grown for 72-hours. Cell numbers were determined through DNA staining with DAPI and fold change in growth was determined from baseline readings at 24-hours. To assess if serotonin enhances cell health and proliferation specifically through the 5-HT2A receptor, the 5-HT2A receptor antagonist, M100,907 at 10 nM was included during serotonin induction. Inhibition of 5-HT2A receptor function using M100,907 antagonist abrogated serotonin-mediated enhanced replication, indicating that serotonin's positive role on cellular function occurs through the 5-HT2A receptor. This inhibition mimics the effects observed in patient samples that exhibit a decline in 5-HT2A receptor expression and function observed in aging individuals.

FIG. 88 shows an exemplary growth curve. 5-HT2A Receptor Function is Required for Healthy Replication of Retinal Pigmented Epithelia: Serotonin Enhances Cellular Health and Replication through the 5-HT2A Receptor. ARPE-19 cells were plated in nutrient rich media, DMEM, containing 7% dialyzed FCS and then starved in DMEM media containing 0.5% dialyzed FCS. Cells were then changed to 7% dialyzed FCS DMEM media containing physiological relevant levels of serotonin at varying concentrations and grown for 72-hours. Cell numbers were determined through DNA staining with DAPI and fold change in growth was determined from baseline readings at 24-hours. To assess if serotonin enhances cell health and proliferation specifically through the 5-HT2A receptor, the 5-HT2A receptor antagonist, M100,907 at 10 nM was included during serotonin induction. Inhibition of 5-HT2A receptor function using M100,907 antagonist abrogated serotonin-mediated enhanced replication, indicating that serotonin's positive role on cellular function occurs through the 5-HT2A receptor. This inhibition mimics the effects observed in patient samples that exhibit a decline in 5-HT2A receptor expression and function observed in aging individuals.

FIG. 89 shows non-limiting, representative schematic. Data described herein can assess the role of 5-HT2A receptor presence and function on mitochondrial dynamics, mitochondrial function, energy production, and alleviation of mitochondrial dysfunction-associated diseases.

FIG. 90 shows non-limiting, representative data. Representative RT-PCR data detailing that low dose representative 5-HT2A receptor agonist R-DOI increases protective NAD levels by upregulating biosynthetic pathways associated with NAD synthesis and salvage. Conversely, higher doses of the representative 5-HT2A agonist R-DOI causes downregulation of these pathways and decreased NAD levels.

FIG. 91 shows non-limiting exemplary data and explanatory schematic. Sirtuins utilize NAD to suppress pathophysiological processes of aging, including diabetic retinopathies, AMD and Neurodegeneration. Representative western analysis of Sirtuin (SIRT) 1 and 3 expression following cellular treatment with the representative 5-HT2A Receptor agonist R-DOI at varying concentrations. Protective SIRT1 and 3 expression was induced by 5-HT2A agonism and counterintuitively increased as concentrations decreased to nM concentrations.

FIG. 92 shows an exemplary illustration of the 5-HT2A receptor effects on mitochondria (Fanibunda et al., 116 (22) 11028-11037 doi.org/10.1073/pnas.1821332116).

FIG. 93 shows non-limiting exemplary data. Treatment of cells with representative 5-HT2A agonists, R-DOI or 2C-iBu (ELE02) at low nM concentrations increases protection of cells from aging and oxidative stress associated diseases, that the activation of AMP kinase, which results in increased protective responses, including NAD production, SIRT expression, and NRF2 expression.

FIG. 94 shows non-limiting, exemplary data. Representative 5-HT2A Receptor Agonist, R-DOI, Increases Mitochondrial Biogenesis and Function at low nM to sub nanomolar concentrations thereby reversing adverse processes that underlie development and progression of the Pathophysiology of Age-Associated Ocular and Neurodegenerative Diseases. ARPE cells were plated and treated with 10 fold dilutions of R-DOI from 100 nM to 0.001 nM. 48 hours post treatment, mitochondrial biomass was assessed either by staining for mitochondria using mitotracker (green, left panel) or via flow cytometry using MitoTracker Red CMOXROS that assesses both mitochondrial biomass and mitochondrial potential. Assessments indicated that extremely low levels of 5-HT2A agonists, increased both mitochondrial biomass and potential, increasing mitochondrial health, which is normally disrupted with aging-associated disease processes.

FIG. 95 shows non-limiting, exemplary data and schematics. Representative 5-HT2A Receptor Agonist, R-DOI Increases Mitochondrial Function And ATP Production at low nM and sub nanomolar concentrations thereby reversing adverse processes that underlie development and progression of the Pathophysiology of Age-Associated Ocular and Neurodegenerative Diseases. ARPE cells were plated and treated with 10 fold dilutions of R-DOI from 100 nM to 0.001 nM. 48 hours post treatment, mitochondrial function and ATP production was assessed by Seashorse. Assessments indicated that extremely low levels of 5-HT2A agonists, increased ATP production and mitochondrial respiration.

FIG. 96 shows non-limiting, exemplary data. Representative 5-HT2A Receptor Agonists, R-DOI and 2C-iBu Increase Mitochondrial Function And ATP Production at low nM concentrations thereby reversing adverse processes that underlie development and progression of the Pathophysiology of Age-Associated Ocular and Neurodegenerative Diseases. ARPE cells were plated and treated with 10 fold dilutions of either R-DOI or 2C-iBu from 100 nM to 0.1 nM. The levels of ATP were determined within treated cells relative to Mock treated ARPE-19 cells. ATP levels were determined using a promega quantitative ATP luciferase assay according to manufacturers instructions.

FIG. 97 shows non-limiting, exemplary data, non-limiting, exemplary data. Consistent with the Pathophysiology of Age-Associated Ocular and Neurodegenerative Diseases, 5-HT2A Receptor KNOCKDOWNS DECREASE Mitochondrial Function And ATP Production. ARPE cells or ARPE cells specifying knockdown/knockouts of 5-HT2A receptor were plated. The levels of ATP were determined within treated cells relative to Mock treated ARPE-19 cells. ATP levels were determined using a promega quantitative ATP luciferase assay according to manufacturers instructions.

FIG. 98 shows a non-limiting, representative schematic and Western Blot. Representative 5-HT2A Receptor Agonist, R-DOI, Increases Expression of the master regulator of antioxidant gene expression NRF-2 at low nM concentrations thereby initiating cellular processes that would reversing adverse oxidative stress processes that underlie development and progression of the Pathophysiology of Age-Associated Ocular and Neurodegenerative Diseases. ARPE cells were plated and treated with 10-fold dilutions of R-DOI from 1000 nM to 1 nM. Following an overnight treatment, cells were incubated with hydrogen peroxide (H₂O₂) to mimic an oxidative stress induced disease event. 24 hours later, cell lysates were collected and protein levels were normalized among samples and run on a western blot for comparison of NRF-2 expression relative to a cellular protein loading control, b-actin. While doses increased protective NRF-2 expression, lower doses increased NRF-2 to the highest levels, accentuating the need for appropriate dose ranges in facilitating treatment effects.

FIG. 99 shows non-limiting, representative data. Representative 5-HT2A Receptor Agonists, R-DOI and 2C-iBu Inhibit Blue Light and Oxidative Stress Induced Mitochondrial Fragmentation/Damage at low nM concentrations thereby reversing adverse processes that underlie development and progression of the Pathophysiology of Age-Associated Ocular and Neurodegenerative Diseases. ARPE cells grown under hyperglycemic conditions were plated and mock treated or treated with R-DOI or 2C-iBu at 100 nM. 48 hours post treatment, mitochondria in live cells were visualized by staining mitochondria with mitotracker green (green). Once stained, live ARPE cells were exposed to focused blue light at 400 nm and the ability of drugs to protect against blue light damage-induced mitochondrial fragmentation was assessed. Assessments indicated that treatment of RPE with nM levels of 5-HT2A agonists, protected mitochondria against blue light damage and maintained mitochondrial health, which is normally disrupted with aging-associated disease processes.

FIG. 100 shows non-limiting, representative data and schematics. Representative 5-HT2A Receptor Agonist, R-DOI Inhibits Blue Light Induced Mitochondrial-Mediated Oxidative Stress at low sub-nanomolar concentrations thereby reversing adverse processes that are associated with retinal exposure to blue light. ARPE cells grown under hyperglycemic conditions were plated and mock treated or treated with R-DOI at 0.001 nM. 24 hours post treatment, live ARPE cells were labeled with the mitochondrial oxidative stress indicator dye Mitosox that becomes fluorescent red under mitochondria-induced oxidative stress conditions. Once stained, live ARPE cells were exposed to focused blue light at 400 nm for 12 seconds and the ability of drugs to protect against blue light damage-induced mitochondrial oxidative stress induction was assessed. In the absence of 5HT2A agonists, there was a clear increase in red fluorescence upon blue light exposure. However, R-DOI at 0.001 nM concentrations abrogated mitochondrial oxidative stress responses a pathophysiological process associated with development and progression of age-related diseases. Assessments indicated that treatment of RPE with low sub nanomolar levels of 5-HT2A agonists, protected cells and mitochondria against blue light damage, induction of mitochondrial oxidative stress, and maintained mitochondrial health.

FIG. 101 shows non-limiting, representative data and schematics. Representative 5-HT2A Receptor Agonist, R-DOI, Decreases Oxidative stress induced lipid peroxidation at low nM concentrations thereby reversing adverse processes that underlie development and progression of the Pathophysiology of Age-Associated Ocular and Neurodegenerative Diseases. ARPE cells were plated and mock treated or treated with the 5HT2A agonist R-DOI at 100 nM or 1 nM concentrations. Following an overnight incubation, live ARPE cells were labeled with an lipid peroxidation indicator dye BODIPY that changes fluorescence from red to green as it becomes oxidized. Cells were subsequently treated with 100 uM cumene hydroperoxide to induce lipid peroxidation and cellular oxidative stress and imaged in the red and green channels to assess the protective effects of 5HT2A agonists on lipid peroxidation. In the absence of 5HT2A agonists, there was a clear increase in green fluorescence present following treatment with cumene hydroperoxide. However, R-DOI at 100 nM and 1 nM concentrations abrogated lipid peroxidation a pathophysiological process associated with development and progression of age-related diseases.

FIG. 102 shows non-limiting, representative images of cell viability following oxidative stress. A549 cells were treated with the indicated concentrations of R-DOI (panel A) or 2C-iBu (panel B) and subsequently exposed to cumene peroxide to oxidatively stress cells. Cell viability was visualized by staining with Hoescht (Blue) to mark total cells (live plus dead) and propidium iodide (red) to mark dead cells. Treatment with 5-HT2A agonists protected cells from oxidative stress-induced cell death.

FIG. 103 shows a representative graph of expression and age. Expression of Other Serotonin Receptors, including 5-HT2B, within the Retina DO NOT Correlate with Age: Log 2 Filtered Mean Counts. Retrospective analysis was performed from RNAseq data from clinically-derived ocular retinal samples from healthy individuals ranging from 30 to 95 years old. Serotonin 2 receptor expression was evaluated in context to age using log 2 filtered counts.

FIG. 104 This figure is intentionally left blank.

FIG. 105 shows an exemplary illustration. Described herein are data that assesses the effects of 5-HT2A receptor Expression and function on Metabolic dysfunction, Oxidative Stress And Aging-associated diseases. Generation of Reactive Oxygen Species is a driver of Development and Progression of Age-related diseases that can be enhance by a hyperglycemic patient condition.

FIG. 106 shows non-limiting, representative data. Decreased 5-HT2a Receptor Expression and Activity Results in Pathophysiological aging of CELLS And a Senescent Phenotype: Serotonin 2A Receptor Knockouts Have High Levels of Cellular Senescence Compared to the ARPE-19 Wildtype. ARPE-19 cells and ARPE-19 cells specifying 5-HT2AR knockout in nutrient rich media (DMEM; 7% FCS) were seeded overnight prior to being replaced with starvation media (DMEM; 0.5% FCS). After overnight incubation in starvation media, cells were swapped to DMEM 7% FCS media for 72-hours. After the 72-hour incubation, cells were fixed and stained for SA-Q-Gal for 6-hours. Cells nuclei were then stained with DAPI for total-cell enumeration. SA-β-Gal positive cells, and total cells per frame, were quantified by microscopy.

FIG. 107 shows a non-limiting, exemplary graph. Consistent with Retinal Deterioration by Glucose-Induced Cellular Senescence in the Diabetic Population, Hyperglycemic conditions induce increased disease promoting Cellular Senescence that is abrogated by low nM concentrations of the Representative 5-HT2A Receptor Agonist, R-DOI Returning to Base-line Homeostatic Levels. ARPE-19 cells in physiologically relevant media (HPLM; 7% FCS) were seeded overnight prior to being replaced with starvation media (HPLM 0.5% FCS) containing 10 nM R-DOI. After overnight incubation in starvation media, cells were swapped to media containing diabetic blood glucose levels (HPLM 7% FCS containing 5 mM, 10 mM, 20 mM, or 30 mM glucose) for 72-hours. After the 72-hour incubation, cells were fixed and stained for SA-β-Gal for 6-hours. Cells nuclei were then stained with DAPI for total-cell enumeration. SA-β-Gal positive cells, and total cells per frame, were quantified by microscopy. Pathological/diabetic glucose concentrations induce cellular senescence. 5-HT2A receptor agonism alleviates glucose-induced senescence at diabetic blood glucose levels. Pathological/diabetic glucose concentrations induce cellular senescence. 5-HT2A receptor agonism alleviates glucose-induced senescence at diabetic blood glucose levels.

FIG. 108 shows non-limiting, exemplary data for R-DOI. Effects of specific phenethylamine 5-HT2A receptor agonists on reduction of Hyperglycemia induced Disease Promoting Cellular Senescence: Inhibition of Cell Senescence and Disease Processes are Drug and Dose Dependent. ARPE-19 cells in physiologically relevant media (HPLM; 7% FCS 5 mM Glucose) were seeded overnight prior to being replaced with starvation media (HPLM; 0.5% FCS; 5 mM Glucose) containing 1 uM, 100 nM, 10 nM, 1 nM, 0.1 nM, or no drug concentrations. After overnight incubation in starvation/drug media, cells were grown in diabetic conditions (HPLM; 7% FCS; 30 mM Glucose) with 1 uM. 100 nM, 10 nM, 1 nM. 0.1 nM, or no drug concentrations for 72-hours. After the 72-hour incubation, cells were fixed and stained for SA-f-Gal for 6-hours. Cells nuclei were then stained with DAPI for total-cell enumeration. SA-3-Gal positive cells, and total cells per frame, were quantified by microscopy. For each 5-HT2A agonist assessed, the % of senescent cells is expressed relative to the hyperglycemic control. In addition, the Fold reduction in cell senescence relative to hyperglycemic controls are also shown. NS=Not significant; * is significant to relative p value of number of stars.

FIG. 109 shows non-limiting, exemplary data for R-DOTFM. R-DOTFM enhances rather than inhibits Aging-Associated Disease Processes and Cellular Senescence. Inhibition of Cell Senescence and Disease Processes are Drug and Dose Dependent. ARPE-19 cells in physiologically relevant media (HPLM: 7% FCS; 5 mM Glucose) were seeded overnight prior to being replaced with starvation media (HPLM; 0.5% FCS: 5 mM Glucose) containing 1 uM. 100 nM, 10 nM, 1 nM, 0.1 nM, or no drug concentrations. After overnight incubation in starvation/drug media, cells were grown in diabetic conditions (HPLM; 7% FCS; 30 mM Glucose) with 1 uM, 100 nM, 10 nM, 1 nM, 0.1 nM, or no drug concentrations for 72-hours. After the 72-hour incubation, cells were fixed and stained for SA-R-Gal for 6-hours. Cells nuclei were then stained with DAPI for total-cell enumeration. SA-β-Gal positive cells, and total cells per frame, were quantified by microscopy. For each 5-HT2A agonist assessed, the % of senescent cells is expressed relative to the hyperglycemic control. In addition, the Fold reduction in cell senescence relative to hyperglycemic controls are also shown. NS=Not significant; * is significant to relative p value of number of stars.

FIG. 110 shows non-limiting, exemplary data for 2C-iBu. Effects of specific phenethylamine 5-HT2A receptor agonists on reduction of Hyperglycemia induced Disease Promoting Cellular Senescence: Inhibition of Cell Senescence and Disease Processes are Drug and Dose Dependent. ARPE-19 cells in physiologically relevant media (HPLM; 7% FCS; 5 mM Glucose) were seeded overnight prior to being replaced with starvation media (HPLM; 0.5% FCS; 5 mM Glucose) containing 1 uM, 100 nM, 10 nM, 1 nM, 0.1 nM, or no drug concentrations. After overnight incubation in starvation/drug media, cells were grown in diabetic conditions (HPLM; 7% FCS; 30 mM Glucose) with 1 uM, 100 nM, 10 nM, 1 nM, 0.1 nM, or no drug concentrations for 72-hours. After the 72-hour incubation, cells were fixed and stained for SA-β-Gal for 6-hours. Cells nuclei were then stained with DAPI for total-cell enumeration. SA-β-Gal positive cells, and total cells per frame, were quantified by microscopy. For each 5-HT2A agonist assessed, the % of senescent cells is expressed relative to the hyperglycemic control. In addition, the Fold reduction in cell senescence relative to hyperglycemic controls are also shown. NS=Not significant; * is significant to relative p value of number of stars.

FIG. 111 shows non-limiting, exemplary data for TCI-2. Effects of specific phenethylamine 5-HT2A receptor agonists on reduction of Hyperglycemia induced Disease Promoting Cellular Senescence: Inhibition of Cell Senescence and Disease Processes are Drug and Dose Dependent. ARPE-19 cells in physiologically relevant media (HPLM; 7% FCS 5 mM Glucose) were seeded overnight prior to being replaced with starvation media (HPLM; 0.5% FCS; 5 mM Glucose) containing 1 uM, 100 nM, 10 nM, 1 nM, 0.1 nM, or no drug concentrations. After overnight incubation in starvation/drug media, cells were grown in diabetic conditions (HPLM; 7% FCS; 30 mM Glucose) with 1 uM, 100 nM, 10 nM, 1 nM, 0.1 nM, or no drug concentrations for 72-hours. After the 72-hour incubation, cells were fixed and stained for SA-β-Gal for 6-hours. Cells nuclei were then stained with DAPI for total-cell enumeration. SA-β-Gal positive cells, and total cells per frame, were quantified by microscopy. For each 5-HT2A agonist assessed, the % of senescent cells is expressed relative to the hyperglycemic control. In addition, the Fold reduction in cell senescence relative to hyperglycemic controls are also shown. NS=Not significant; * is significant to relative p value of number of stars.

FIG. 112 shows non-limiting, exemplary data for TCE-2. Effects of specific phenethylamine 5-HT2A receptor agonists on reduction of Hyperglycemia induced Disease Promoting Cellular Senescence: Inhibition of Cell Senescence and Disease Processes are Drug and Dose Dependent. ARPE-19 cells in physiologically relevant media (HPLM: 7% FCS; 5 mM Glucose) were seeded overnight prior to being replaced with starvation media (HPLM; 0.5% FCS; 5 mM Glucose) containing 1 uM, 100 nM, 10 nM, 1 nM, 0.1 nM, or no drug concentrations. After overnight incubation in starvation/drug media, cells were grown in diabetic conditions (HPLM; 7% FCS; 30 mM Glucose) with 1 uM, 100 nM, 10 nM, 1 nM, 0.1 nM, or no drug concentrations for 72-hours. After the 72-hour incubation, cells were fixed and stained for SA-β-Gal for 6-hours. Cells nuclei were then stained with DAPI for total-cell enumeration. SA-β-Gal positive cells, and total cells per frame, were quantified by microscopy. For each 5-HT2A agonist assessed, the % of senescent cells is expressed relative to the hyperglycemic control. In addition, the Fold reduction in cell senescence relative to hyperglycemic controls are also shown. NS=Not significant: * is significant to relative p value of number of stars.

FIG. 113 shows non-limiting, exemplary data for 2C-NP. Effects of specific phenethylamine 5-HT2A receptor agonists on reduction of Hyperglycemia induced Disease Promoting Cellular Senescence: Inhibition of Cell Senescence and Disease Processes are Drug and Dose Dependent. ARPE-19 cells in physiologically relevant media (HPLM; 7% FCS; 5 mM Glucose) were seeded overnight prior to being replaced with starvation media (HPLM; 0.5% FCS; 5 mM Glucose) containing 1 uM, 100 nM, 10 nM, 1 nM, 0.1 nM, or no drug concentrations. After overnight incubation in starvation/drug media, cells were grown in diabetic conditions (HPLM; 7% FCS; 30 mM Glucose) with 1 uM, 100 nM, 10 nM, 1 nM, 0.1 nM, or no drug concentrations for 72-hours. After the 72-hour incubation, cells were fixed and stained for SA-R-Gal for 6-hours. Cells nuclei were then stained with DAPI for total-cell enumeration. SA-D-Gal positive cells, and total cells per frame, were quantified by microscopy. For each 5-HT2A agonist assessed, the % of senescent cells is expressed relative to the hyperglycemic control. In addition, the Fold reduction in cell senescence relative to hyperglycemic controls are also shown. NS=Not significant; * is significant to relative p value of number of stars.

FIG. 114 shows a representative Western Blot. Retinal Pigmented Epithelial Cells Express Serotonin 2A Receptors Targeted by 5-HT2A agonist Therapeutics. Western blot analysis of 5-HT2 receptor expression in 293 cells overexpressing the serotonin 2A receptor (positive control for 2A expression), ARPE-19 cells established from adult RPE, and primary RPE. Cells were grown, and lysates were gathered. Lysates were normalized to protein concentration prior to running SDS-PAGE and western blot analysis using antibodies specific to the 5-HT2A receptor, 5-HT2B receptor, or 5HT-2C receptor, as well as a b-actin protein loading control.

### DETAILED DESCRIPTION OF THE INVENTION

Detailed descriptions of one or more embodiments are provided herein. It is to be understood, however, that the invention can be embodied in various forms. Therefore, specific details disclosed herein are not to be interpreted as limiting, but rather as a basis for the claims and as a representative basis for teaching one skilled in the art to employ the invention in any appropriate manner.

The singular forms “a”, “an” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one.” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example. “a process involving steps a, b, and c” means that the process includes at least steps a, b and c. Wherever the terms “a” or “an” are used, “one or more” is understood, unless such interpretation is nonsensical in context.

As used herein, the term “about” can refer to approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower). In embodiments, the term “about” can be denoted by “~”.

As used herein, the term “substantially the same” or “substantially” can refer to variability typical for a particular method is taken into account.

The terms “sufficient” and “effective”, as used interchangeably herein, can refer to an amount (e.g., mass, volume, dosage, concentration, and/or time period) needed to achieve one or more desired result(s).

Before explaining at least one embodiment of the disclosure in detail, it is to be understood that the disclosure is not necessarily limited in its application to the details set forth in the following description or exemplified by the examples. The disclosure can be used for other embodiments or of being practiced or carried out in various ways. Other compositions, compounds, methods, features, and advantages of the disclosure will be or become apparent to one having ordinary skill in the art upon examination of the following drawings, detailed description, and examples. All such additional compositions, compounds, methods, features, and advantages can be included within this description, and be within the scope of the disclosure.

Aspects of the invention are drawn towards method of treating or preventing senescence, mitochondrial dysfunction, or a disease or disorder thereof, comprising administering a therapeutically effective amount of a 5-HT2A receptor agonist to a subject in need thereof, wherein the 5-HT2A receptor agonist is selected from the group consisting of:

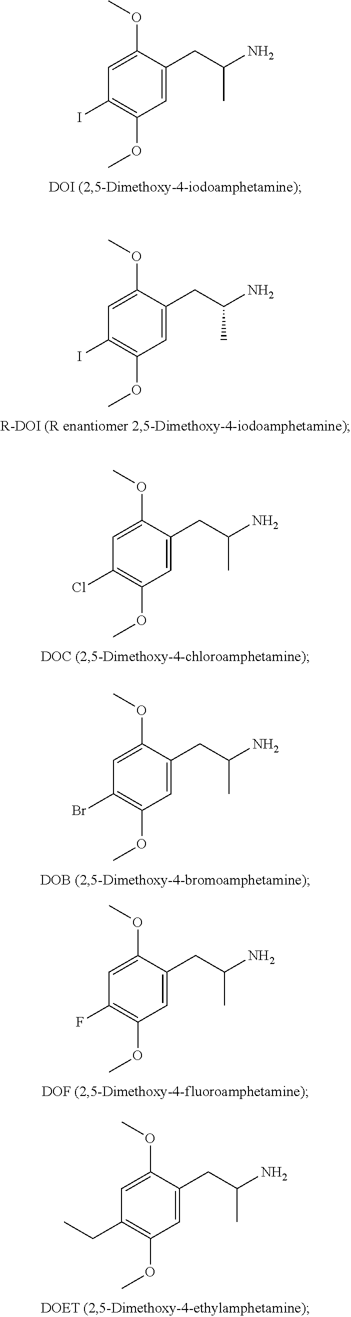

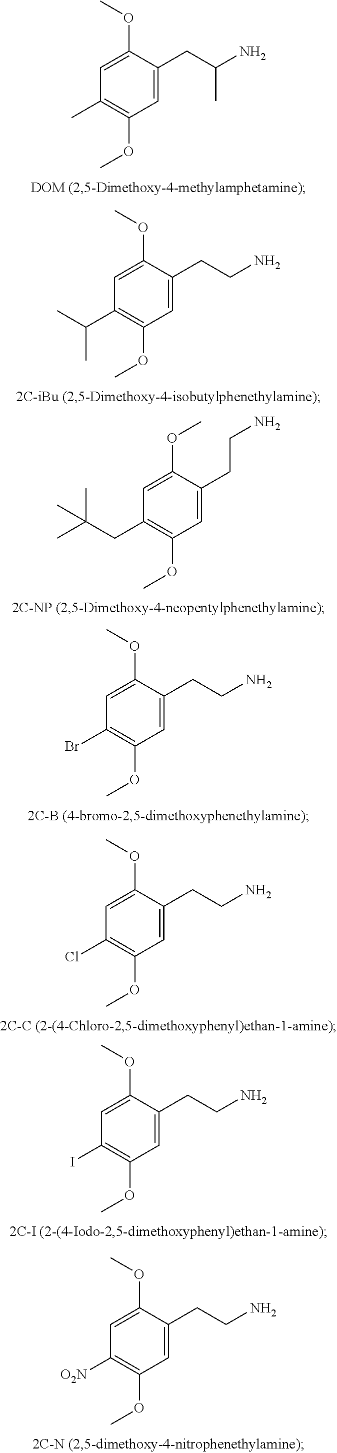

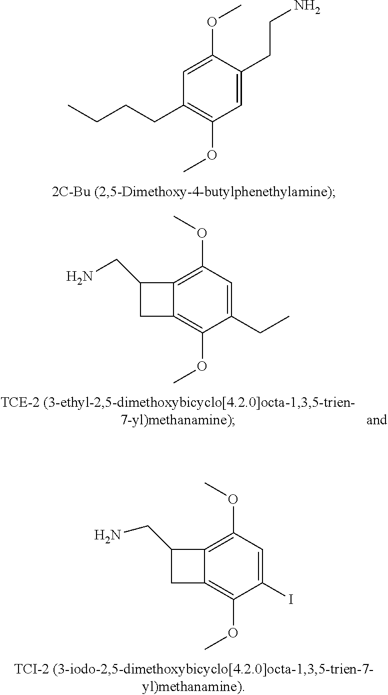

However, unexpectedly, not all phenethylamines have a therapeutic effect. For example, R-DOTFM can function as a disease exacerbator:

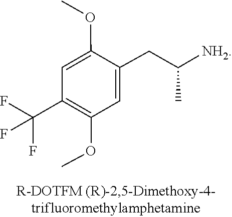 [SMILES: COc1cc(C(F)(F)F)c(OC)cc1C[C@@H](C)N]

Without wishing to be bound by theory, DOTFM can function as a disease exacerbator:

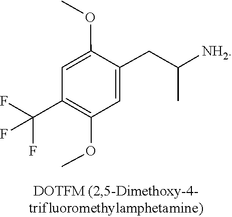 [SMILES: COc1cc(C(F)(F)F)c(OC)cc1CC(C)N]

For example, at a concentration of about 10 nM or above about 10 nM R-DOTFM and DOTFM can exacerbate disease.

As used herein, “treatment” and “treating” can refer to the management and care of a subject for the purpose of combating a condition, disease or disorder, in any manner in which one or more of the symptoms of a disease or disorder are ameliorated or otherwise beneficially altered. The term is intended to include the full spectrum of treatments for a given condition from which the patient is suffering, such as administration of the active compound for the purpose of: alleviating or relieving symptoms or complications; delaying the progression of the condition, disease or disorder; curing or eliminating the condition, disease or disorder; and/or preventing the condition, disease or disorder, wherein “preventing” or “prevention” can refer to the management and care of a patient for the purpose of hindering the development of the condition, disease or disorder, and includes the administration of the active compounds to prevent or reduce the risk of the onset of symptoms or complications. The patient to be treated can be a mammal, in particular a human being. Treatment also encompasses any pharmaceutical use of the compositions herein, for example for treating a disease as provided herein.

The term “subject” or “patient” can refer to any organism to which aspects of the disclosure can be administered, e.g., for experimental, diagnostic, prophylactic, and/or therapeutic purposes. Typical subjects to which compounds of the present disclosure can be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” can refer to a subject noted above or another organism that is alive. The term “living subject” can refer to the entire subject or organism and not just a part excised (e.g., a liver or other organ) from the living subject.

In yet another aspect, provided is a pharmaceutical composition comprising a therapeutically effective amount of the compounds described herein, or a pharmaceutically acceptable salt, prodrug, hydrate, or solvate thereof, and a pharmaceutically acceptable carrier, diluent, or excipient. The formulation or pharmaceutical compound of the present invention can be administered alone, but can also be administered with other compounds, excipients, fillers, binders, carriers or other vehicles selected based upon the chosen route of administration and standard pharmaceutical practice.

Administration can be by way of carriers or vehicles, such as injectable solutions, including sterile aqueous or non-aqueous solutions, or saline solutions; creams; lotions; capsules; tablets; granules; pellets; powders; suspensions, emulsions, or microemulsions; patches; micelles; liposomes; vesicles; implants, including microimplants; eye drops; other proteins and peptides; synthetic polymers; microspheres; nanoparticles; and the like.

In embodiments, compositions and formulations will be formulated as solutions, suspensions and other dosage forms for topical administration. Aqueous solutions are can be used, based on ease of formulation, biological compatibility (especially in view of the malady to be treated, e.g., corneal diseases and injuries), as well as a patient's ability to easily administer such compositions by means of instilling one or more drops of the solutions onto the surface of the affected eyes. However, the compositions can also be suspensions, viscous or semi-viscous gels, or other types of solid or semi-solid compositions. Suspensions can be preferred for compositions which are less soluble in water. The formulations or pharmaceutical composition of the present disclosure can also be included, or packaged, with other non-toxic compounds, such as pharmaceutically acceptable carriers, excipients, binders and fillers including, but not limited to, glucose, lactose, gum acacia, gelatin, mannitol, xanthan gum, locust bean gum, galactose, oligosaccharides and/or polysaccharides, starch paste, magnesium trisilicate, talc, corn starch, starch fragments, keratin, colloidal silica, potato starch, urea, dextrans, dextrins, and the like. For example, the pharmaceutically acceptable carriers, excipients, binders, and fillers contemplated for use in the practice of the present disclosure are those which render the compounds of the invention amenable to intravitreal delivery, intraocular delivery, ocular delivery, subretinal delivery, intrathecal delivery, intravenous delivery, subcutaneous delivery, transcutaneous delivery, intracutaneous delivery, intracranial delivery, topical delivery and the like. Moreover, the packaging material can be biologically inert or lack bioactivity, such as plastic polymers, silicone, etc. And can be processed internally by the subject without affecting the effectiveness of the composition/formulation packaged and/or delivered therewith.

In embodiments, the 5-HT2A receptor agonist is formulated as a pharmaceutical composition comprising one or more pharmaceutically acceptable excipients. For example, the pharmaceutical composition is formulated for topical administration, ocular administration, parenteral administration, intravenous administration, intranasal administration, or oral administration.

Examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. Formulations can additionally include: lubricating agents such as talc, magnesium stearate, and mineral oil; wetting agents; emulsifying and suspending agents; preserving agents such as methyl- and propylhydroxybenzoates; sweetening agents; and flavoring agents. The disclosed compositions can be formulated so as to provide quick, sustained or delayed release of the active agent(s) after administration to the patient by employing procedures known in the art.

For example, the pharmaceutical composition is formulated for ocular administration can comprise an eye drop, a contact lens, an ocular hydrogel, an intraocular implant, or intravitreal injection. In some embodiments, a disclosed pharmaceutical composition can be formulated in an ophthalmic formulation. Ophthalmic formulations of the disclosure include topical formulations, such as eye drops, gels, and ointments. Ophthalmic formulations can contain one or more viscosity-modifying agents and have a viscosity that feels comfortable to the eye and do not cause blurring of the vision. For example, an ophthalmic formulation can have a viscosity of 1.0 to 100,000 cP (e.g., from 2.0 to 90,000 cP or from 2.5 and 75,000 cP). Viscosity-modifying agents are substances that have the ability to cause thickening (increase the viscosity) of ophthalmic formulations. Viscosity modifying agents include xanthan gum, edetate, methylcellulose, carboxymethylcellulose, hydroxypropyl methylcellulose, hydroxyethyl cellulose, polyethylene glycol, propylene glycol alginate, chitosan, and tragacanth. Hydrogels can also be used as viscosity-enhancing excipients, particularly in artificial tears. Compatible viscosity-adjusting agents can be used in all formulations mentioned herein. Concentrations of viscosity-modifying agents in ophthalmic formulations of the disclosure can range from about 0.1 percent to about 10 percent by weight (e.g., between 1 percent and 5 percent by weight). Tween, Tween 80, Tween80/polysorbate80, polysorbate, or similar compositions can be used to enhance solubilization and increase ocular surface area. Sorbitol can be used as a combined tonicity-adjusting and viscosity-modifying excipient. Sorbitol can be used in ophthalmic formulations of the disclosure in a concentration range from about 0.1 to about 10 percent (e.g., from 2 percent to 5 percent by weight).

Additional ophthalmic formulations of the disclosure include contact lenses. In some embodiments, a disclosed compound or pharmaceutical composition is incorporated into a contact lens for ocular drug delivery. The contact lens can be a hydrogel contact lens or a molecularly imprinted contact lens.

One advantageous route of administration is topical administration. As used herein, the term “topical administration” can refer to administration onto any accessible body surface of any human or animal species, preferably the human species, for example, such as to the surface of the eye. Accordingly, the term “topical pharmaceutical composition” can include those dosage forms in which the compound is administered externally by direct contact with a topical treatment site, for example, the eye or the skin. The term “topical ocular pharmaceutical composition” can refer to a pharmaceutical composition suitable for administration directly to the eye. The term “topical epidermal pharmaceutical composition” can refer to a pharmaceutical composition suitable for administration directed to the epidermal layer of the skin, for example, the eyelid, the eyebrow, the scalp or the body.

In some embodiments, the pharmaceutical composition is provided in unit dosage form. In some such embodiments, the compound is in a total amount of between 0.1 and 100 mg, or between 1 and 10 mg. In some embodiments, the compound is in a total amount of between 0.1 and 1 mg, or between 0.5 and 5 mg. In some embodiments, the unit dosage form of the pharmaceutical composition is an immediate release, controlled release, sustained release, extended release, or modified release formulation.

The term “therapeutically effective amount” as used herein can refer to that amount of an embodiment of the composition or pharmaceutical composition being administered that will relieve to some extent one or more of the symptoms of the disease or condition being treated, and/or that amount that will prevent, to some extent, one or more of the symptoms of the condition or disease that the subject being treated has or is at risk of developing.

A therapeutically effective dose can depend upon a number of factors known to those of ordinary skill in the art. The dosage can vary depending upon known factors such as the pharmacodynamic characteristics of the active ingredient and its mode and route of administration: time of administration of active ingredient; identity, size, condition, age, sex, health and weight of the subject or sample being treated; nature and extent of symptoms; kind of concurrent treatment, frequency of treatment and the effect desired; and rate of excretion. These amounts can be readily determined by the skilled artisan.

A surprising aspect of this disclosure are the low, therapeutically effective doses of the compounds described herein. For example, without wishing to be bound by theory, compounds described herein can possess an inverse dose response curve with the lowest doses being more effective than higher concentrations.

In embodiments, the therapeutically effective amount comprises about about 1 uM, less than about 1 uM, about 100 nM, about 10 nM, about 1 nM, about 0.1 nM, or less than 0.1 nM. For example, the therapeutically effective amount comprises about 1 uM, about 900 nM, about 800 nM, about 750 nM, about 700 nM about 600 nM, about 500 nM, about 400 nM, about 300 nM, about 200 nM, about 250 nM, about 150 nM, about 100 nM, about 75 nM, about 50 nM, about 40 nM, about 30 nM, about 25 nM, about 20 nM, about 15 nM, about 10 nM, about 9 nM, about 8 nM, about 7 nM, about 6 nM, about 5 nM, about 4 nM, about 3 nM, about 2 nM, about 1 nM, about 0.75 nM, about 0.60 nM, about 0.50 nM, about 0.40 nM about 0.30 nM, about 0.25 nM, about 0.20 nM, about 0.15 nM, about 0.10 nM, about 0.09 nM, about 0.08 nM, about 0.07 nM, about 0.06 nM, about 0.05 nM, about 0.04 nM, about 0.03 nM, about 0.025 nM, about 0.02 nM, about 0.015 nM, about 0.01 nM, about 0.009 nM, about 0.008 nM, about 0.007 nM, about 0.006 nM, about 0.005 nM, about 0.004 nM, about 0.003 nM, about 0.0025 nM, about 0.002 nM, about 0.0015 nM, about 0.001 nM, about 0.00075 nM, about 0.0005 nM, about 0.00025 nM, about 0.0002 nM, about 0.00015 nM, about 0.00010 nM, about 0.00001 nM, about 0.000001 nM or less than about 0.0000010 nM.

For example, the therapeutically effective amount of R-DOI can comprise about 10 nM to about 0.0001 nM or less than 0.0001 nM. For example, the therapeutically effective amount of TCE-2 can comprise about 100 nM. For example, the therapeutically effective amount of TCI-2 can comprise about 1 uM, less than about 1 uM, about 100 nM, about 10 nM, about 1 nM, about 0.1 nM, or less than 0.1 nM. For example, the therapeutically effective amount of 2C-NP can comprise about 0.1 nM or less. In embodiments, the therapeutically effective amount of a compound described herein comprises greater than about 1 uM, about 1 uM, less than about 1 uM, about 100 nM, about 10 nM, about 1 nM, about 0.1 nM, about 0.05 nM, about 0.001, about 0.0001 nM, about 0.00001 nM, and less than 0.00001 nM. For example, the therapeutically effective amount of R-DOI can comprise about 10 nM to less about 0.001 nM. In embodiments, the therapeutically effective amount of TCE-2 can comprise about 100 nM. In embodiments, the therapeutically effective amount of TCI-2 can comprise about 1 uM, less than about 1 uM, about 100 nM, about 10 nM, about 1 nM, about 0.1 nM, about 0.05 nM, about 0.001, and less than 0.001 nM. In embodiments, the therapeutically effective amount of 2C-NP can comprise about 0.1 nM, about 0.01 nM, about 0.001 nM, about 0.0001 nM, or less than 0.1 nM. In embodiments, the therapeutically effective amount of 2C-B, 2C-C, 2C-I, 2C-Bu, or 2C-N can comprise about 0.1 nM, about 0.001 nM, about 0.0001 nM, or less than 0.1 nM. In embodiments, the therapeutically effective amount of DOI can comprise about 10 uM to about 50 uM.

Aspects of the invention are drawn towards treating a senescence, a senescence disease or disorder, mitochondrial dysfunction, and/or a mitochondrial dysfunction disease or disorder.

Mitochondrial dysfunction can refer to a loss of efficiency in the electron transport chain and reductions in the synthesis of high-energy molecules, such as adenosine-5′-triphosphate (ATP). Mitochondrial dysfunction can also refer to an increase in disease promoting production of reactive oxygen species. It is a characteristic of aging, and many chronic diseases. These diseases comprise neurodegenerative diseases, cardiovascular diseases, diabetes and metabolic syndrome, autoimmune diseases, neurobehavioral and psychiatric diseases, gastrointestinal disorders, musculoskeletal diseases, cancer, and chronic infections.

Cellular senescence can refer to a form of cellular aging linked to biological aging and disease development and progression. Cellular senescence is a proinflammatory cell fate associated with age-related diseases, including Alzheimer's disease (AD) and Age-Related Macular Degeneration (AMD). The senescence phenotype expresses in cells undergoing terminal, replicative arrest that displays cell enlargement, chromatin alterations, SASP, and cell cycle regulatory proteins (cyclins and cyclin-dependent kinases). Persistent accumulation of senescence is associated with age-related diseases and functional decay. The clearance of senescent cells from tissue alleviates pathologies related to aging because they propagate degenerative and proinflammatory events in their microenvironment.

In embodiments, the senescence disease or disorder comprises a neurodegenerative disease, a metabolic disorder, lysosomal dysfunction, protein aggregative diseases, a psychiatric condition, an inflammatory or autoimmune disease, or an aging related disorder.

In embodiments, the psychiatric condition can comprise psychiatric conditions known in the art. For example, the psychiatric condition can comprise depression and addiction.

For example, the neurodegenerative disease comprises dementia, Alzheimer's disease, Parkinson's disease, Huntington's disease, dementia, cognitive impairment, and motor neuron dysfunction. In embodiments, the metabolic disorder comprises type II diabetes, obesity, hyperglycemia, glucose intolerance, insulin resistance (i.e., hyperinsulinemia, metabolic syndrome, syndrome X), hypercholesterolemia, hypertension, hyperlipoproteinemia, hyperlipidemia (e.g., dyslipidemia), hypertriglylceridemia, cardiovascular disease, atherosclerosis, peripheral vascular disease, kidney disease, ketoacidosis, thrombotic disorders, nephropathy, diabetic neuropathy, diabetic retinopathy, sexual dysfunction, dermatopathy, dyspepsia, hypoglycemia, cancer or edema.

In embodiments, the inflammatory or autoimmune disease comprises celiac disease, vasculitis, lupus, chronic obstructive pulmonary disease (COPD), atherosclerosis, arthritis, atopic dermatitis, psoriasis, vitiligo, keratitis, allergic conjunctivitis, retinitis, uveitis, dry eye, osteoarthritis, osteoporosis, rheumatoid arthritis, oral mucositis, inflammatory bowel disease, kyphosis, and herniated intervertebral disc.

In embodiments, the aging related disorder comprises renal disease, renal failure, frailty, hearing loss, muscle fatigue, skin conditions, skin wound healing, liver fibrosis, pancreatic fibrosis, oral submucosa fibrosis, sarcopenia, cancer, dementia, cardiovascular disease, such as arteriosclerosis, hypertension, diabetes mellitus (type I or type II) arthritis, cataracts, diabetic retinopathies, retinopathies, psychiatric conditions such as depression and addiction, Alzheimer's disease, Parkinson's disease, age-related macular degeneration (AMD), and osteoporosis.

Additional aspects of the invention are drawn towards a method of identifying a subject responsive to a 5-HT2A receptor agonist described herein for the treatment or prevention of senescence, mitochondrial dysfunction, lysosomal dysfunction, protein aggregative diseases, or a disease or disorder thereof. In embodiments, the method comprises determining if the subject meets one or more of the following criteria: (1) at or above the transition age of ocular disease development; (2) a blood glucose level greater than or equal to about 6 mmol/L; (3) presents with lipofuscin and drusen deposition; (4) presents with autofluorescent oxidized flavoproteins; (5) an Early Treatment Diabetic Retinopathy Study (ETDRS) disease severity score of about 20 or greater; and/or (6) presents with a retinopathy. For example, the transition age of ocular disease development can comprise about 50 years old to about 60 years old in a human subject.

### EXAMPLES

Examples are provided herein to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Targeting Serotonin 5-HT2A Receptor for Treatment of Aging-Associated Pathologies

### Abstract

An embodiment of the innovation described herein is the role of serotonin 5HT2A receptors in aging-associated disease processes and cell senescence. Specifically, we have shown that upon deletion of 5HT2A receptors from retinal epithelial cells, cells display an aged phenotype. This phenotype is thought to evolve as removal of 5HT2A disrupts mitochondrial function and elicits development of intracellular oxidative stress. These mitochondrial and oxidative stress processes are suppressed by compounds that act as agonists to the serotonin 5HT2A receptor. As the senescent phenotype progresses, cell morphology becomes disrupted, stress fibers form, and cells begin to exit their normal cell cycle. Moreover, cells fail to appropriately process proteins, and aggregates of misfolded proteins form within the cytoplasm of cells. Consequently, this results in activation of chronic inflammatory processes, including expression of inflammatory cytokines. VEGF. IL-6 and IL1b.

Through these processes, the presence of cells with this senescent phenotype is multiplied and spread to adjacent cells. The phenotype of these retinal epithelia cells is reminiscent of what is present in the retinal epithelia during age-related macular degeneration (AMD). In addition, these collective processes are common in most aging-associated pathologies. Diseases that have these phenotypes are wide ranging, but include, Parkinson's (protein aggregation in Lewy's bodies, mitochondrial dysfunction, etc), Alzheimer's (protein aggregation and misfolding; inflammation), AMD (cell senescence, stress fibers, in wet AMD-VEGF expression/vascularization), diabetic retinopathies, kidney disorders, cardiac disorders, development of cancers etc. Identification of druggable targets, like the 5HT2A receptor, that can disrupt these processes is paramount in understanding how to either reverse or prevent development of aging-associated diseases.

Our recent identification of serotonin and specifically 5HT2A receptors affecting aging-related disease processes, including development of cell senescence, provides a druggable target for modulating a number of adverse disease outcomes and chronic pathologies. In today's aging population, these disease targets are numerous, including diseases of the eyes, lungs, kidneys, brain, liver, and heart. For example, clinical trials are underway for other potential senolytic drugs, which target completely different processes, for diabetes, idiopathic pulmonary fibrosis, Alzheimer's, Parkinson's, osteoarthritis, cancer, and ocular diseases. Recent discovers of the contributions of cell senescence to mitochondrial dysfunction, oxidative stress and chronic inflammation beacons for the identification and development of drugs that can disrupt these processes. In this regard, the 5HT2A receptor is a viable druggable target that can resolve these aging disease processes.

To date, serotonin receptors have not been associated with aging associated diseases. As such, identifying the role of this receptor in these processes provides a unique and new target. Moreover, there are a plethora of new drugs that are being developed that target the 5HT2A receptor for modulation. This class of compounds can have drug-like properties making them particularly suited for reaching and modulating 5HT2A receptor function. Senolytic drugs, which are meant clear the cell senescent phenotype, are an emerging drug class that are beginning to exhibit immense potential in aging associated diseases. Some of these compounds have shown efficacy in over 40 disease states. However, not all of these drugs are capable of selectable penetration into tissues or organs (ie. Eye and brain) at concentrations to be effective at reverting the phenotype; whereas effective penetration of 5HT2A receptor targeting drugs into these tissues at the identified therapeutically active levels has been demonstrated.

### Example 2

### Role of the Serotonin 2A Receptor (5-HT2AR) in Retinal Pigmented Epithelial Cell-Associated Development and Progression of Age-Related Macular Degeneration

Age-related macular degeneration (AMD) is the leading cause of blindness in elderly with accelerated progression in diabetic individuals. AMD is characterized by degeneration of the retinal pigmented epithelium (RPE) through increased ROS, inflammation, drusen deposition, metabolic dysregulation, and persistence of senescent cells. Although AMD pathology is defined, molecular mechanisms underlying its development and progression remain poorly understood. Serotonin, partially through the serotonin 2A receptor (5-HT2AR), is essential for retinal development and health by regulating cellular metabolism, insulin sensitivity, vasculature, and inflammation. We have shown that expression of retinal 5-HT2AR decreases with age: however, its role in RPE function is unknown. We therefore interrogated the role of 5-HT2AR in RPE function and AMD-associated disease processes.

Morphological and functional consequences of 5-HT2AR modulation were assessed in ARPE-19 cells via pharmacological agonism/antagonism (R-DOI/M100,907) or CRISPR/Cas9 5-HT2AR-specific knockout. Cell morphology, lysosomal content, and cytoskeletal structures were evaluated by fluorescent microscopy and flow cytometry. Metabolic dysfunction was evaluated in 5-HT2AR knockouts or in wildtype cells grown in physiological and pathological glucose concentrations by quantification of intracellular ROS and western analyses of antioxidant protein expression. Senescence-associated D-galactosidase (SA-β-Gal) expression was evaluated by microscopy.

Inhibition of 5-HT2AR expression/function resulted in large cell morphology, extensive cytoskeletal stress fibers, and decreased proliferation. Knockouts also exhibited increased granularity, autofluorescence, ROS levels, and lysosomal biomass containing active SA-3-Gal, indicative of a pre-senescent phenotype. Conversely, 5-HT2AR agonism suppressed pathological glucose-mediated ROS production and SA-3-Gal expression, while increasing antioxidant protein expression.

Decreased expression or function of 5-HT2AR within RPE, as observed in aging retinas, is associated with age-related cellular disease processes that are hallmarks of AMD. Disease-promoting attributes such as cellular morphology, lysosomal activity, metabolism, and senescence are modulated via 5-HT2AR. Intriguingly. 5-HT2AR pharmacological agonism inhibited pathological processes associated with AMD, and therefore can be a new therapeutic target.

### Example 3

### The Role of Serotonin and the 5-HT2A Receptor in Ocular Disease: a Through the Looking Glass View of the Therapeutic Potential of Psychedelics in Inflammation- and Aging-Associated Disease Processes

### Background

Age-related eve diseases, such as age-related macular degeneration, diabetic retinopathy, and glaucoma, are the leading causes of blindness and low vision in the United States. In addition, ocular infections by viruses and bacteria are worldwide major contributors to vision loss, as well as being associated with epidemic outbreaks of conjunctivitis and keratitis. Despite differences in each of these diseases, there are many common underlying contributors to their associated pathogenesis, including inflammation, oxidative stress, metabolic dysfunction, fibrosis, neuronal damage, and vascularization. The 5-HT2A receptor (5-HT2AR) is prominently expressed within various cell types of the eye that are involved in the manifestations of ocular disease. We and others have shown that activation of the 5-HT2AR, using low dose psychedelics, suppresses many of the pathogenic processes involved in ocular disease development and progression. We therefore evaluated the therapeutic potential of psychedelics in various in vitro and in vivo inflammation- and aging-associated ocular disease models.

### Methods

The expression levels of components of 5-HT2AR-associated serotonergic pathways were assessed either in cells isolated from the eye or from in vivo ocular models and correlated to age- and inflammation-associated clinical disease. Ocular tolerance, safety and the effects of 5-HT2AR pharmacological agonism by various phenethylamines applied topically to the eye were evaluated in normotensive rabbits and rabbit ocular models of herpetic keratitis by clinically scoring ocular tolerance, intraocular pressure, and inflammation-associated clinical disease parameters. The in vitro consequences of 5-HT2AR knockout or pharmacological activation/inhibition on critical ocular cell structure and function were determined under normal or pathological glucose conditions.

### Non-Limiting Results

In ocular disease conditions, upregulation of components of the serotonergic system and serotonin levels within the aqueous humor were found to positively correlate with disease severity. By contrast, 5-HT2AR expression in the retinal pigmented epithelium (RPE) isolated from healthy eyes exhibited a significant negatively associated Pearson correlation with increasing age (r=−0.7222; p=0.0053). In addition, 5-HT2AR expression levels were significantly reduced at the age of onset of many age-related ocular diseases. Consistent with these findings. RPE cells specifying a knockout of 5-HT2AR or treated with a 5-HT2AR-specific pharmacological antagonist exhibited cellular structural and functional changes consistent with many age-related eye diseases, including, increased cell size, cytoskeletal alterations, stress fiber formation, loss of tight-junction complexes, increased oxidative stress, and a pre-senescent phenotype. Topical ocular application of selective 5-HT2 agonists were well tolerated with minimal observed behavioral effects. Upon administration, intraocular pressure in normotensive rabbit eyes was lowered 15-30% with some phenylethylamines exhibiting a duration of action as long as 7 days following a single dose. In a herpetic eye infection model, 5-HT2AR agonism reduced viral replication, neuronal reactivation, inflammation-associated clinical disease severity, neurological disease, and death.

### Non-Limiting Conclusions

Collectively, our findings support further pre-clinical development of topically applied 5-HT2A receptor agonists to suppress disease promoting inflammation- and aging-associated ocular disease processes, which contribute to development and progression of age-related macular degeneration, diabetic retinopathy, herpetic keratitis and glaucoma.

### Example 4

### Non-Limiting Summation of Embodiments Described Herein

The overall goal of these preliminary studies was to determine if retinal epithelia expressed 5-HT2A receptors and the consequences of knocking down 5-HT2A receptor expression in the retinal epithelial cell-line, ARPE-19. These foundational assessments were undertaken in part to address why systemically delivered 5-HT2A receptor agonists exhibited a predilection for retinal localization and if expression of 5HT2A receptor on these cells represented a potential target for therapeutic modulation. Our findings suggest that the retina expresses abundant 5-HT2A receptors and that its overall expression is important for proper retinal function. Furthermore, it can indicate that retinal 5-HT2A receptors possess druggable potential for resolution of 5-HT2A-associated retinal disease processes, especially those of aging. Lastly, knocking down 5-HT2A receptor expression could provide insights into its functions within the retina or its role in development of retinal disease processes.

Non-Limiting innovations described herein
Using a CRISPR-CAS9 targeted 5-HT2A deletion system
ARPE-19 cells with significant knockdown of 5-HT2A receptor expression were isolated by 2 methods: FACS-sorting of individual cells and limiting dilution of FACS sorted cells. Where possible, FACS-sorted individual 5-HT2A knockdown (KD) cells were grown up without selection and frozen back at various passages. Isolation of individual 5-HT2A KD cell-lines by limiting dilution was accomplished by transferring in a puromycin-GFP gene cassette during CRISPR/CAS9 knockout that marked 5-HT2A knockouts and aided in selecting of purified homogeneous cell lines.
Systems were developed to quantify 5-HT2A receptor expression within ARPE-19 cells. Despite seeding over 500 individual cell isolates that had taken up the 2A knockout system, we were unable to isolate ARPE-19 cells completely deleted for 5-HT2A receptor expression, suggesting that this receptor is either essential or has functions that greatly enhance cell viability and replication. However, multiple 5-HT2A cell lines that exhibited 5-HT2A receptor knockdown were isolated.
As expected, we determined that 5-HT2A receptor was expressed in both ARPE-19 cells, as well as primary isolates of human retinal epithelia cells. The expression levels of 5-HT2A in cell-line isolates knocked down for 5-HT2A receptor expression was also determined and a subset of isolates were selected for further characterization. In these subsets of cells, knockdown of 5-HT2A receptor expression was significant. Expression of 5-HT2A receptor expression relative to matched passage wildtype ARPE-19 cells ranged from approximately 6 to 36%.
From the beginning of the attempted isolation of knockouts, ARPE-19 5-HT_{2A} receptor knock down cells exhibited a profound change in cell morphology. The cells appeared to be drastically larger than controls and exhibited a flattened appearance. Isolated KD cell lines all had this phenotype, to varying degrees. Upon further assessment of select isolates, both adhered and in suspension KD cell lines were significantly enlarged compared to WT control. This phenotype is reminiscent to retinal epithelial cells in aged eyes.
All 5HT2A KD cell lines exhibited altered cytoskeletal distribution that was reminiscent of cytoskeletal changes in RPE within eyes that exhibit age-related macular degeneration (AMD) or other aged-related degenerative retinal diseases. These cytoskeletal changes included distribution of actin from the cell periphery, loss of highly regular honeycomb structure, irregular cellular size and structure, fragmented breaks in peripheral actin and most notably, the presence of abundant stress fibers.
All 5HT2A KD cell lines overexpressed phosphorylated myosin light chain (pMLC) that was highly associated with actin stress fibers. The tension on these stress fibers containing pMLC was readily apparent in the morphology of the retinal epithelial cells and resulted in disruption of intercellular tight junctional complexes that are responsible for retinal barrier integrity, a critical function of the RPE that serves as a blood brain barrier of the eye. Stress fiber activation and loss of junctional complexes contributes to edema formation. We previously noted in herpetic keratitis experiments that 5HT2A agonists inhibited the presence of edema and disruption of corneal architecture. It was also noted that WT ARPE-19 cells cultured in media that contained hyperglycemic levels of glucose showed increased phosphorylation of MLC that was inhibited by low dose 2C-iBu treatment. This is a typical readout for drugs that target ROCK activation.
5HT2A KD cells exhibited slower growth kinetics and a higher percentage of cells localized within either G1 or G2 arrest of the cell cycle. This is consistent with cells that are displaying a pre-senescent or senescent phenotype. In congruence with these findings, 5HT2A KD cells had reduced production of ATP that was consistent with mitochondrial dysfunction. By contrast, 5HT2A agonists enhanced ATP production and mitochondrial membrane potential of ARPE-19 WT cells.
5HT2A KD cells were highly granular that could be observed by sidescatter on FACS. Furthermore, they increased in autofluorescence due to the increased presence of lipofuscin, a mediator of aged disease progression. Within the granules SA-b-galactosidase was detected, a marker of pre-senescent/senescent cells and a condition that is present in many diseases of aging.
Consistent with these findings, preliminary results from 5HT2A KD cells demonstrated that in the absence of 5HT2A receptor, supernatants contained many inflammatory cytokines, including the main contributors to ocular pathologies—IL-6 and VEGF. These cytokines contribute to disease progression by inducing chronic inflammatory processes and disruption of barrier integrity. In addition, they are markers for many diseases of aging.

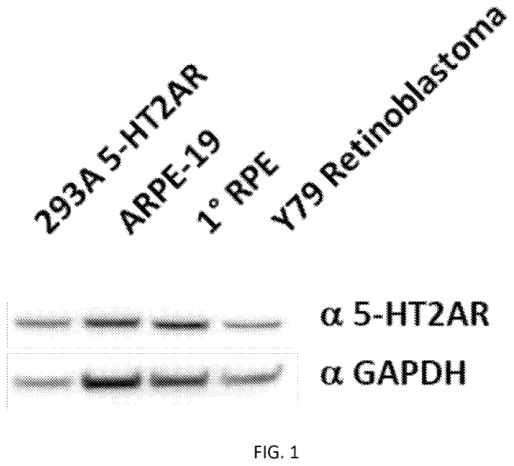

### HT2A Receptors are Expressed Both in the ARPE-19 Cell Line, in a Retinoblastoma Cancer Cell-Line, as Well as in Human Primary Retinal Epithelial Cells (FIG. 1).

To determine if 5-HT2A receptors were expressed in retinal epithelia, cell lysates were generated from ARPE-19 cells obtained from ATCC or isolated primary human retinal epithelia cells obtained from Sciencell. Cell lysates were also obtained from 293 cells engineered to express 5-HT2AR and a retinoblastoma cell line, Y79. Expression of 5-HT2A receptor was visualized by western using an anti-human 5-HT2A receptor antibody from SCBT, as we have described previously. All retinal derived cells exhibited 5-HT2A receptor expression.

CONCLUSION: ARPE-19 cells and primary retinal epithelia express 5-HT2A receptors and therefore have the potential to be targeted by 5-HT2A receptor agonists and antagonists.

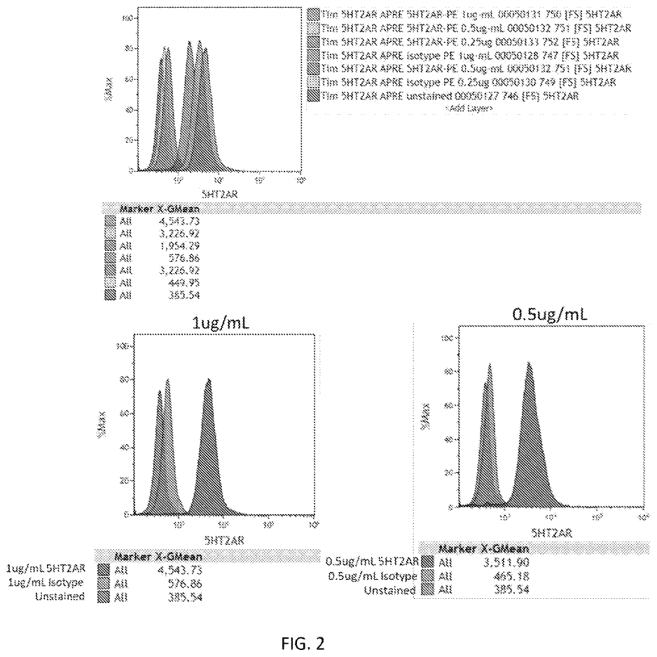

### Development and Optimization of a FACS Screening Assay for Quantitative Determination of 5-HT2A Receptor Expression in ARPE-19 Cells (FIG. 2).

Flow assays were optimized and validated for specific and quantitative detection of 5-HT2A Receptor in Retinal Epithelial Cells (ARPE-19). Below shows the concentration of antibodies utilized to detect and differentiate protein expression, as well as the mean fluorescent intensities derived from each condition. Unstained cells, or cells that were probed with isotype matched control antibodies exhibited minimal staining and were well differentiated from 5-HT2A receptor specific stained cells.

CONCLUSION: An antibody concentration of 0.5 ug/ml to 1 ug/ml was determined to give strong specific and quantifiably differentiating signal intensities. Concentrations of 5-HT2A specific antibody at 0.5 ug/ml gave slightly lower isotype background positivity; however, 1 ug/ml exhibited a higher signal to noise. Use of 0.5 ug/ml reduced antibody cost within the assays by half Importantly, the data further confirmed western analysis that 5-HT2A receptor was expressed in retinal epithelia.

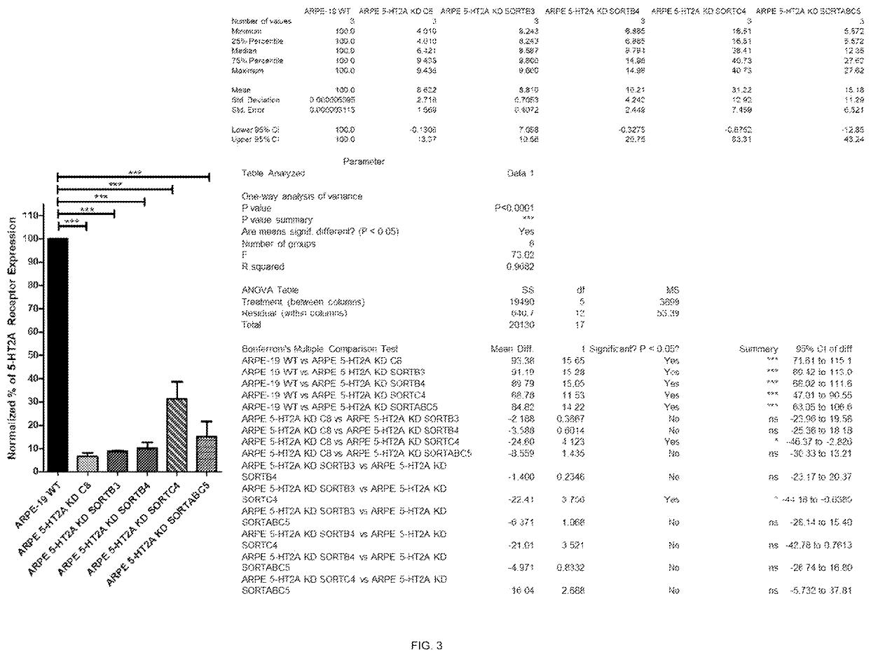

Generation of ARPE-19 Cells that are Knocked Down for 5-HT2A Receptor Expression (FIG. 3).

To generate ARPE-19 cells that were either knocked down or knocked out for 5-HT2A receptor expression we employed a CRISPR CAS9 system that specifically targeted the gene of the human 5-HT2A receptor. Following transfection of low passage ARPE-19 cells with the CRISPR CAS9 system individual cells positive for both the CAS9 and 5HT2A targeting sequence were sorted by FACS sorting into individual wells of 96 well plates. Cells were then either placed under puromycin selection or not selected. Over 500 individual cell isolates were first seeded. In addition, a heterogeneous mix of positive cells that remained after individual sorts was collected and knockout/knockdown cells were isolated by multiple rounds of limiting dilution under puromycin selection and observation of GFP positivity. In these cases, puromycin resistance and GFP expression were specifically targeted to disrupt the 5-HT2A gene by inserting within its open reading frame. Individual isolates from both conditions were propagated and analyzed for levels of 5HT2A receptor expression. A subset of these cells shown below with their relative 5-HT2A receptor expression levels were subsequently selected for extensive characterization (C8 was isolated by limiting dilution and expresses GFP; All other cell-lines below were isolated by individual cell sorting).

CONCLUSION: ARPE-19 cell-lines that express significantly reduced amounts of 5-HT2A receptor were individually isolated, grown into stocks and characterized for 5-HT2A receptor expression. We were unable to isolate cells that were completely devoid of 5-HT2A receptor expression despite screening more than 500 isolates, suggesting that 5-HT2A receptor expression conveys some replicative or survival advantage to retinal epithelial cells.

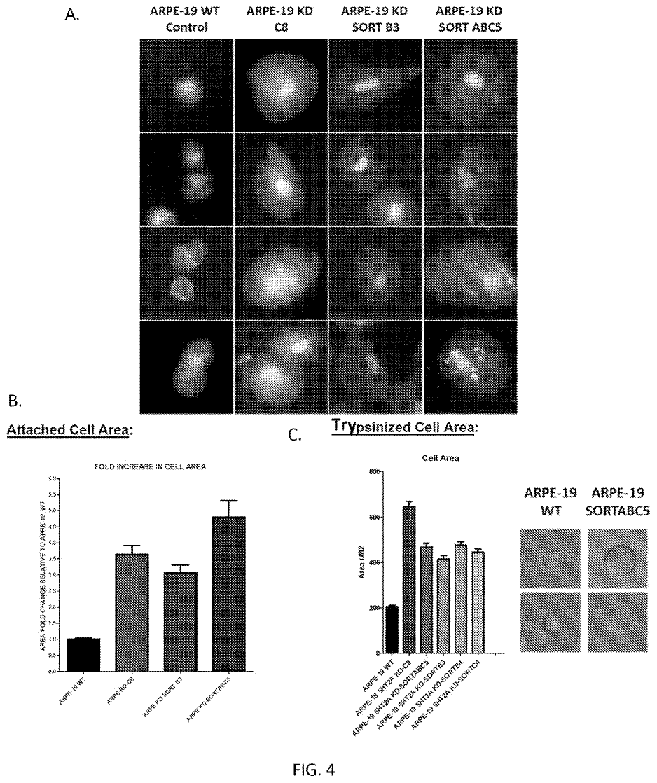

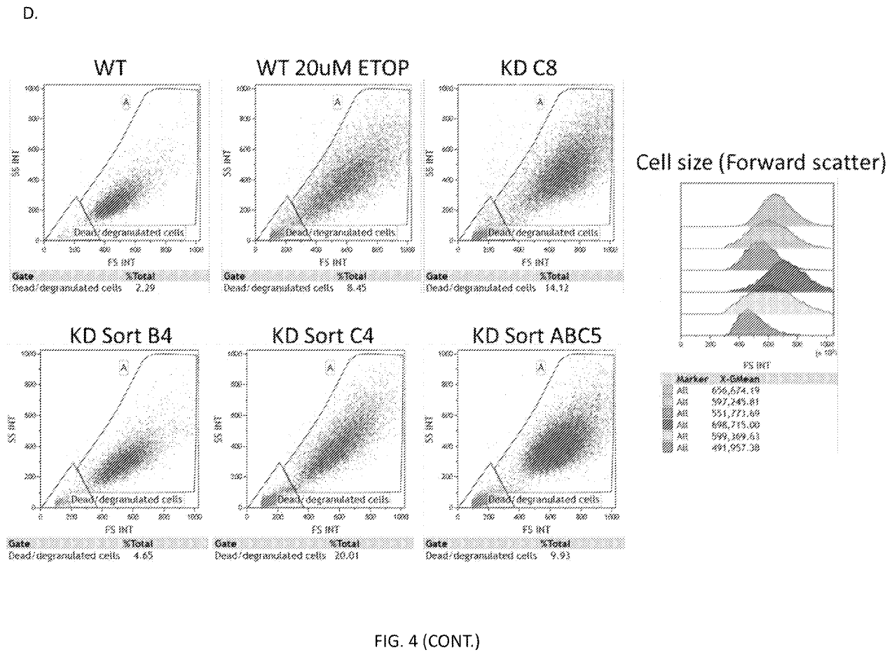

5-HT2A Receptor Knockdown Cells Exhibit a Drastically Altered Cell Morphology that is Consistent with a Pre-Senescent Phenotype and Age-Related Diseases of the Retina (FIG. 4).

Upon initial seeding of potential 5HT2A KD cells there was a readily apparent alteration of normal ARPE cell morphology. Cells appeared flattened, extended, highly attached and enlarged. Each cell line isolate maintained this enlarged and flattened cell morphology as shown to the right and quantified below. Both attached and trypsinized 5HT2A KD cell lines were significantly larger than matched ARPE-19 WT control cells. This markedly altered morphology is present in cells that have begun to undergo changes related to aging, including evolving a senescent or pre-senescent phenotype. Therefore, FACS analysis of 5HT2A KD cells was performed comparing the cell size of Forward Scatter to ARPE-19 cells, treated overnight with Etoposide, an inhibitor of topoisomerase, and a known inducer of cell senescence, and then cultured for 4 days in normal media in the absence of etoposide. As with the 5HT2A KD cells, etoposide treatment increased cell size as indicated by increased forward scatter comparisons. Further an increase in particles of “cell degranulation” sizing was also observed in all conditions compared to ARPE-19 WT controls.

The presence of enlarged cells with a pre-senescent-like phenotype following 5-HT2A Receptor knock down is reminiscent of changes in RPE morphology associated with an aged eye. Below shows RPE from young eyes versus older eves with clearly visible architectural changes to the RPE morphology, including cell enlargement and disruption of cytoskeletal networks within the cells. Staining of the RPE actin cytoskeleton with phalloidin normally can show cells with a honeycombed appearance, the actin cytoskeleton continuous and localized to the periphery of the cell, and a regular appearance of cells with homogenous size. By contrast, aged RPE exhibit an increased cell size with changes to the cytoskeletal network and architecture, including stress fiber formation.

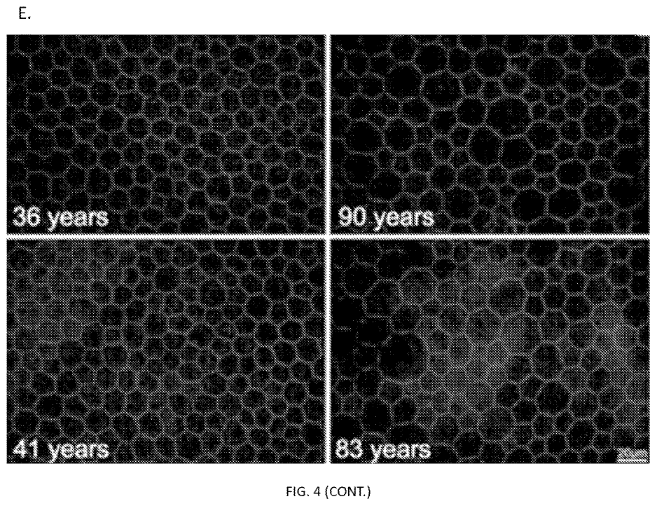

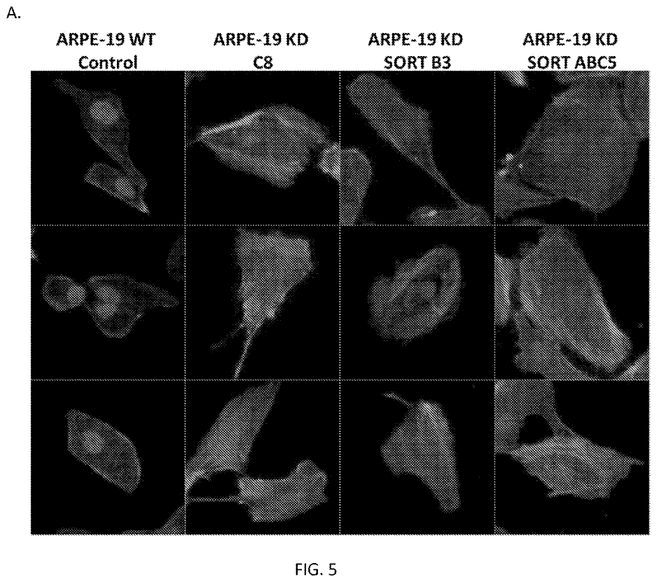

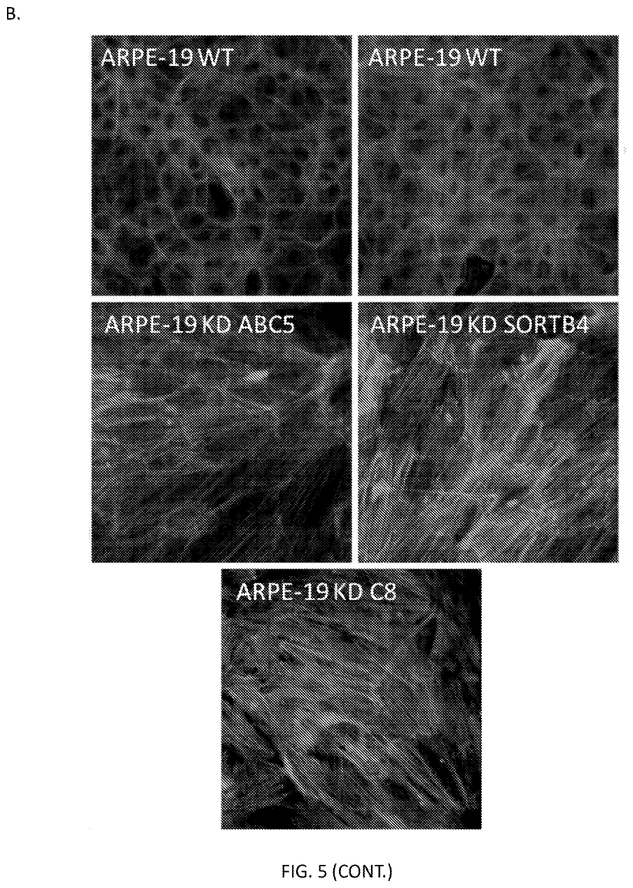

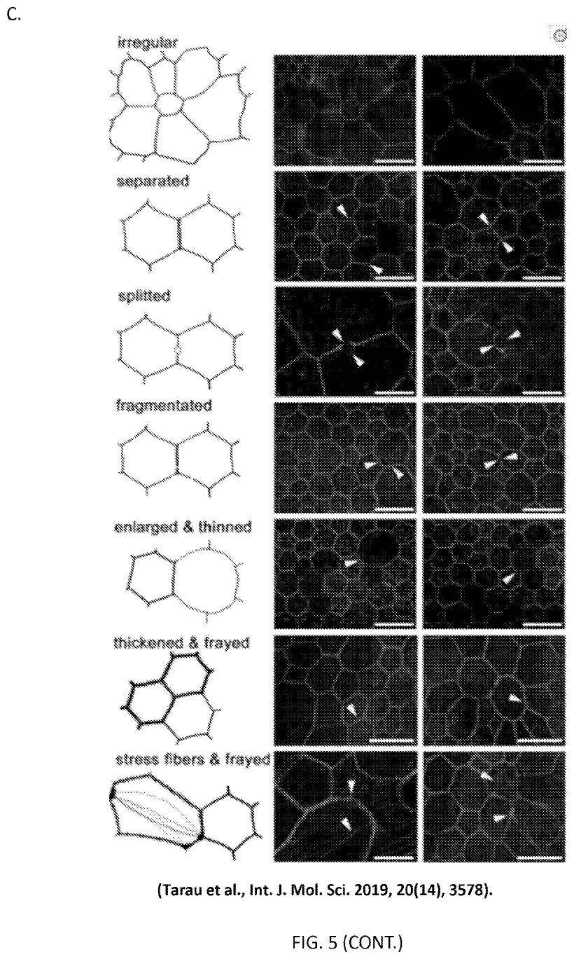

5-HT2A Receptor Knockdown RPE Cells Exhibit a Drastically Altered Cell Morphology, Redistribution and Alteration of Cytoskeletal Networks, and Stress Fiber Formation that Disrupts Cell Interactions and Barrier Integrity (FIG. 5).

Staining of the actin skeleton of sparsely plated 5HT2A KD cells showed characteristics of aged RPE. Although not done on confocal where optical sections can clearly delineate distribution, ARPE WT controls showed actin cytoskeleton that localized to the periphery of cells in continuous uninterrupted bands, normal nuclei, and absence of stress fiber formation.

By contrast, 5HT2A KD cells showed an enlarged morphology with redistribution of actin fibers from the periphery. In addition, when actin was present at the periphery, it was either thickened or disrupted.

Furthermore, most actin was present within 5HT2A KD cells within stress fibers. Interactions between RPE cells within the eye are essential for barrier integrity and retinopathies and diseases of aging generally display changes to this architecture. In this regard, when 5HT2A KD cells were cultured to confluency, the differences between these phenotypes were enhanced by the presence of prominent stress fiber formation and lack of peripheral localization. As shown in the figure on the subsequent page, collectively, these changes are consistent with cytoskeletal changes present within age-related macular degeneration (AMD).

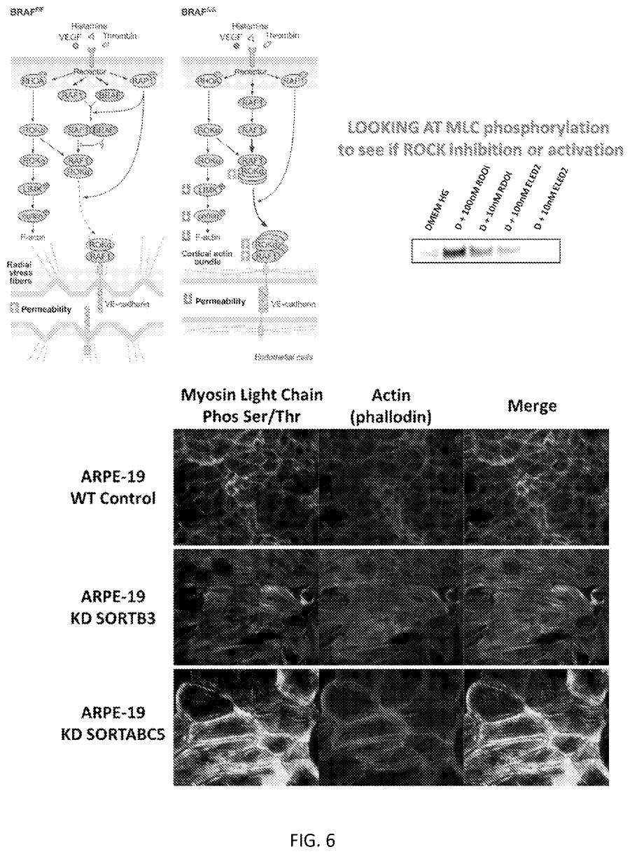

### 5-HT2A Receptor Knockdown RPE Cells and Hyperglycemic Media Activate Myosin Light Chain (MLC) Phosphorylation and Redistribution of Phosphorylated MLC to Actin Stress Fibers (FIG. 6).

Phosphorylation of myosin light chains regulate many disease processes within the eye, including ocular hypertension, allergic responses, edema formation, and RPE barrier integrity. As such, upstream processes that regulate its activation have been a major target of several drugs, including anti-inflammatories, and anti-ocular hypertensives. Phosphorylation of MLC modulates actin myosin contractility. Consequently, its activation can lead to radial stress fiber formation, loss of junctional interactions, and increased permeability with edema formation, all of which are major contributors to disease progression, especially in the RPE layers where integrity of the barrier is paramount.

We have previously provided evidence that media with high glucose levels (17.5 mM), showed phosphorylation of MLC. Further we showed that at low doses, but not high doses, of some 5HT2A agonists, including 2C-iBu, that phosphorylation of MLC was inhibited (figure to right). This can be consistent with 2C-iBu being able to regulate IOP and that 5HT2A agonists prevented edema formation and disruption of corneal architecture in a model of herpetic keratitis.

Consistent with these findings, the actin stress fiber filaments of cells knocked down for 5-HT2A receptors were studded with phosphorylated MLC potentiating actin myosin contraction and mechanical stress capable of disrupting junctional interactions and barrier integrity. Indeed, in confluent monolayers of these cells shown above, junctional interactions between cells appeared absent.

These changes can also predict alterations in endo/exocytic trafficking, a major role of the RPE and processes that are disrupted during aging-associated pathologies that contribute to disease development and progression. Indeed, consistent with these findings, in subsequent sections, exocytic and endocytic vesicle trafficking is seen to be disrupted.

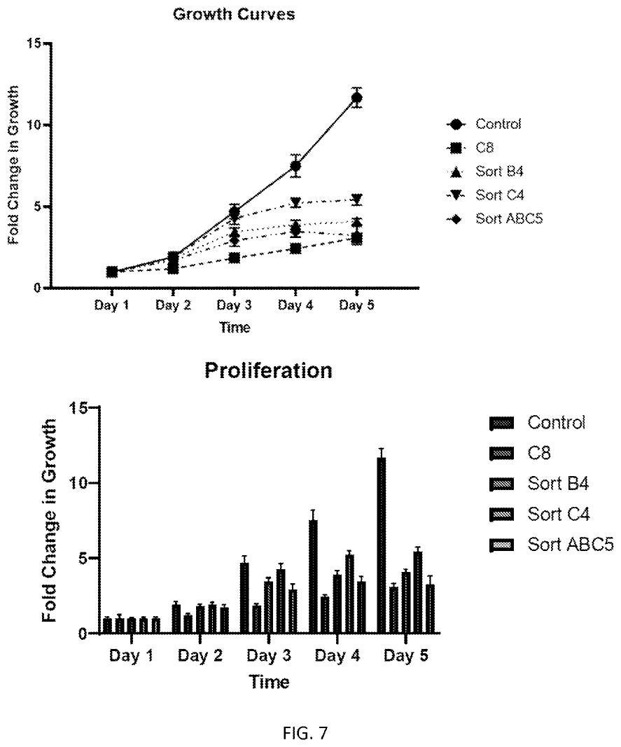

### 5-HT2A Receptor KD Cells Exhibit Slower Rates of Cellular Replication Compared to Matched WT ARPE-19 Cells (FIG. 7).

We have previously observed that agonists that activate the 5-HT2A receptor, including serotonin, enhance cellular replication patterns. During 5-HT2A receptor KD cell line isolation, we observed that the cells that exhibited the greatest knock down of 5-HT2A receptor expression also did not replicate as quickly as matched WT ARPE-19 cells. We therefore assessed the replication kinetics of KD cells relative to the WT control cells.

CONCLUSION: Replication of all individual isolates of ARPE-19 cells that were knocked down for 5-HT2A receptor expression was suppressed. Of the ARPE-19 KD isolates, ARPE-SORTC4, which expressed the most 5-HT2A, had the growth rate, but its replication was still significantly suppressed relative to matched ARPE-19 WT controls. These findings supported why we did not isolate complete 5-HT2A Receptor knock out cells, since it did appear that 5-HT2A receptor expression imparts a growth/survivability advantage to these cells.

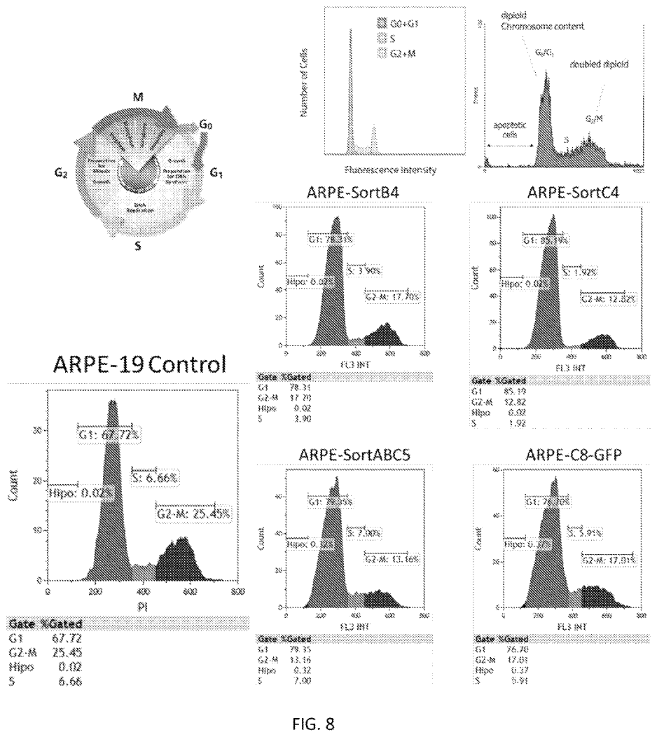

5-HT2A Receptor KD Cells Exhibit Disruption of Cell Cycle Phase Distribution Consistent with Cell Senescence (FIG. 8).

Shown to the right is a typical cell cycle distribution pattern from FACS that defines the stages gates of cells and stages of cell cycle associated with each region. Cells were fixed in 70% ethanol and assessed for cell cycle distribution using CycleFx PI/RNAse staining solution. Consistent with the decreased rates of cellular replication of ARPE KDs compared to WT controls, initial preliminary assessments of ARPE KD cells indicated that a higher proportion of cells were within G1. By comparison, we have previously observed that 5HT2A agonists, including serotonin, enhance replication of cells. Therefore, the decreased replication of 2A knockdowns is consistent with these findings.

CONCLUSION: A G0-G1 or G2-M growth arrest can be indicative of cells that are pre-senescent or have exited the cell cycle into cellular senescence—a major contributor to diseases associated with aging, including in age-related diseases of the eye. Initial findings on cell cycle analysis are consistent with the possibility that 5-HT2A receptor KD cells can have a pre-senescent or senescent phenotype that is reminiscent retinal epithelia in aged eyes.

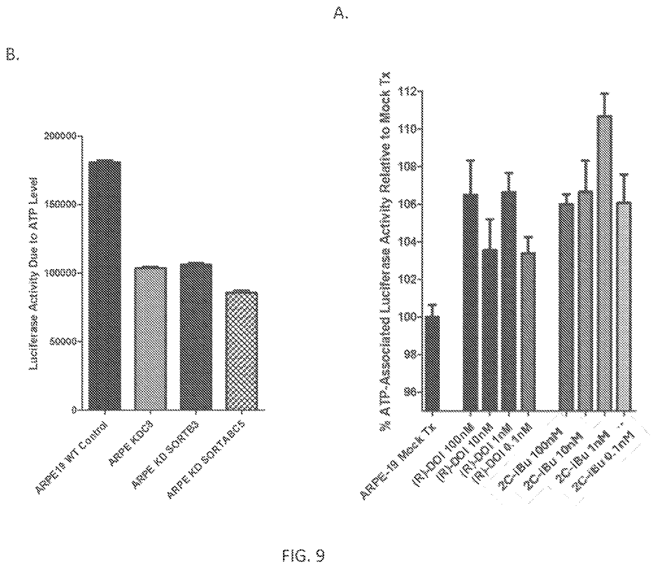

ATP Production is Decreased in 5-HT2A Receptor KD Cells Consistent with Mitochondrial Dysfunction, Whereas by Contrast, 5HT2A Agonists Enhance ATP Production and Mitochondrial Membrane Potential in WT ARPE-19 Cells (FIG. 9).

Consistent with previous data that we have provided on 5HT2A receptor-associated mitochondrial function, 5HT2A KD cells had lower levels of ATP detectable compared to ARPE WT controls. This is consistent with the contributions of certain components of the cytoskeleton and junctional complexes regulating mitochondrial dysfunction. Furthermore, in agreement with previously provided data on mitochondrial functional changes, 5HT2A agonists, R-DOI and 2C-iBu enhanced production of ATP on ARPEWT cells 24 h after administration.

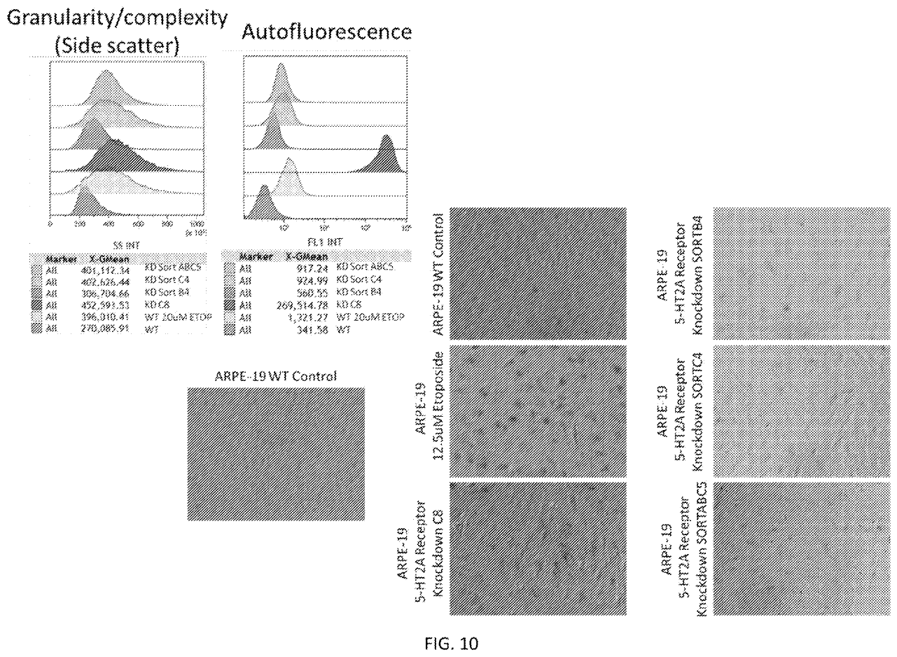

5-HT2A Receptor Knockdown Cells Exhibit Increased Granularity, Lipofuscin-Associated Autofluorescence, as Well as Frequency and Levels of Expression of SA-B-Galactosidase, which is Consistent with a Pre-Senescent/Senescent Cell Phenotype and Age-Related Diseases of the Retina (FIG. 10).

FACS analysis of 5HT2A KD cells indicated that not only did these cells increase in size, but also exhibited an increase in overall granularity and autofluorescence. Increased granularity is present within many diseases of aging and is a hallmark of progression towards cell senescence. It is believed that increased granularity is due to changes in the ability of the cells to properly process proteins through lysosomal or protein degradation pathways. These changes contribute to progression of many diseases of aging, including AMD, Alzheimer's and Parkinson's.

As cells are unable to properly process and degrade proteins, toxic structures begin to build up within the cell that induces disease associated pathologies, including lipofuscin within lysosomal complexes. Increases in cell autofluorescence is indicative of these changes and consistent with an aging phenotype, where lipofuscin build up is an indicator aging. As noted above, granularity and autofluorescence was increased in all KD cell lines. Although C8 exhibits high fluorescence, ARPE-19 KD C8 cells express GFP and therefore, the levels of fluorescence in the FL1 channel here are only indicative of GFP expression. Because of the presence of GFP, autofluorescence of this cell line can not be determined.

All indications were that all cell isolates knocked down for 5HT2A receptor expression exhibited hallmarks of progressing towards a cell senescent phenotype. To ascertain if these cell lines were in a state of pre-senescence, we assessed expression of functional SA-b-galactosidase, a standard marker of senescence. As expected, 5HT2A KD cells exhibited enhanced expression of b-gal, as well as an increased number of cells positive for its expression compared to matched ARPE-19 WT controls. As noted earlier, etoposide treated cells served as a positive control.

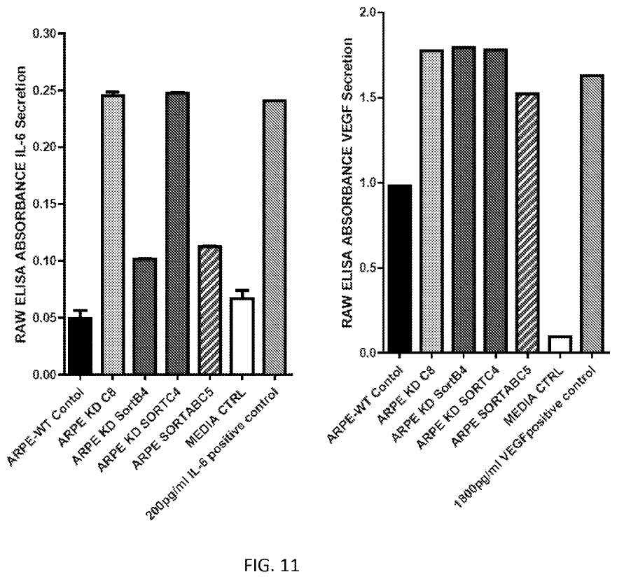

5-HT2A Receptor Knockdown Cells Exhibit Increased Expression of Inflammatory Cytokines that Contribute to Chronic Disease Progression, Numerous Retinopathies and AMD Development (FIG. 11).

The development of cell senescence is a hallmark of most diseases of aging and contributes to development chronic inflammation. Furthermore, it is a self-perpetuating process, as senescent cells enhance the probability of other cells converting to a senescent phenotype through its increased production of inflammatory cytokines. As noted below, preliminary ELISA assays of cell supernatants isolated from ARPE 5HT2A KD cells showed an increased presence of IL-6, IL-1 and VEGF compared to ARPE-19 WT controls. Given the cost of these assessments, we did not run full cytokine standard curves for these initial assessments, but did run a known standard positive control. Data is therefore reported as RAW absorbance values. It was noted for VEGF that as reported in the literature WT controls did express VEGF; however, its expression was enhanced in 5HT2KD cells. Furthermore, we noted that the absorbance values for most 5HT2A KD cell lines reached the saturation maximum; therefore, the relative levels here are likely under-reported. For Il-6 values, cell numbers of 5HT2A KD cells, SORTB4 and SORT ABC5 were 3 fold less than all other cells. This has influenced or been responsible for the reduced abs value for these cells compared to their other KD cell counterparts. These preliminary outcomes will be optimized and reproduced in the future. However, what is clear is that the development of these phenotypes in 5HT2A KD RPE enhance production and secretion of cytokines associated chronic inflammation and diseases of aging. Indeed, VEGF expression is a major contributing factor to wet AMD development and a major target of biologics for its treatment. These findings are also consistent with previous findings that we provided that tie 5HT2A receptor targeting with preventing vascular associated pathologies.

### Example 5

### Non-Limiting Exemplary Embodiments of the Disclosure

Without wishing to be bound by theory, the compounds described herein can comprise DOI (2,5-Dimethoxy-4-iodoamphetamine), R-DOI (R enantiomer 2,5-Dimethoxy-4-iodoamphetamine), DOC (2,5-Dimethoxy-4-chloroamphetamine), DOB (2,5-Dimethoxy-4-bromoamphetamine), DOF (2,5-Dimethoxy-4-fluoroamphetamine), DOET (2,5-Dimethoxy-4-ethylamphetamine), DOM (2,5-Dimethoxy-4-methylamphetamine), DOTFM (2,5-Dimethoxy-4-trifluoromethylamphetamine), 2C-isobutyl (2,5-Dimethoxy-4-isobutylphenethylamine), 2C-neopentyl (2,5-Dimethoxy-4-neopentylphenethylamine), 2C-B (4-bromo-2,5-dimethoxyphenethylamine) CAS #: 66142-81-2, 2C-C (2-(4-Chloro-2,5-dimethoxyphenyl)ethan-1-amine) CAS #: 88441-14-9, 2C-I (2-(4-Iodo-2,5-dimethoxyphenyl)ethan-1-amine) CAS #: 69587-11-7, 2C-N (2,5-dimethoxy-4-nitrophenethylamine) CAS #: 261789-00-8, and 2C-Bu (2,5-Dimethoxy-4-butylphenethylamine).

### Non-Limiting Highlights of the Work Described Herein Comprise:

The role of 5-HT2A receptor suppression in promotion of aging-associated disease processes, including lysosomal dysfunction, protein folding/processing disorders, inflammation, oxidative stress and cell senescence.

The ability of 5-HT2A receptor activation to prevent aging-associated disease processes.

The therapeutic efficacy of 5-HT2A receptor agonists in the phenylethylamine class to prevent aging-associated cell senescence and senescence promoting disease processes.

The low therapeutic levels of an exemplary phenylethylamine 5-HT2A receptor agonist in preventing aging-associated cell senescence and senescence promoting processes.

The specific dosing/target concentrations of 5-HT2A agonists required to prevent and resolve aging-associated disease processes and senescence.

The identification of a specific patient population that can be responsive to treatment at these low therapeutic doses, inclusive of any condition of hyperglycemia or underlying diabetic complications.

The required dosing concentrations required of these agonists to prevent high glucose-mediated aging-associated disease processes and development of cell senescence.

The ability of 5-HT2A receptor agonists to prevent and resolve aging-associated disease processes within retinal cells, including retinal pigmented epithelia, at the back of the eye.

The ability of 5-HT2A receptor agonists to prevent and resolve disease associated with aging of retinal cells including diabetic retinopathies and age-related macular degeneration.

Protection of neural-associated cells from hyperglycemic effects in diseases such as diabetic neuropathy.

Prevention or treatment of lysosomal and peroxisomal function diseases, including, but not limited to Alzheimer's disease, Parkinson's, and Gaucher's disease.

Prevention or treatment of diseases of metabolic function.

Prevention or treatment of neurodegenerative diseases with underlying pathogenesis related to metabolic dysfunction, lysosomal dysfunction, cytoskeletal abnormalities, ROS or cell senescence including, but not limited to, Alzheimer's, Parkinson's, and Huntington's diseases.

Prevention or treatment of diseases characterized by cytoskeletal abnormalities including, but not limited to Alzheimer's disease, Parkinson's disease, cerebral ischemia, multiple sclerosis, and amyotrophic lateral sclerosis (stress fiber associated diseases: atherosclerosis, osteoporosis, etc)

Prevention or treatment of proliferative diseases including, but not limited to cancer, atherosclerosis, rheumatoid arthritis, psoriasis, idiopathic pulmonary fibrosis, scleroderma, and cirrhosis of the liver.

### Allows for Identification of Target Patient Population:

Patients with Hyperglycemia
Drug induced hyperglycemia
Pathogen-induced hyperglycemia
Diabetic Mellitus
Type 1 diabetes
Type 2 diabetes
Gestational Diabetes
Patients with Pre-Diabetes
Insulin resistant patients
Insulin-resistance due to Cushing's, other endocrine disorders
Stress-Induced hyperglycemia
Patients with Increased Circulating or tissue localized reactive oxygen species
Aging patients Pancreatic disorders inducing hyperglycemia, such as chronic pancreatis, pancreatic
cancers, cystic fibrosis, etc.
Patients with metabolic dysfunction
Patients with Neurodegenerative-associated Diseases
Enables treatment or prevention of causes of disease indications such as:
Diabetic retinopathies
Diabetic neuropathy
Diabetic macular edema
Cataracts
Glaucoma
Age-related macular degeneration of RPE and retinal ganglionic cells
Non-diabetic associated neuropathies
Diseases of lysosomal dysfunction
Diseases of metabolic dysfunction
Diseases of proliferative dysfunction
Diseases of cytoskeletal dysfunction
Neurodegenerative Diseases

In embodiments, patient population treatment targets can be identified. For example, mitochondrial dysfunction, which is visualized by changes in retinal metabolism, can be measured in the clinic via increased intensity of autofluorescent flavoprotein in the retina.

Without wishing to be bound by theory, the following non-limiting, exemplary patient populations will be responsive to treatment:
At the transition age of ocular disease development 50 to 60 years of age prior to 5-HT2A receptor decline and increased dosing levels at greater than 60 years of age.
Prediabetic patients with blood glucose levels between 6-6.9 mmol/L with most effective outcomes in diabetic patients that have blood glucose levels >7 mmol/L.
Patients from 50 to 60 years of age that present with early aberrant lipofuscin and drusen depositions on retinal exam.
Patients from 50 years and older that present with autofluorescent lipofuscin and drusen deposition on retinal exam.
Patients 50 years and older that are pre-diabetic or diabetic as defined above, with autofluorescent lipofuscin or drusen deposits that have begun to exhibit alterations in RPE structure.
Diabetic patients 50 years or older that have begun to experience blurry and distorted central vision with the presence of autofluorescent lipofuscin or drusen deposits.
The target AMD population where activity can be targeted are patients clinically defined as having Early AMD (small to medium drusen, with AMD pigmentary abnormalities and impaired dark adaptations) to Intermediate AMD (one large druse, with AMD pigmentary abnormalities and impaired dark adaptations).
Diabetic patients with an indication of retinopathies via assessment on the Early Treatment Diabetic Retinopathy Study (ETDRS) disease severity score and/or presence of retinopathy via macular OCT.

Mitochondrial dysfunction can be measured in the eye via methods known in the art. See for example, Chen et al. Functional imaging of mitochondria in retinal diseases using flavoprotein fluorescence. Eye (Lond). 2021 January; 35(1):74-92. The presence of autofluorescence oxidized flavoproteins in the retina as observed by Fundus microscopy with fluorescence in the 465 nM band range. Changes in ocular retinal metabolism as observed by clinical imaging using metabolic detectors on a retinal metabolic imaging device, that are optimized for detection of autofluorescent oxidized flavoprotein. Treatment with drug can be indicated for patients exhibiting values of fluorescent flavoproteins intensity greater than normal values of their respective age group.

### Non-Limiting Routes of Administration:

For Age-Related Diseases of the Eye: (1) Topical eye drop. PK and PD data has shown that a topically applied phenethylamine can penetrate the eye and make it to the back of the eye at concentrations consistent with the sustained levels required to achieve the effects described herein. Therefore, a topical application in normal FDA acceptable inert excipients can be utilized. This can include compounds that increase resonance time such as carboxymethylcellulose (CMC), polyethylene glycol (PEG), or HPCM. For example, we have utilized formulations with CMC at 0.5-2% with low to high viscosities with success. Additional inclusion of compounds like polysorbate 80, Tween, etc. can increase both penetration and compound solubility. In embodiments, the solution can be isotonic, and in the pH range of 5 to 7.5. A basic solution for ocular delivery can cause alkaline burns to the cornea. Non-limiting advantages to this route are less potential for CNS exposure and behavioral liabilities. (2) Impregnated slow-release contact lens or ocular hydrogel. For chronic disease indications, this is a very good method to not require multiple topical applications. (3) For sustained continuous delivery, the compound could be impregnated into a slow-release biodegradable or non-biodegradable polymer or device and implanted into the eye as either an intravitreal or subconjunctival implant. These intraocular implants have advantages in less potential for CNS exposure and behavioral liabilities. Sustained delivery levels can match PK/PD data and to achieve concentrations consistent with those identified in the data to be required to elicit responses shown in datasets. Those are generally in the low nanomolar ranges. This is a big area for these types of drug delivery and gets over some of the potential DEA diversion issues with these compounds. (4) Direct intravitreal injection into the eye. Advantages to this route comprise less potential for CNS exposure and behavioral liabilities. (5) Systemic delivery via oral or IV. Radioactive 5HT2A agonists delivered systemically can concentrate within retinal tissues to levels that are within the range to achieve and affect outcomes.

For Age-Related Diseases of the CNS: (1) Systemic delivery via oral or IV. These drugs have an inherent ability to penetrate into the CNS and reach target neurons that are sensitive to neurodegenerative diseases. The processes of neurodegeneration, like Alzheimer's and Parkinson's are like what is shown in RPE, which show signs of disease decades before presentation of neurodegenerative disorders developing in brain.

### Example 6

### The Role of the Serotonin 2A Receptor (5-HT2AR) in Retinal Pigmented Epithelial Cell-Associated Development and Progression of Age-Related Macular Degeneration

Dysregulation of aging cells leads to chronic inflammation and pathogenesis (FIG. 12).
Age-related diseases are becoming more prevalent in our population
Modem medicine extends life-span by preventing or slowing life-threatening diseases.
Even if we combat life-threatening pathogen, our cells will succumb to aging.
Over time, cellular aging causes chronic inflammation which promotes the development of cellular senescence and age-related diseases [15].
An understanding cellular aging mechanisms will provide insight to targets which can prevent/treat age-related diseases.
Age-related diseases can affect a plethora of system for example, retinopathy, neurodegenerative disorders, diabetes, cancer, heart disease, and more. While there are common hallmarks of aging, the molecular signatures can be unique to the cell type.
Metabolic Dysfunction in Diabetes is a risk factor for several age-related diseases:
The diabetic condition is characterized by glucose intolerance and insulin resistance.
Over time, metabolic dysregulation in diabetes increases ROS and causes chronic inflammation leading to age-related diseases [23].
Predispositions include retinopathy, neurodegeneration, cardiovascular disease, and nephropathy [1,23].
Age-related macular degeneration (AMD) is characterized by disruption of the retinal pigmented epithelium (RPE) (FIG. 13).
Age-related macular degeneration (AMD) is the leading cause of blindness in elderly.
AMD progression is characterized by impaired metabolism, extracellular drusen deposition(lipids and protein), increased inflammation and ROS, vascularization, and the accumulation of senescent cells
The degeneration of the retinal pigmented epithelium (RPE) is observed in AMD in the clinical setting

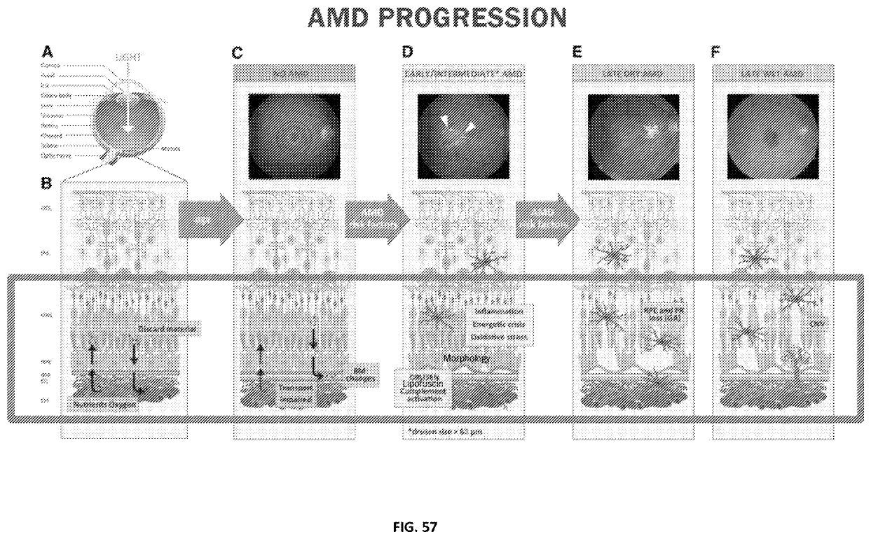

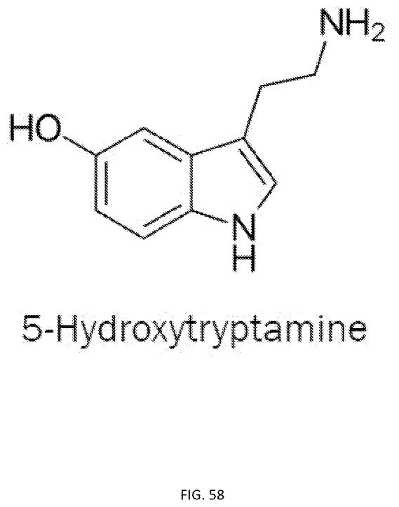

### AMD Progression (FIG. 57)

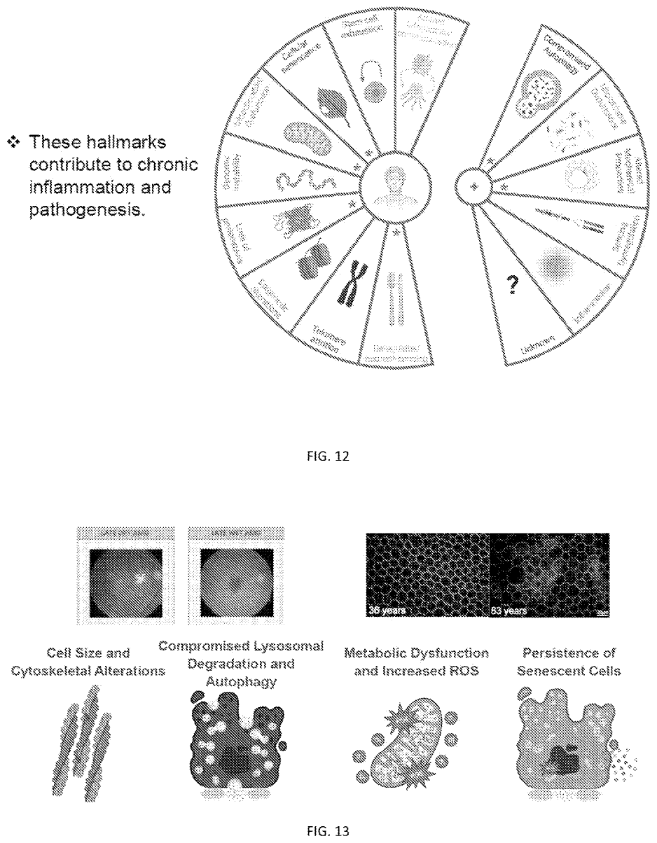

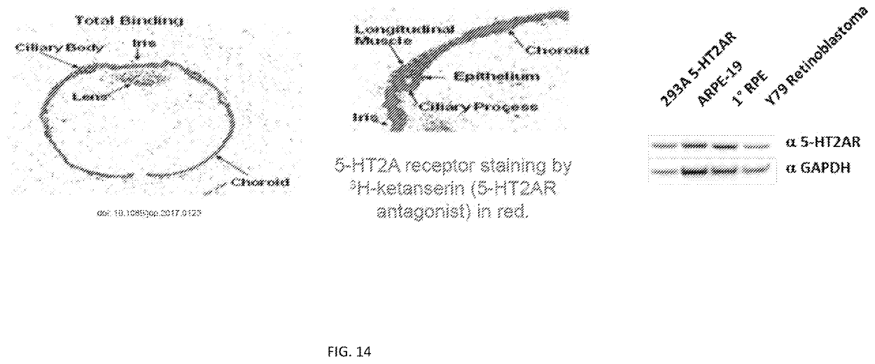

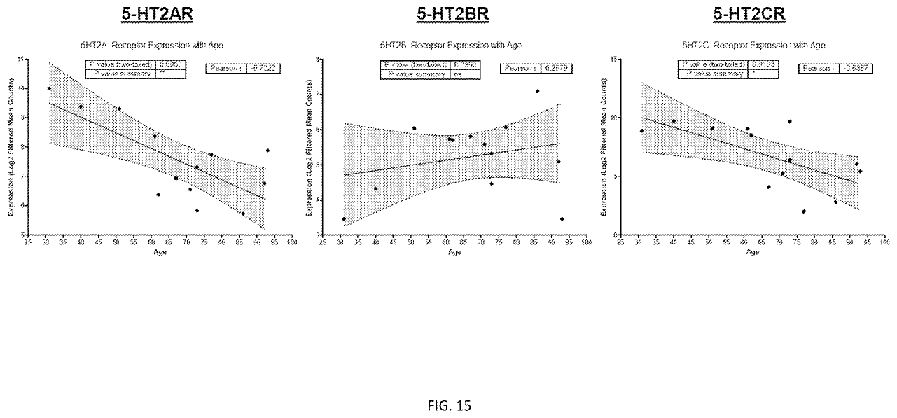

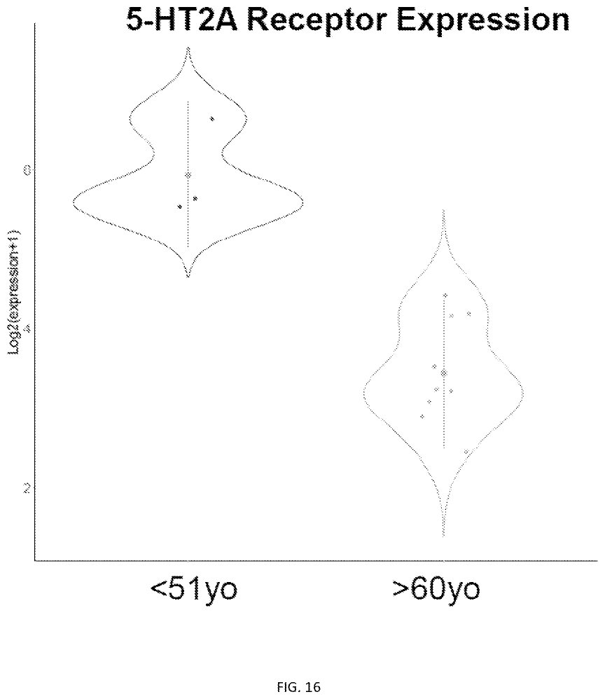

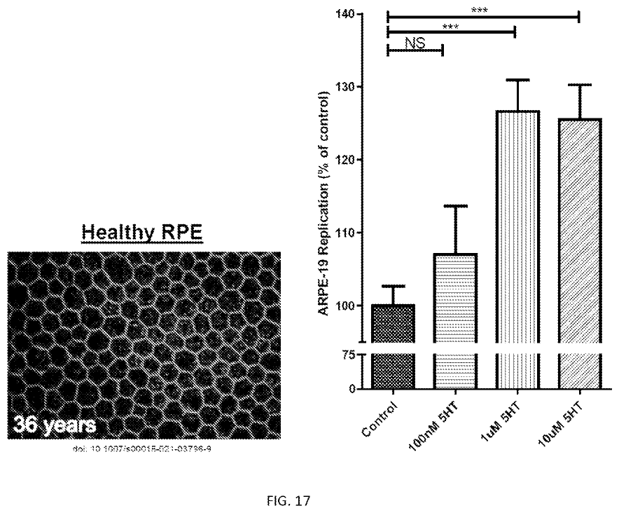

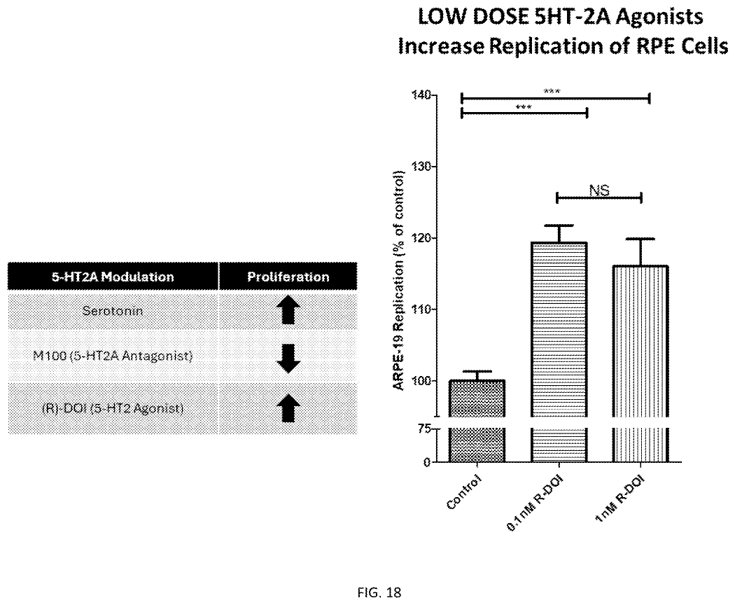

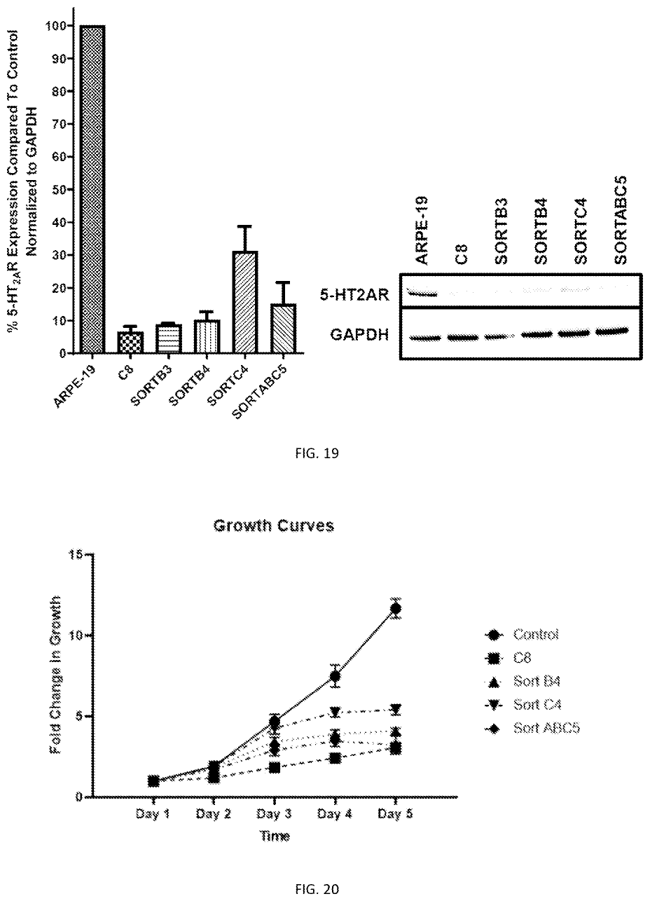

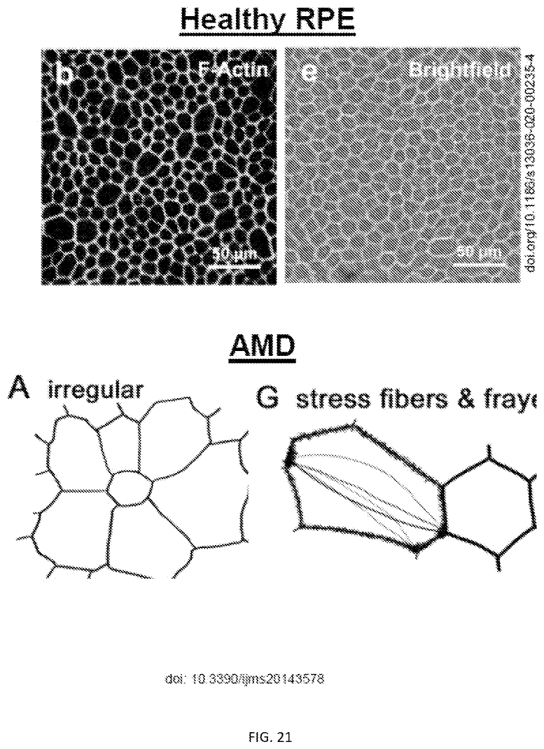

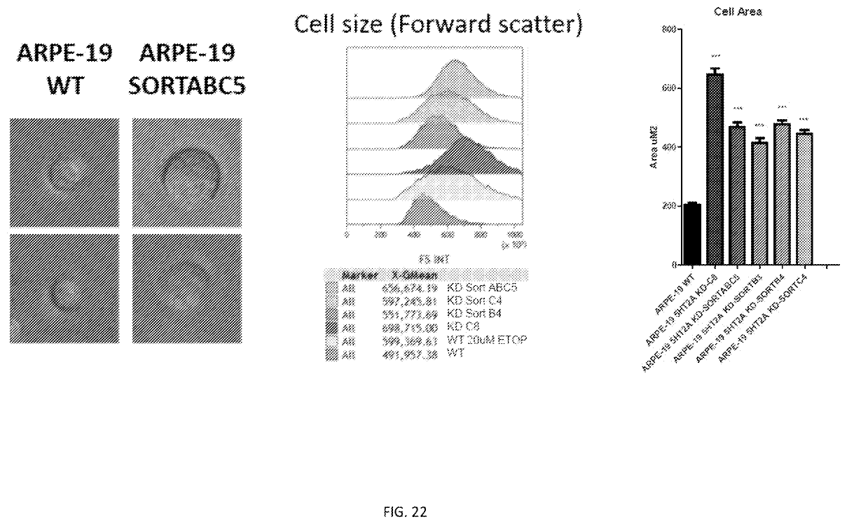

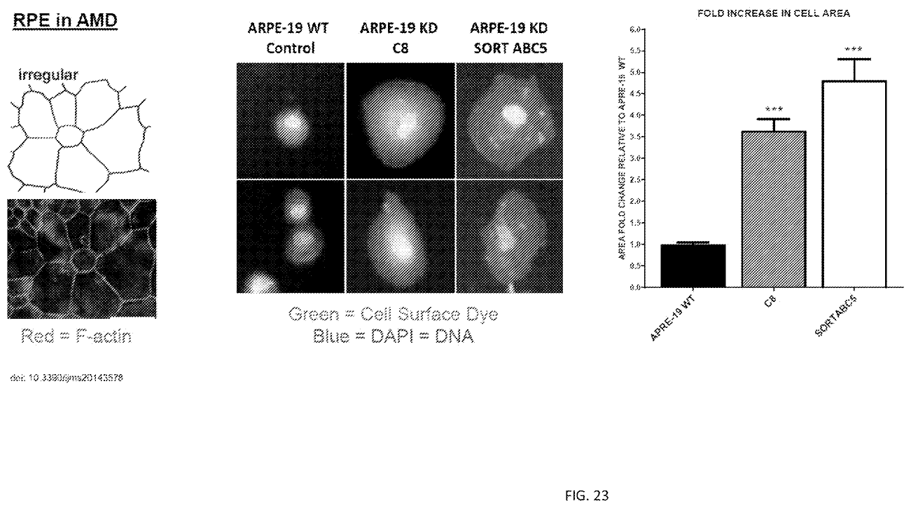

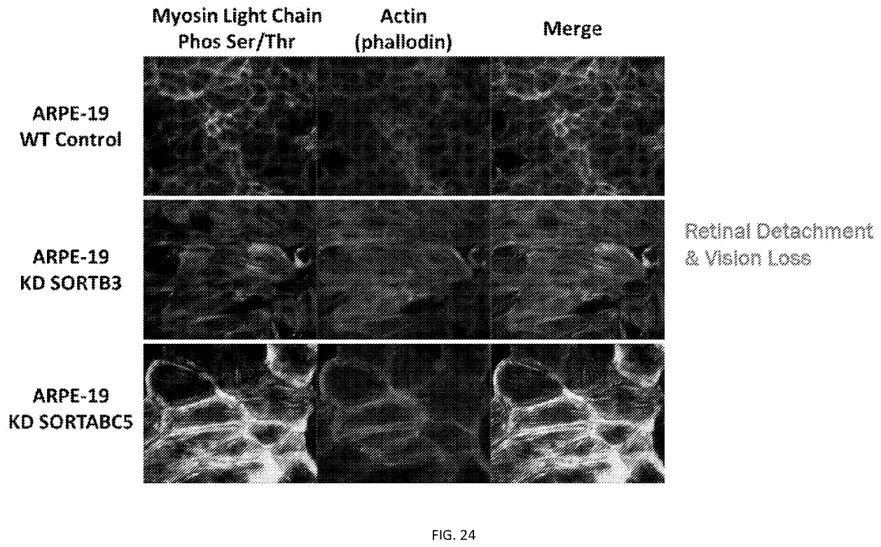

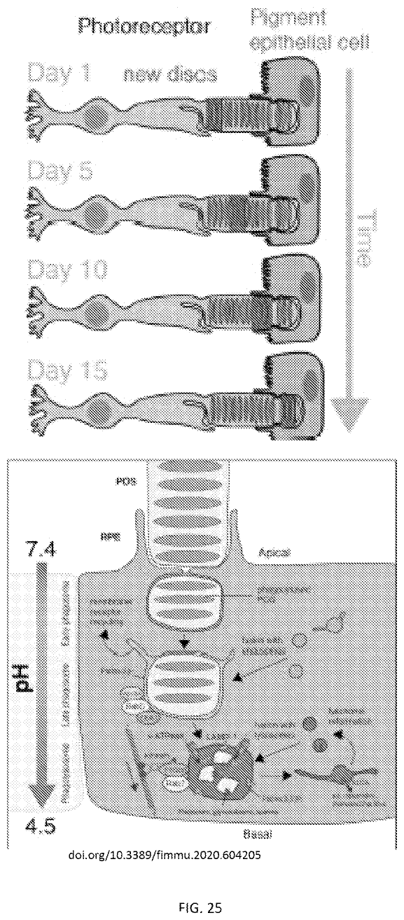

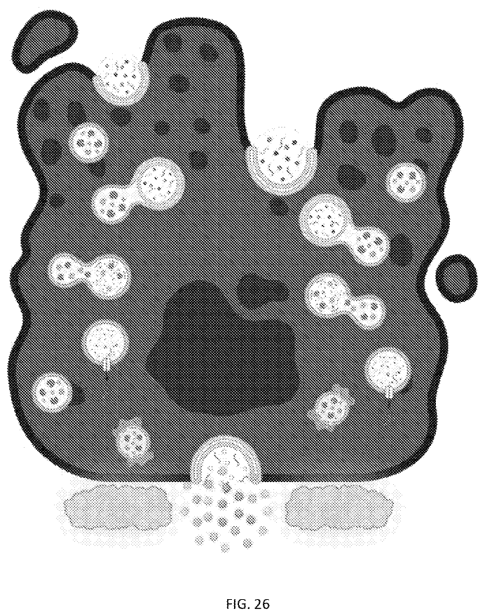

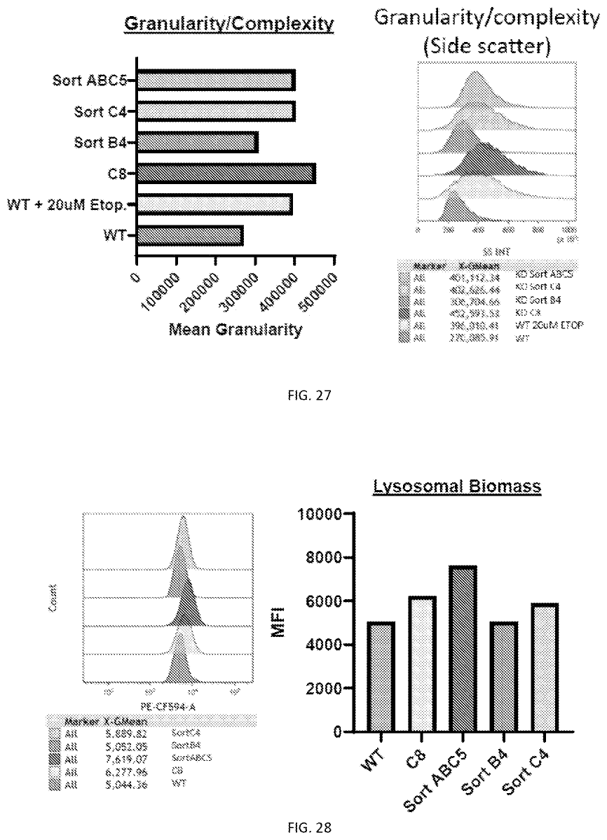

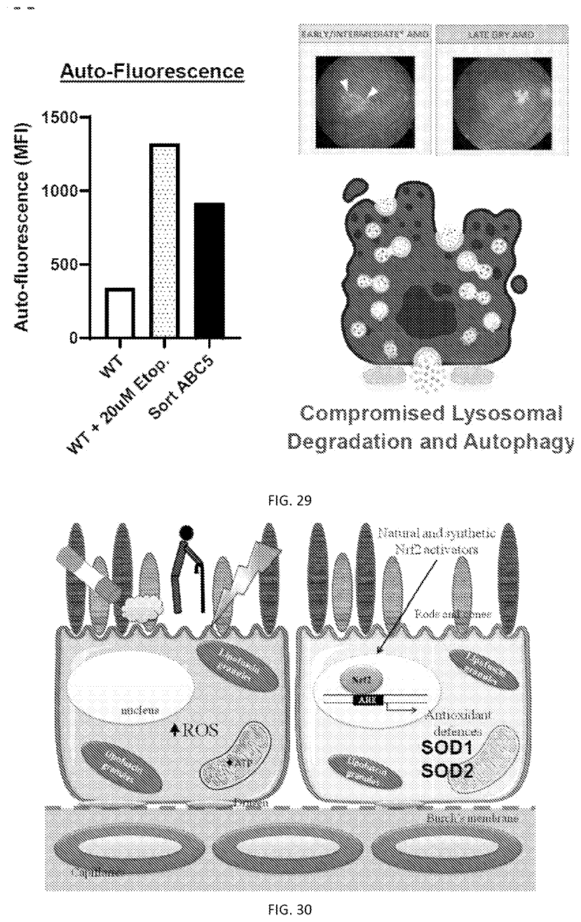

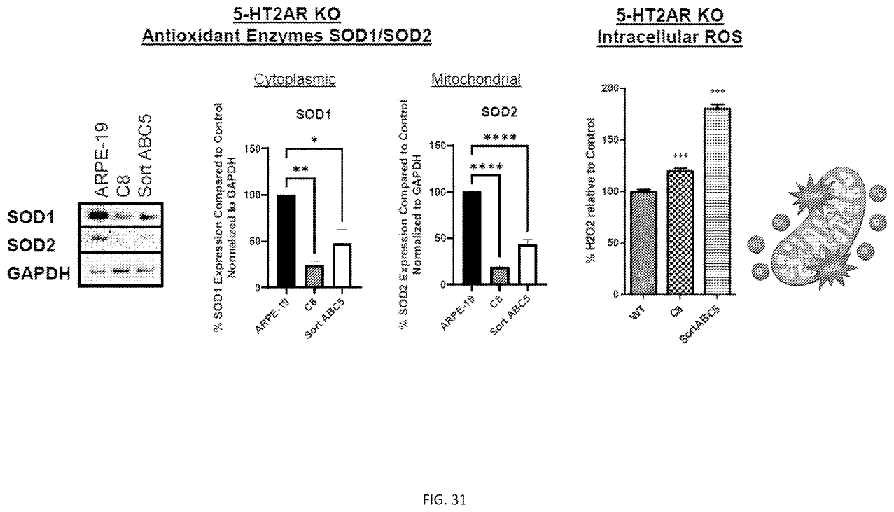

Functions of Serotonin: essential for retinal development and health, regulates metabolism, improves insulin sensitivity, and modulates inflammation and the vasculature.
Unfortunately, the serotonergic system declines with age. Additionally, the decline of serotonin receptors and transporters has been documented in the aging nervous system. In the nervous system, the decline of the serotonin 2A (5-HT2A) receptor is associated with age-related diseases, like in Alzheimer's and Parkinson's disease (FIG. 58)
The 5-HT2A receptor is highly localized and expressed within the vertebrate eye, especially within the retinal pigmented epithelium (RPE). The localization and expression can indicate functional roles in vision (FIG. 14).
Retinal 5-HT2A and 5-HT2C Receptor Expression Decreases with Age
Retrospective analysis of RNAseq data from clinically derived RPE (FIG. 15).
The decline of the 5-HT2A receptor coincides with the hot-spot for retinopathy development. Retinopathy frequently develops during the 50- to 60-year-old transition (FIG. 16).
Thus, without wishing to be bound by theory, age-related repression of 5-HT2AR contributes to disease processes associated with AMD progression.
The role of serotonin and 5-HT2AR in RPE Cellular Replication:
Physiologically relevant 5-HT levels increase cellular replication in health RPE cells. This increase in proliferation promotes the formation of a compact RPE-monolayer which is important for promoting a healthy blood-retinal barrier (FIG. 17).
The 5-HT2A Receptor is a major driver of serotonin-induced cellular proliferation (FIG. 18).
To facilitate assessment of the role of 5-HT2AR in age-related diseases of the eye, we generated 5-HT2AR knockouts in ARPE-19 cells. We generated 5-HT2AR knockouts via CRISPR/Cas9 in adult retinal pigmented epithelial cells from a healthy 19-year-old male (ARPE-19) (FIG. 19).
Consistent with our pharmacological analysis, 5-HT2A receptor knockdowns exhibit decreased proliferation (FIG. 20).
Morphology: 5-HT2A receptor knockdown increases cell size and affects cytoskeletal structure.
RPE size, morphology, and cytoskeletal network are altered during AMD. Healthy RPE are a single layer of highly-organized, polygonal cells with similar size and shape, maintaining a healthy blood-retinal barrier. Cytoskeletal elements influence cell size, morphology, subcellular trafficking, and multi-cellular communication. The cytoskeletal network in the RPE is important for maintaining a healthy state of elasticity and permeability. Cytoskeletal dysregulation impacts RPE integrity and is a well-documented feature of retinopathy, including AMD. (FIG. 21)
5-HT2A Receptor knockdowns are larger than the ARPRE-19 WT. We noticed the 5-HT2A receptor knockdowns were larger than the ARPE-19 WT (FIG. 22).
5-HT2AR knockouts exhibit increased cellular area consistent with dysfunctional RPE in AMD (FIG. 23).
Partially explaining morphological differences, 5-HT2A knockouts exhibit stress fiber cytoskeletal structures consistent with retinopathy. Colocalization of P-MLC with F-actin induces cellular contraction. (FIG. 24)
Function: 5-HT2A Receptor Knockdowns Exhibit Lysosomal Dysfunction
Cytoskeletal rearrangement affects phagocytosis and intracellular trafficking of cargo destined for degradation (FIG. 25). One major function of the RPE is to phagocytose cellular debris and photoreceptor out segments in the retina and subject it to lysosomal degradation. Dysregulation of the autophagy-lysosomal system is a well-described characteristic of neurodegenerative disorders, including AMD.
Inability to efficiently degrade these protein/lipids results in buildup of auto-fluorescent deposits in the RPE, termed drusen bodies (FIG. 26).
5-HT2A Receptor knockdowns are more granular than the ARPE-19 WT (FIG. 27). Increased cell granularity is indicative of lysosomal dysfunction, protein aggregation, and vacuolization.
Indicative of lysosomal dysfunction, 5-HT2A receptor knockdowns have increased lysosomal biomass compared to the ARPE-19 WT (FIG. 28). Lysosomal dysfunction is recognized as an important factor for age-related and neurodegenerative diseases.
5-HT2A Receptor knockdowns have an auto-fluorescent phenotype consistent with lipofuscin accumulation (FIG. 29). Lipofuscin is a complex of lipids, protein, and co-factors that auto-fluoresce. Lipofuscin is a complex of lipids, protein, and co-factors that auto-fluoresce. Lipofuscin accumulation facilitates drusen deposition, a major characteristic of AMD.
Lysosomal dysfunction is associated with metabolic dysregulation and increased oxidative stress (FIG. 30). Metabolic dysregulation is a common feature of diabetic retinopathies, causing increased levels of ROS. This increased ROS is accompanied by decreased expression of cellular antioxidants. Increased ROS levels are a known cause of cellular damage leading to the manifestation of age-related and neurodegenerative diseases, like AMD.
5-HT2A KOs exhibit decreased expression of cytoplasmic/mitochondrial antioxidants and increased levels of ROS (FIG. 31).

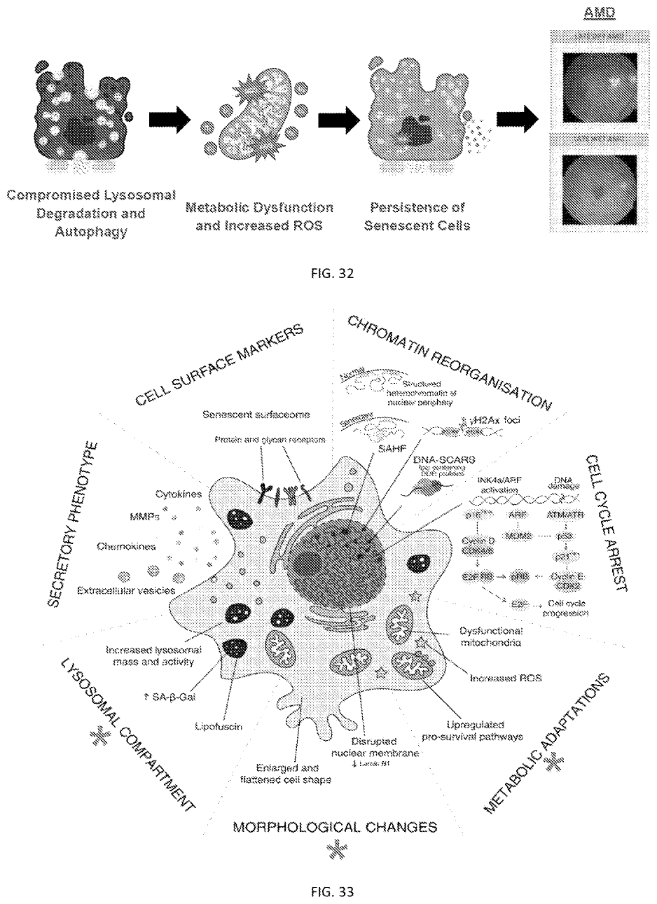

Cellular Senescence: 5-HT2A Receptor Repression Induces a Senescent-Like Phenotype
ROS is a driver of cellular senescence in AMD and other age-related diseases (FIG. 32).

What is cellular senescence? Senescence is the cellular equivalent to aging and is defined as ‘irreversible’ cell-cycle arrest.

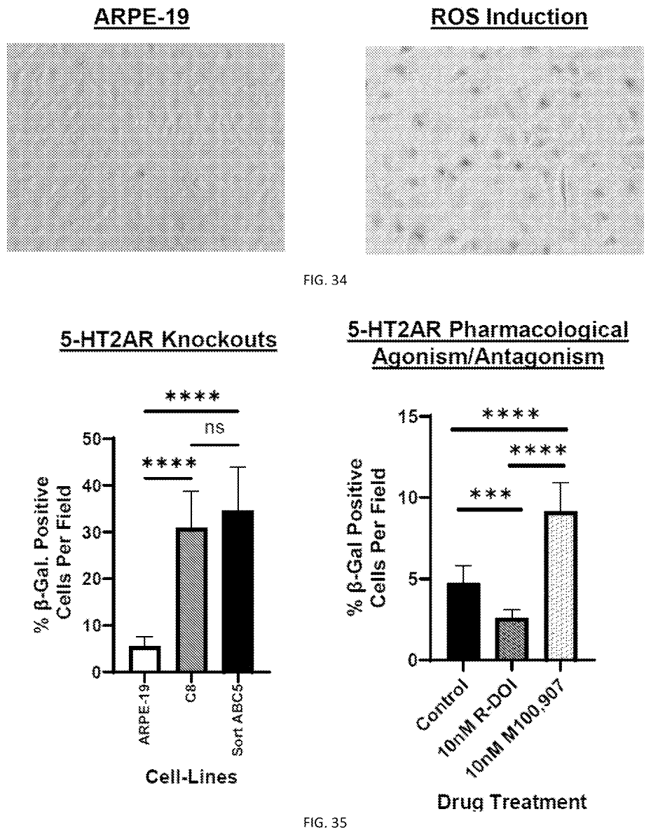

Senescent cell characteristics include limited proliferation, enlarged cell size, excessive ROS, metabolic and proteolytic dysfunction, senescence-associated secretory phenotype (pro-inflammatory cytokines), nutrient imbalances, telomere attrition, DNA damage, and epigenetic dysregulation. In addition to these characteristics, senescence-associated β-galactosidase(SA-β-Gal) staining is the gold standard for quantifying cellular senescence.
Hallmarks of Senescence (FIG. 33)
ARPE-19 and ROS induction (FIG. 34)
5-HT2A Knockout and pharmacological antagonism induces a senescent-like phenotype (FIG. 35)

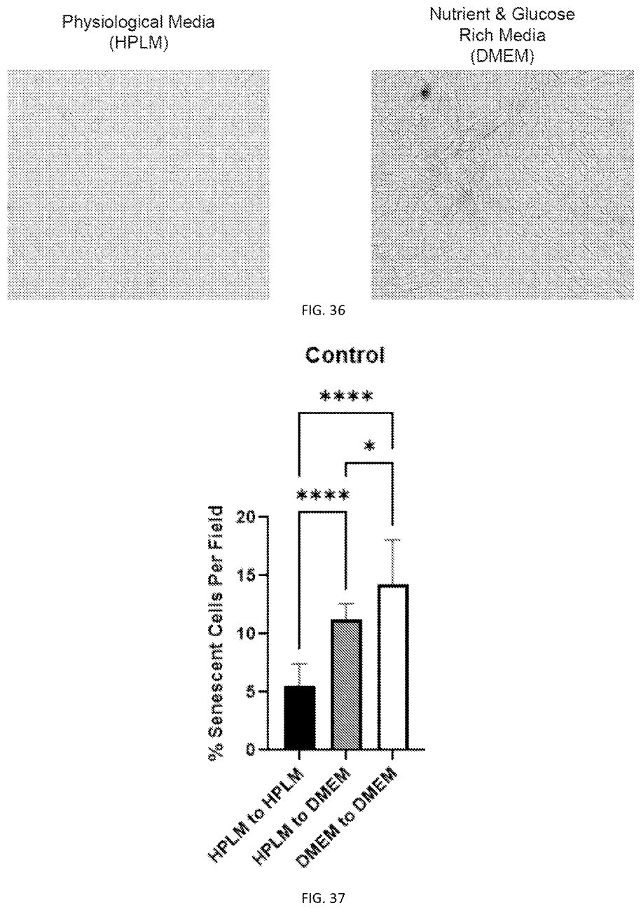

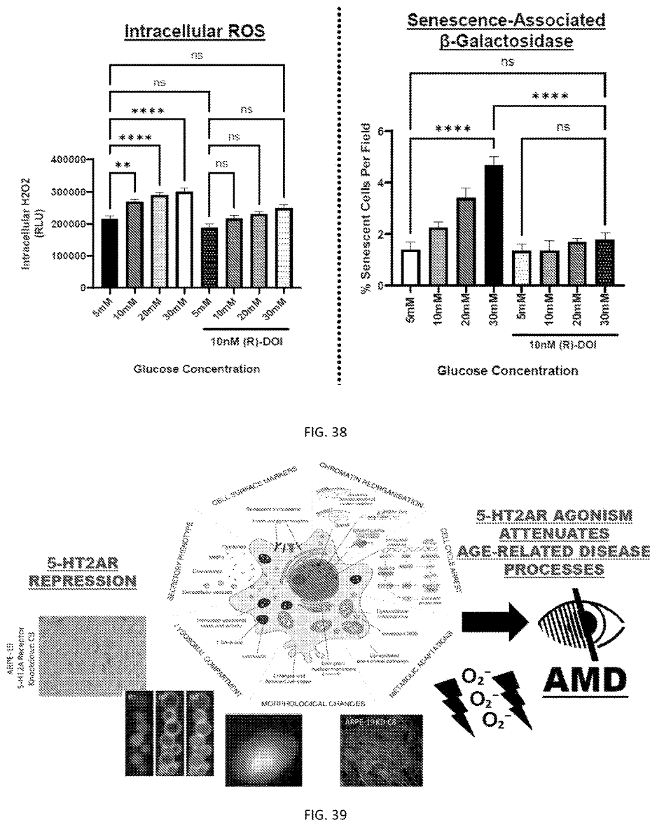

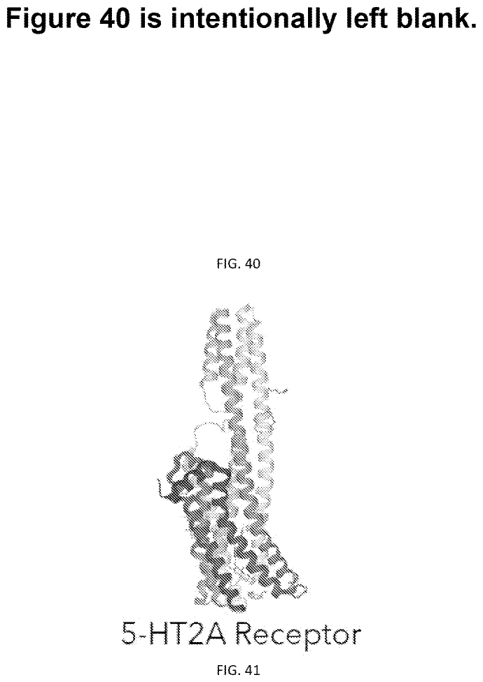

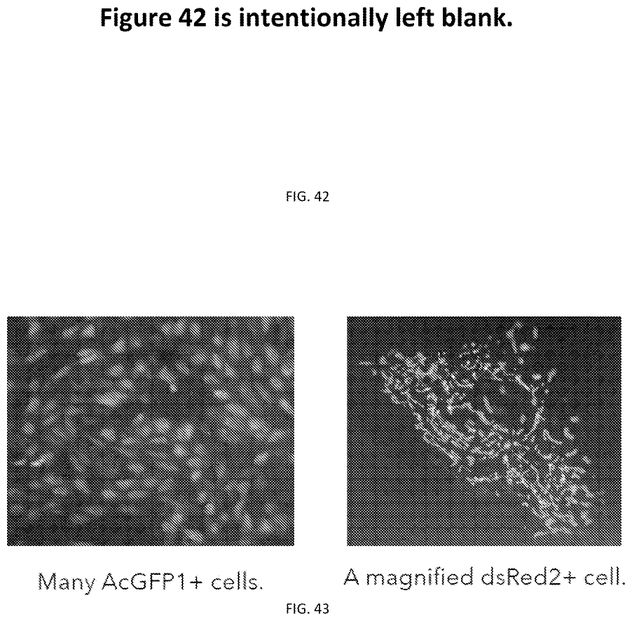

Diabetic Retinopathy: 5-HT2A Receptor Agonism Alleviates Glucose-Induced ROS and Cellular Senescence. Metabolic disturbances in diabetes are a risk factor for AMD and other retinopathies. Diabetes (glucose intolerance and insulin resistance) generates increased ROS and cellular senescence which causes AMD and retinopathies.
Nutrient rich conditions increase levels of senescence-associated beta-galactosidase in ARPE-19 (FIG. 36).
The ARPE-19 Senescent-like phenotype is reduced in physiological media compared to DMEM/F-12 (FIG. 37). Human plasma-like media (HPLM) is a media comparable to physiological conditions. DMEM/F-12 is a nutrient rich media which has increased glucose, pyruvate, and other nutrients.
5-HT2AR Agonism alleviates glucose-induced ROS and cell senescence (FIG. 38).
Non-Limiting summation (FIG. 39)

### REFERENCES CITED IN THIS EXAMPLE

[1] Gregg E W, Engelgau M M, Narayan V. Complications of diabetes in elderly people. BMJ. 2002 Oct. 26; 325(7370):916-7. doi: 10.1136/bmj.325.7370.916. PMID: 12399324; PMCID: PMC1124430.
[2] Armento A, Ueffing M, Clark S J. The complement system in age-related macular degeneration. Cell Mol Life Sci. 2021 May; 78(10):4487-4505. doi: 10.1007/s00018-021-03796-9. Epub2021 Mar. 9. PMID: 33751148: PMCID: PMC8195907.
[3] Azmitia E C. Serotonin neurons, neuroplasticity, and homeostasis of neural tissue. Neuropsychopharmacology. 1999 August:21(2 Suppl):33S-45S. doi: 10.1016/S0893-133X(99)00022-6. PMID: 10432487.
[4] Chen X, Rong S S, Xu Q, Tang F Y, Liu Y, Gu H, Tam P O, Chen L J, Brelén ME, Pang C P, Zhao C. Diabetes mellitus and risk of age-related macular degeneration: a systematic review and meta-analysis. PLoSOne. 2014 Sep. 19; 9(9):e108196. doi: 10.1371/journal.pone.0108196. PMID: 25238063; PMCID: PMC4169602.
[5] Gilbert S F. Developmental Biology. 6th edition. Sunderland (MA): Sinauer Associates; 2000. Aging: The Biology of Senescence.
[6] Karrer T M, McLaughlin C L. Guaglianone C P. Samanez-Larkin G R. Reduced serotonin receptors and transporters in normal aging adults: a meta-analysis of PET and SPECT imaging studies. NeurobiolAging. 2019 August:80:1-10. doi: 10.1016/j.neurobiolaging.2019.03.021. Epub2019 Apr. 5. PMID: 31055162: PMCID: PMC6679764.
[7] Lu T C. Brbic M, Park Y J, Jackson T, Chen J, Kolluru S S, Qi Y, Katheder N S, Cai X T, Lee S. Chen Y C, Auld N, Liang C Y, Ding S H, Welsch D, D'Souza S, Pisco A O, Jones R C, Leskovec J, Lai E C, Bellen H J, Luo L, Jasper H, Quake S R, Li H. Aging Fly Cell Atlas identifies exhaustive aging features at cellular resolution. Science. 2023 Jun. 16; 380(6650):eadg0934. doi: 10.1126/science.adg0934. Epub2023 Jun. 16. PMID: 37319212.
[8] Mazzoni F. Safa H, Finnemann S C. Understanding photoreceptor outer segment phagocytosis: use and utility of RPE cells in culture. Exp Eye Res. 2014 September; 126:51-60. doi: 10.1016/j.exer.2014.01.010. Epub2014 Apr. 26. PMID: 24780752; PMCID: PMC4145030.
[9] McHugh D, Gil J. Senescence and aging: Causes, consequences, and therapeutic avenues. J Cell Biol. 2018 Jan. 2:217(1):65-77. doi: 10.1083/jcb.201708092. Epub2017 Nov. 7. PMID: 29114066; PMCID: PMC5748990.
[10] Moiseiwitsch J R, Lauder J M. Serotonin regulates mouse cranial neural crest migration. Proc Natl AcadSci USA. 1995 Aug. 1; 92(16):7182-6. doi: 10.1073/pnas.92.16.7182. PMID: 7638165; PMCID: PMC41303.
[11] Moreno-Garcia A, Kun A, Calero O, Medina M, Calero M. An Overview of the Role of Lipofuscin in Age-Related Neurodegeneration. Front Neurosci. 2018 Jul. 5; 12:464. doi: 10.3389/fnins.2018.00464. PMID: 30026686; PMCID: PMC6041410.
[12] Morgan D G. The dopamine and serotonin systems during aging in human and rodent brain. A brief review. Prog NeuropsychopharmacolBiol Psychiatry. 1987; 11(2-3):153-7. doi: 10.1016/0278-5846(87)90053-4. PMID: 2819950.
[14] PootanakitK, Prior K J, Hunter D D, Brunken W J. 5-HT2a receptors in the rabbit retina: potential presynaptic modulators. Vis Neurosci. 1999 March-April:16(2):221-30. doi: 10.1017/s0952523899162035. PMID: 10367957.
[15] Sanada F, Taniyama Y, Muratsu J, Otsu R, Shimizu H, Rakugi H, Morishita R. Source of Chronic Inflammation in Aging. Front Cardiovasc Med. 2018 Feb. 22; 5:12. doi: 10.3389/fcvm.2018.00012. PMID: 29564335: PMCID: PMC5850851.
[16] Sharif N A. iDrugs and iDevicesDiscovery Research: Preclinical Assays. Techniques, and Animal Model Studies for Ocular Hypotensives and Neuroprotectants. J OculPharmacolTher. 2018 January/February; 34(1-2):7-39. doi: 10.1089/jop.2017.0125. Epub2018 Jan. 11. PMID: 29323613.
[17] Somasundaran S, Constable I J, Mellough C B, Carvalho L S. Retinal pigment epithelium and age-related macular degeneration: A review of major disease mechanisms. Clin Exp Ophthalmol. 2020 November; 48(8):1043-1056. doi: 10.1111/ceo.13834. Epub2020 Aug. 17. PMID: 32710488; PMCID: PMC7754492.
[18] Tarau I S, Berlin A, Curcio C A, Ach T. The Cytoskeleton of the Retinal Pigment Epithelium: from Normal Aging to Age-Related Macular Degeneration. Int J Mol Sci. 2019 Jul. 22; 20(14):3578. doi: 10.3390/ijms20143578. PMID: 31336621; PMCID: PMC6678077.
[20] Wyler S C, Lord C C, Lee S, Elmquist J K, Liu C. Serotonergic Control of Metabolic Homeostasis. Front Cell Neurosci. 2017 Sep. 20:11:277. doi: 10.3389/fncel.2017.00277. PMID: 28979187: PMCID: PMC5611374.
[21] Yongpeng Z, Yaxing W, Jingiong Z, Qian W. Yanni Y, Xuan Y, Jingyan Y, Wenjia Z, Ping W, Chang S, Ming Y, Yanan L, Jinyuan W, Shouling W, Shuohua C, Haiwei W, Lijian F, Qiangian W, Jingyuan Z, Zihan N, Yuning C, Ying X, Jonas J B, Wenbin W. The Association Between Diabetic Retinopathy and the Prevalence of Age-Related Macular Degeneration—The KailuanEye Study. Front Public Health. 2022 Jul. 18:10:922289. doi: 10.3389/fpubh.2022.922289. PMID: 35923972; PMCID: PMC9339787.
[22] Zhou R, Caspi R R. Ocular immune privilege. F1000 Biol Rep. 2010 Jan. 18; 2:3. doi: 10.3410/B2-3. PMID: 20948803; PMCID: PMC2948372.
[23] Zhu M, Liu X, Liu W, Lu Y, Cheng J, Chen Y. P cell aging and age-related diabetes. Aging (Albany NY). 2021 Mar. 3; 13(5):7691-7706. doi: 10.18632/aging.202593. Epub2021 Mar. 3. PMID: 33686020; PMCID: PMC7993693.

### Example 7

### Elucidating the Role of 5-HT in Mitochondrial Dynamics

### 5-HT Receptors—Background

7 isoforms of the GPCR serotonin receptor. Evolutionarily conserved in years and above. Associated with mood, appetite, sleep, and the immune system.

### Dynamic Detection of Mitochondria Via Red/Green Fluorescent Protein (FIG. 43)

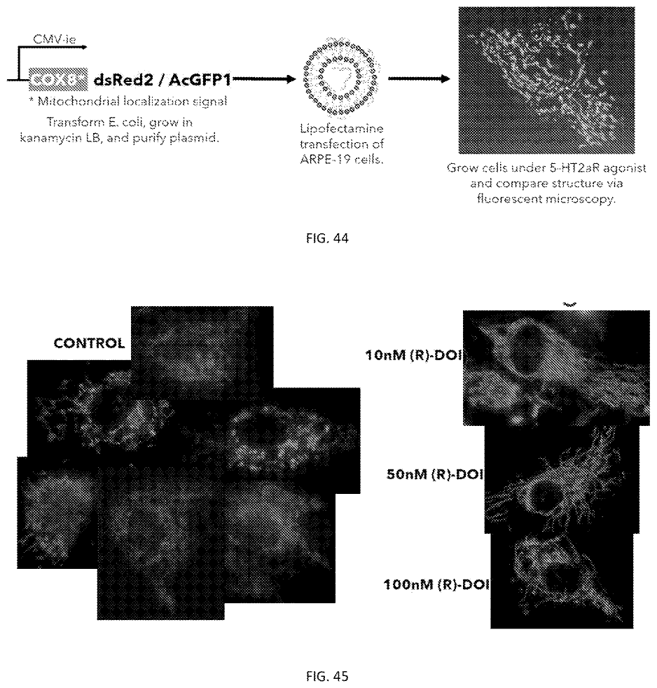

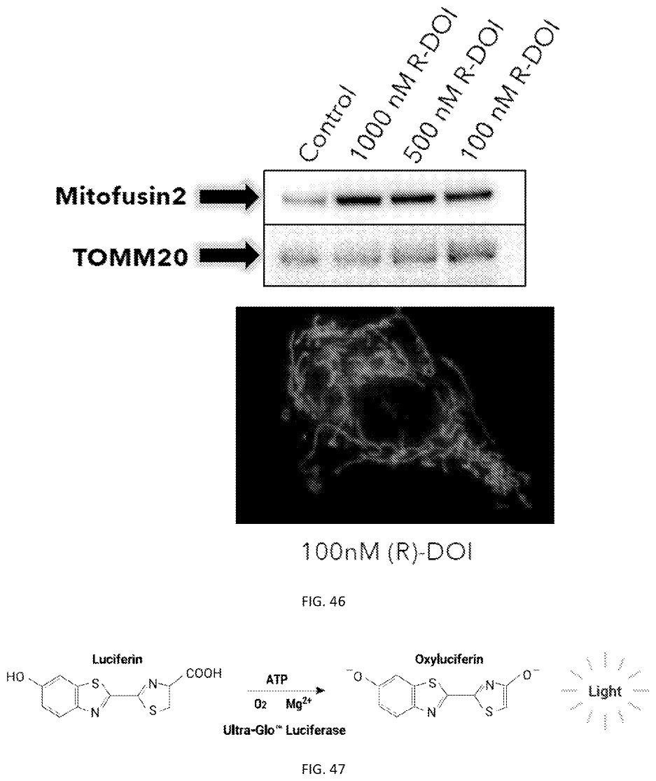

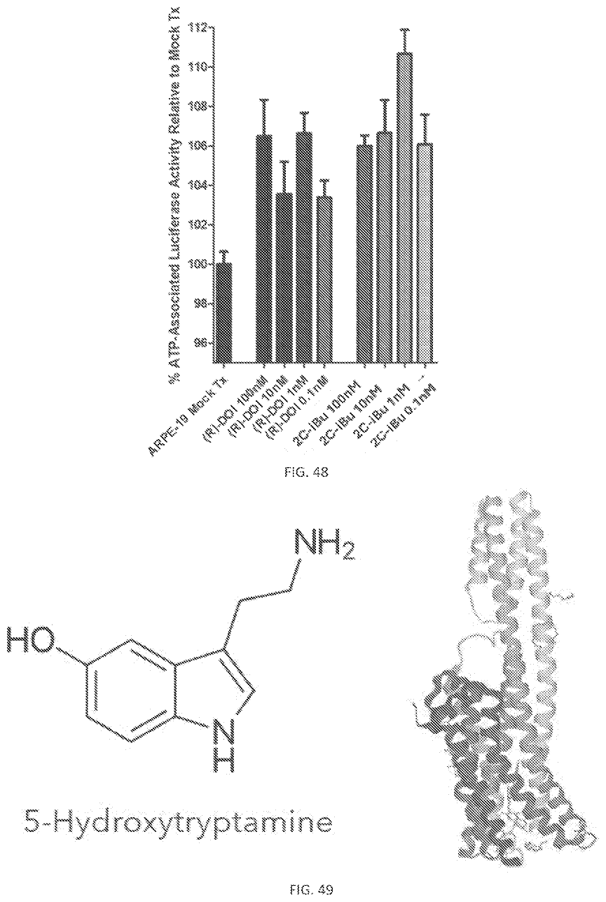

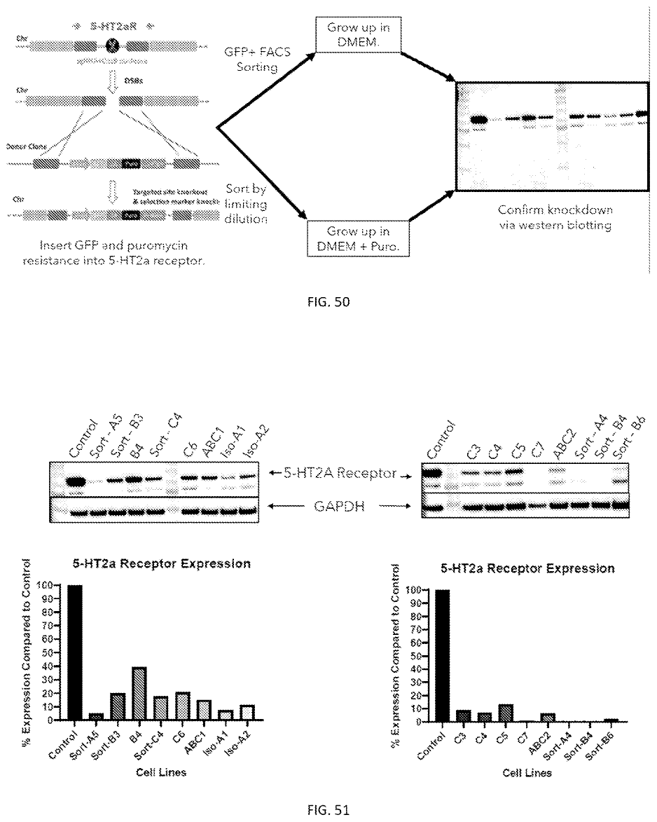

Fluorescent dyes limit researchers to study in a static manor due to fixation. We engineered a plasmid expressing mitochondrial localized AcGFP1/dsRed2. Expressing a red/green fluorescent protein within the mitochondria enables the researcher to dynamically study cells overtime.
Dynamic Detection of Mitochondria via Red/Green Fluorescent Protein (FIG. 44)
Fluorescently Labeled Mitochondria Reveal Structural Differences with 5-HT2AR Agonism (FIG. 45)
5-HT2 Receptor Agonism Upregulates Protein Involved in Biogenesis and Fusion (FIG. 46)
Mitofusion2—functions to regulate mitochondrial fusion
TOMM20—import of proteins through the outer membrane; marker of biogenesis and number
Analysis of Mitochondrial Function—ATP
The removal of ATP's tertiary PO₄ provides energy for a myriad enzymatic reactions within cells.
To quantify ATP, we use a luciferase assay to measure luminescence.
In this reaction, ATP hydrolysis fuels the conversion of luciferin to oxyluciferin.
The relative luminescence is proportional to the concentration of ATP within the cell (FIG. 47).
5-HT2A Receptor Agonism Increases ATP Production (FIG. 48)
Specific for 5-HT2A Receptor Agonism?
Development of a 5-HT2A Receptor Knockout
The effects and specificity of 5-HT2a receptor agonism can be further confirmed by comparisons with a knockout.
We will compare mitochondrial dynamics in cell-lines with varying expression of the 5-HT2a receptor.
5-HT2AR Knockout via CRISPR-Cas9 (FIG. 50)
Assessing 5-HT2A Receptor Knockout in ARPE-19 (FIG. 51)

### Percent of 5-HT2A Receptor Expression

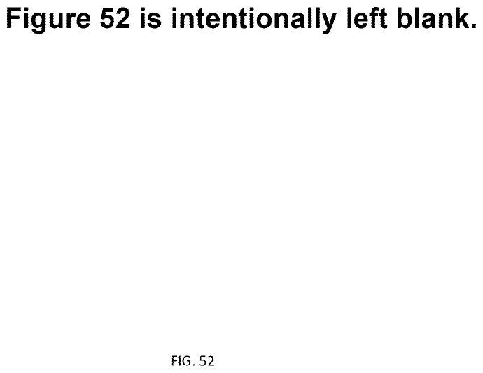

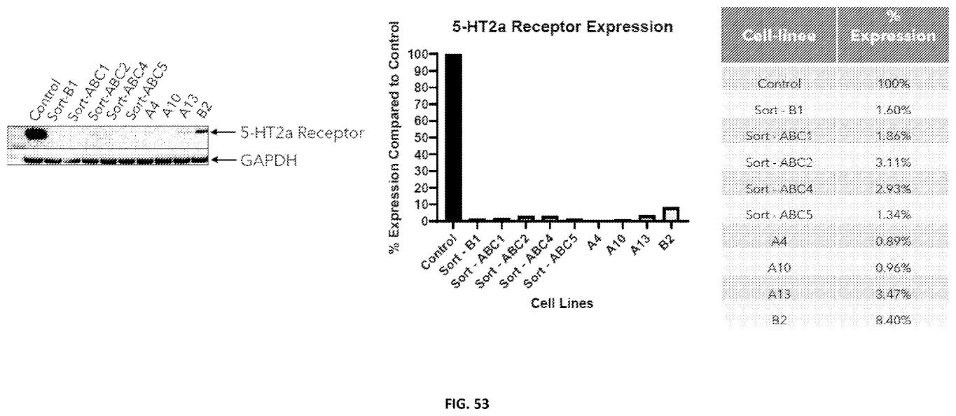

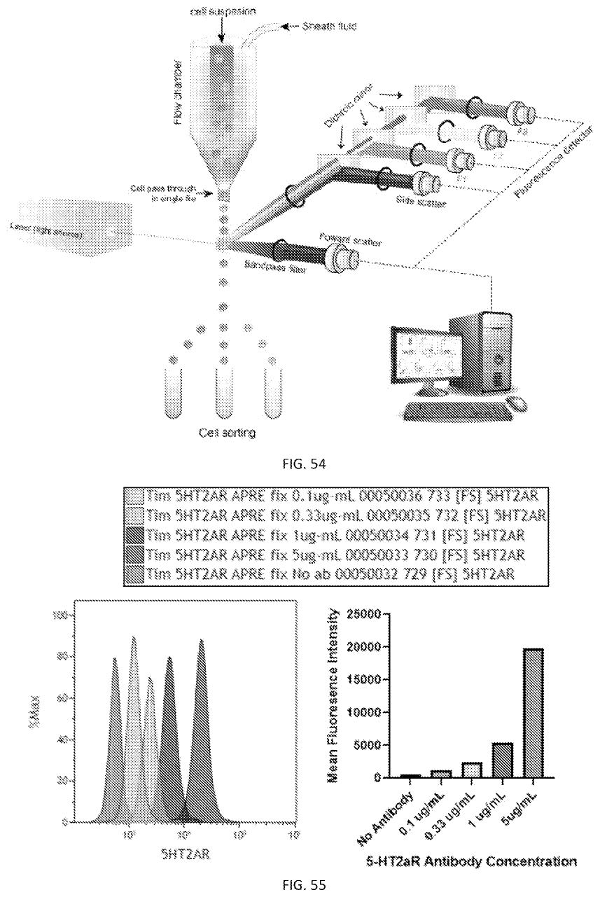

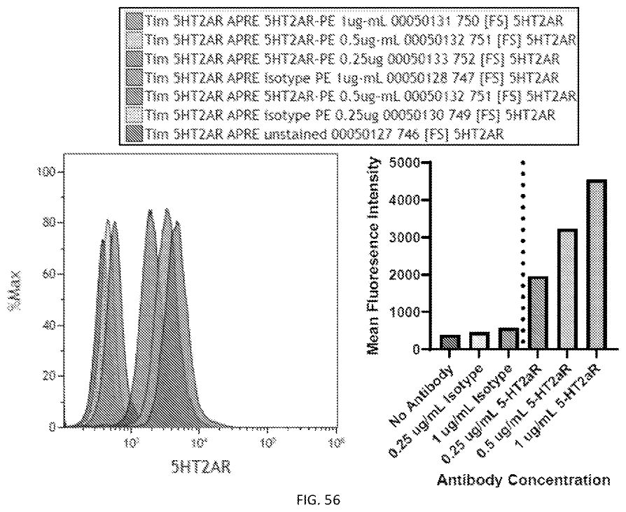

We have three >99% 5-HT2A receptor knockdowns and other cell-lines with varying expression (FIG. 52).
Additional 5-HT2A Receptor Knockouts
Interestingly, some of our best knockouts have taken the longest to grow. This can indicate the importance of the 5-HT2A receptor in mitochondrial dynamics and ATP production (FIG. 53).
Quantification of 5-HT2A Receptor by Flow (FIG. 54)
Western blotting tells us the average expression in a population of cells.
Flow cytometry tells us the expression in individual cells.
Optimizing 5-HT2AR Antibody Concentration (FIG. 55)
1 ug/mL provides adequate separation from the control.
Confirming Antibody Specificity with Isotype Control (FIG. 56)
Our isotype controls are well-separated from our samples.
Without wishing to be bound by theory, we can:
Compare 5-HT2a receptor knockouts with controls under 5-HT2a receptor agonism.
Quantify mitochondrial protein and transcripts involved in structure, function, and biogenesis through western blotting and SYBR Green RT-qPCR.
Evaluate transcriptional regulation through quantification of mitochondrial 7S RNA.
Investigate biomass through quantification of mitochondrial DNA.
Elucidate the mechanisms of cytoskeletal reorganization in knockouts.

### REFERENCES CITED IN THIS EXAMPLE

[1] Ding W X, Yin X M. Mitophagy: mechanisms, pathophysiological roles, and analysis. Biol Chem. 2012 July; 393(7):547-64. doi: 10.1515/hsz-2012-0119. PMID: 22944659; PMCID: PMC3630798.
[2] Mahmoudabadi G, Milo R, Phillips R. Energetic cost of building a virus. Proc Natl AcadSci USA. 2017 May 30; 114(22):E4324-E4333. doi: 10.1073/pnas.1701670114. Epub2017 May 16. PMID: 28512219; PMCID: PMC5465929.
[3] Moreno-Altamirano M M B, Kolstoe S E, Smnchez-Garcia F J. Virus Control of Cell Metabolism for Replication and Evasion of Host Immune Responses. Front Cell Infect Microbiol. 2019 Apr. 18; 9:95. doi: 10.3389/fcimb.2019.00095. PMID: 31058096; PMCID: PMC6482253.
[4] Summers W C. Virus Infection. Encyclopedia of Microbiology. 2009:546-52. doi: 10.1016/B978-012373944-5.00323-0. Epub2009 Feb. 17. PMCID: PMC7149757.
[5] Whitaker-Dowling P, Youngner J S. VIRUS-HOST CELL INTERACTIONS. Encyclopedia of Virology. 1999:1957-61. doi: 10.1006/rwvi.1999.0343. Epub2004 Jun. 17. PMCID: PMC7173423.
[6] Zhang L, Qin Y. Chen M. Viral strategies for triggering and manipulating mitophagy. Autophagy. 2018:14(10):1665-1673. doi: 10.1080/15548627.2018.1466014. Epub2018 Aug. 16. PMID: 29895192; PMCID: PMC6135629.

### Example 8

Without wishing to be bound by theory, the 5-HT2A receptor influences inflammatory and aging-associated diseases via modulation of the mTOR/AKT signaling pathways.

Without wishing to be bound by theory, we will:
1. Create 5-HT2A Receptor Knockdowns: Generate cell lines that specify 5-HT2A receptor knockdowns/knockouts via CRISPR/Cas9.
2. Characterize knockdowns and pharmacology. Characterize the morphological and functional consequences of 5-HT2A receptor modulation.
a. Pharmacological and physiological agonism/antagonism
b. Characterization of the 5-HT2A receptor genetic knockdown/knockout
3. Evaluate 5-HT2AR in Age-Related Diseases. Identify the effects of 5-HT2A receptor modulation on cellular processes associated with age-related diseases including cellular senescence and inflammation.
4. Define Molecular Mechanisms and Potential Therapeutic Targets. Evaluate the therapeutic potential of 5-HT2A receptor and mTOR/AKT modulation in age-related diseases.

Without wishing to be bound by theory, we can generate cell lines that specify 5-HT2A Receptor Knockdowns/Knockouts via CRISPR/Cas9

### Generating a 5-HT2A Receptor Knockdown in ARPE-19 (FIG. 50)

Western Blot Analysis: A Successful Knockdown of the 5-HT2A Receptor (FIG. 19). The pathophysiological impact of the Age-Associated Decline in Serotonin 2A Receptor Expression was assessed by Generating CRISPR/Cas9 Site-Directed Knockout of the Serotonin 2A Receptor in RPE cells. We generated 5-HT2AR knockouts via CRISPR/Cas9 in adult retinal pigmented epithelial cells from a healthy 19-yr old male (ARPE-19).

### Characterizing the Morphological and Functional Consequences of 5-HT2A Receptor Modulation

### Proliferation: The Role of Serotonin & 5-HT2AR in RPE Cell Replication

Physiologically relevant 5-HT levels increase ARPE-19 Proliferation
5-HT is an integral part of retinal development and homeostasis (FIG. 17).
Characterization: Pharmacological Agonism/Antagonism of the 5-HT2A Receptor Alters Proliferation in ARPE-19 (FIG. 18)
Characterization: 5-HT2AR Knockdowns Display Inhibited Proliferation Compared to the ARPE-19 WT (FIG. 20)

### Morphology: 5-HT2A Receptor Knockdown Increases Cell Size and Affects Cytoskeletal Structure

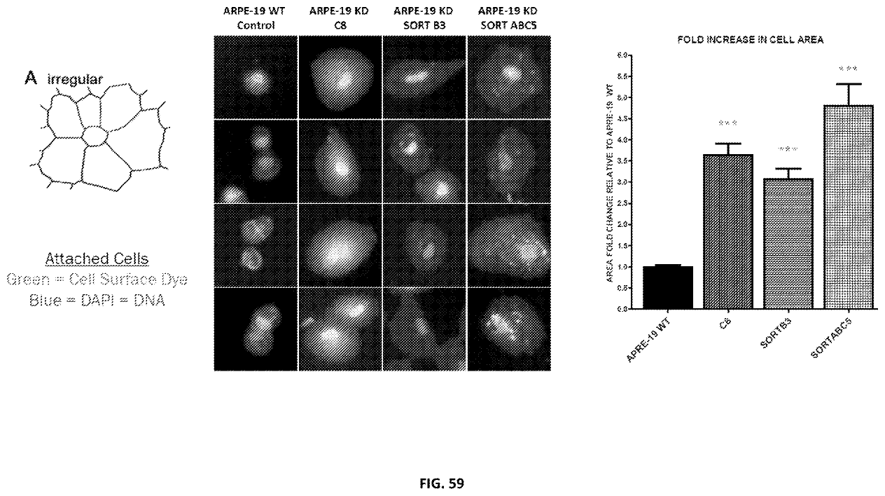

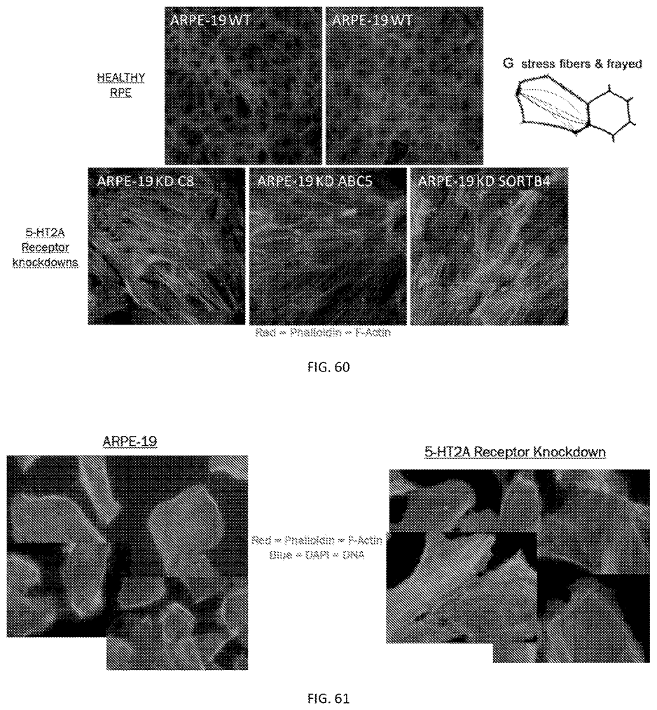

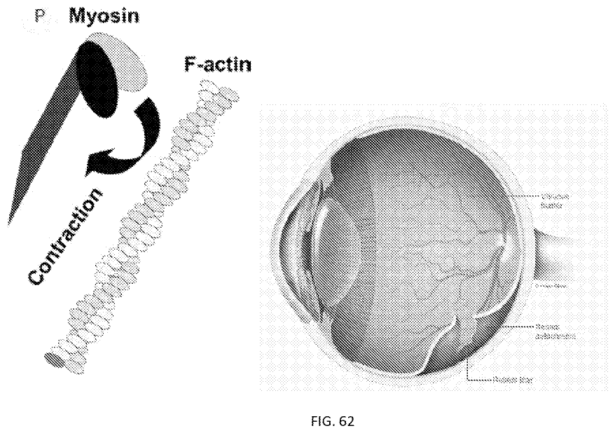

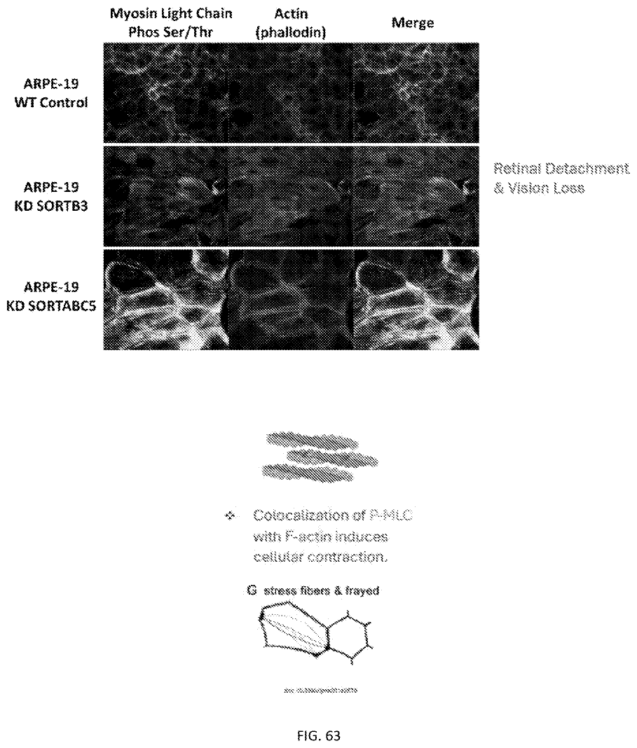

Morphological: 5-HT2A Receptor Knockdown Cells are Larger than the ARPRE-19 WT. We noticed that the 5-HT2A receptor knockdowns were strikingly larger than the ARPE-19 WT (FIG. 22).
RPE Size, Morphology, and Cytoskeletal Network is Impacted during AMD (FIG. 21)
Normal RPE are a single layer of highly-organized polygonal cells of similar size and shape.
Cytoskeletal elements influence cell size, morphology, subcellular trafficking, and multi-cellular communication.
The cytoskeletal network in the RPE is important for maintaining a healthy state of elasticity and permeability [18].
Cytoskeletal dysregulation impacts RPE integrity and is a well-documented feature of retinopathy, including AMD [18].
Morphological: 5-HT2A Receptor Knockdowns Have Increased Surface Area (FIG. 59 and FIG. 60)
Morphological: 5-HT2A Receptor Knockdowns have extensive actin stress fiber structures (FIG. 61).
Clinically, F-Actin stress fibers co-localize with phosphorylated myosin light chain Phosphorylated Myosin Light Chain (P-MLC) in AMD
P-MLC is a characteristic of AMD and other inflammatory diseases.
Phosphorylation of the MLC microfilament induces contraction.
A contracting retina can result in retinal detachment, the retina pulling away from the back of eye. This damage is accompanied by vision loss.
Morphological: Both P-Myosin Light Chain and F-Actin Stress Fiber Structures are Present in 5-HT2A Knockdowns (FIG. 63)
Function: RPE Cells Deficient in 5-HT2A Receptor Exhibit Lysosomal and Metabolic Dysregulation

### Functional: a Major Role of the RPE is to Keep the Eye Clean (FIG. 25)

The eye is unique in that it is considered immuno-privileged. The blood-retinal barrier prevents immune cell and macromolecule entry. The eye lacks efferent lymphatic vessels. Moreover, the ocular microenvironment is immunosuppressive via soluble factors and cell-to-cell interactions
The RPE is highly phagocytic, thus plays a major role in removing cellular debris in the eye through internalization and lysosomal degradation. Disruption of this process causes drusen deposition.

Morphological/Functional: 5-HT2A Receptor Knockdowns are more granular than the ARPE-19 WT. Increased cell granularity can be caused by lysosomal dysfunction, protein aggregation, and vacuolization. (FIG. 27).

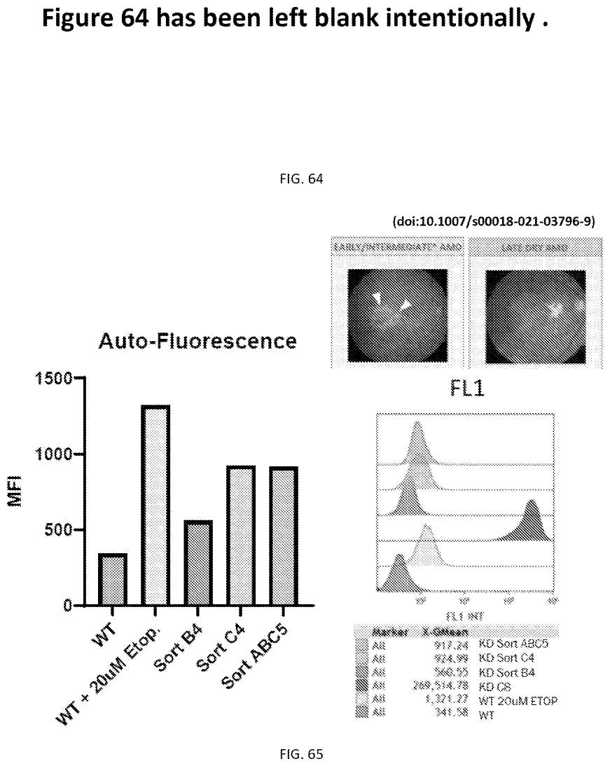

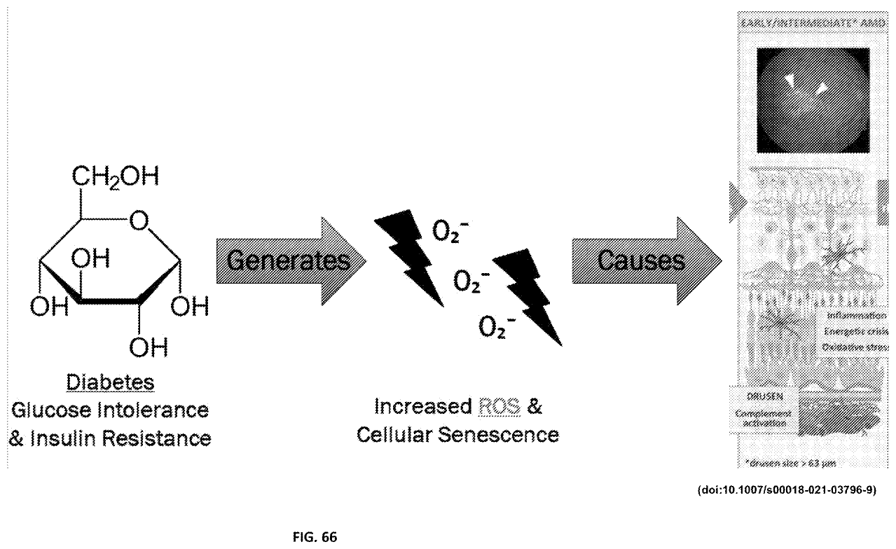

Lysotracker staining enables quantification of lysosomal content of a cell (FIG. 64). Lysotracker fluorophores are pH dependent probes which diffuse through membranes in their neutral state. Upon entry into the acidic lysosomal environment, the probe is protonated, becomes fluorescent, and loses membrane permeability.

5-HT2A Receptor knockdowns have more lysosomal content than the ARPE-19 WT. Lysosomal dysregulation is recognized as an important factor for age-related and neurodegenerative diseases (FIG. 28).

An auto-fluorescent phenotype is attributed to lysosomal lipofuscin accumulation (FIG. 65). Lipofuscin is a complex of lipids, protein, and co-factors that auto-fluoresce. Lipofuscin accumulation is potentially toxic (ROS) and a common feature of neurodegenerative, age-related pathology [10]. Lipofuscin accumulation facilitates drusen deposition, a major characteristic of AMD.

Lysosomal dysfunction and lipofuscin accumulation are associated with increased ROS levels (FIG. 30). Increased ROS levels are associated with metabolic dysfunction. Moreover, metabolic dysfunction causes cellular damage leading to the progression of age-related diseases, like AMD.

Metabolic Dysregulation in diabetes is a risk factor for AMD and other retinopathies.

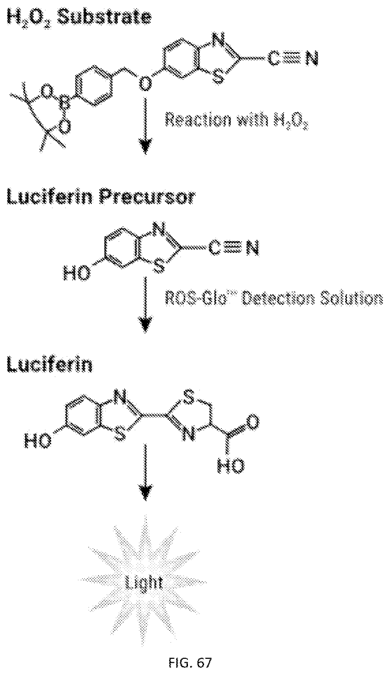

Evaluating intracellular reactive oxygen species (ROS) levels (FIG. 67). Promega's ROS-Glo H₂O₂ assay enables researchers to evaluate intracellular ROS levels. H₂O₂ is an ideal ROS to evaluate since it has the longest half-life, and many other ROS are converted into H₂O₂.

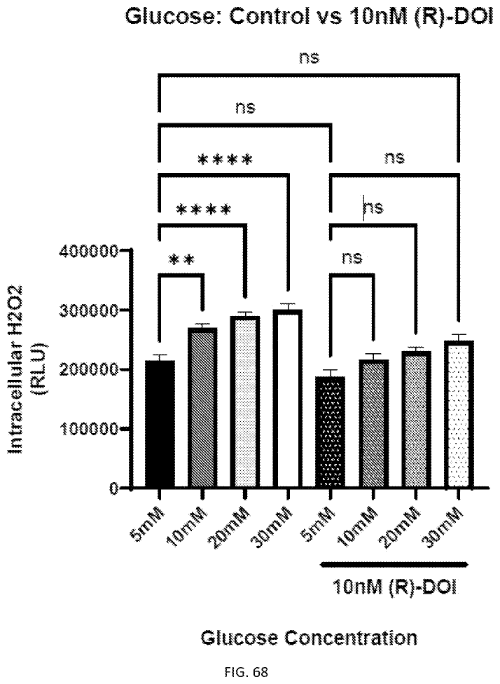

Pathological glucose concentrations induce ROS and this is alleviated by 5-HT2A receptor agonism (FIG. 68). Physiological media with increasing glucose levels. Pathological glucose concentrations increase intracellular H₂O₂. 5-HT2AR agonism alleviates glucose-induced intracellular H2O2 at diabetic glucose concentrations.

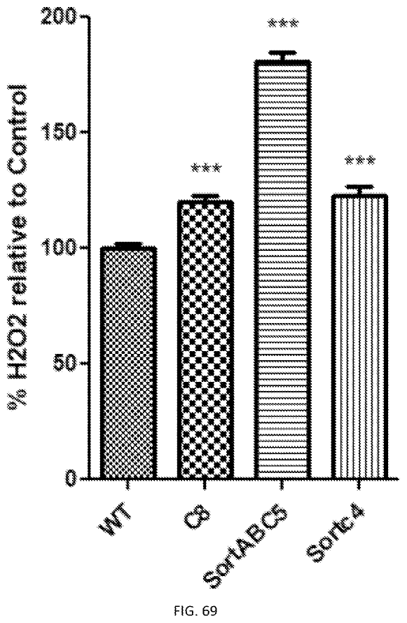

Functional: 5-HT2A Receptor knockdowns have higher levels of intracellular ROS. Increased ROS can cause damage to DNA, protein, and lipids which are characteristic of age-related diseases, including AMD (FIG. 69).

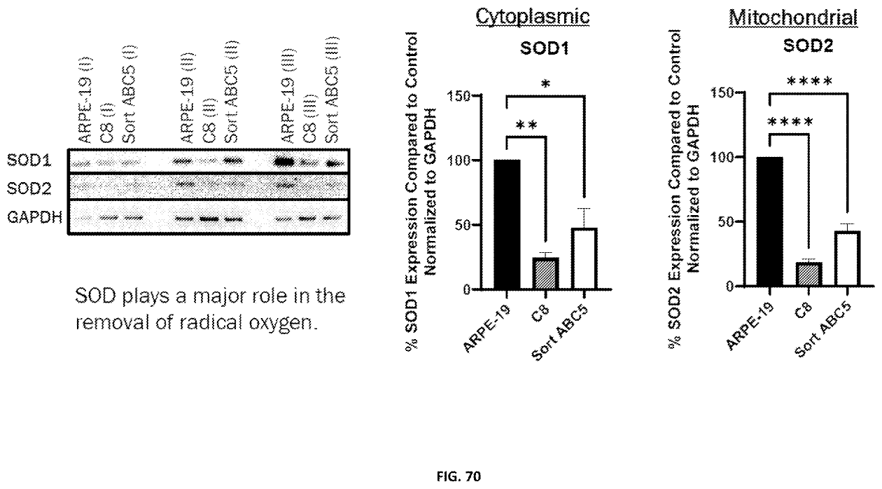

Functional: 5-HT2A receptor knockdowns have lower expression of cytoplasmic/mitochondrial superoxide dismutase (SOD) antioxidants (FIG. 70).

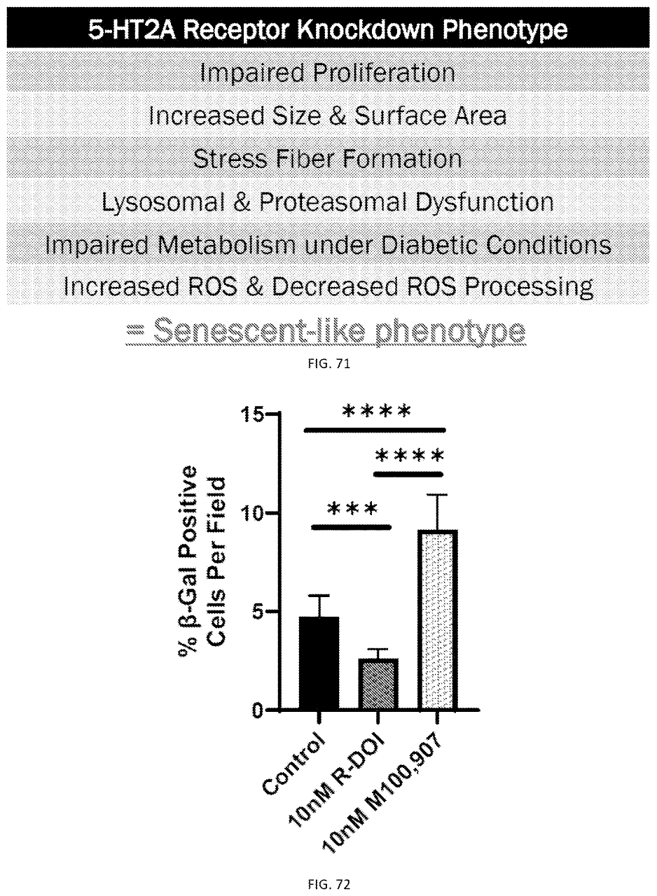

Without wishing to be bound by theory, we can identify the effects of 5-HT2A receptor modulation on cellular processes associated with age-related diseases, included cellular senescence, and inflammation. FIG. 71 shows a schematic which indicates characteristics of a “Senescent-like phenotype.”

What is cellular senescence? Senescence is the cellular equivalent to aging. Senescence is defined as ‘irreversible’ cell-cycle arrest. Senescent cell characteristics include limited proliferation, enlarged cell size, excessive ROS, metabolic and proteolytic dysfunction, senescence-associated secretory phenotype (pro-inflammatory cytokines), nutrient imbalances, telomere attrition, DNA damage, and epigenetic dysregulation [17]. In addition to these characteristics, senescence-associated β-galactosidase(SA-β-Gal) staining is the gold standard for quantifying cellular senescence.

Nutrient rich conditions increase levels of senescence-associated beta-galactosidase in ARPE-19 (FIG. 36).

The ARPE-19 senescent-like phenotype is reduced in physiological media compared to DMEM/F-12 (FIG. 37). Human plasma-like media (HPLM) is a media comparable to physiological conditions. DMEM/F-12 is a nutrient rich media which has increased glucose, pyruvate, and other nutrients.

The senescent-like phenotype increases with increasing glucose concentrations in physiological media (FIG. 38). Pathological glucose concentrations induce cellular senescence. This mirrors the ROS data. 5-HT2A receptor agonism alleviates glucose-induced senescence at diabetic blood glucose levels.

5-HT2AR Antagonism increases the senescent-like phenotype and agonism reduces the senescent-like phenotype in the ARPE-19 WT (FIG. 72).

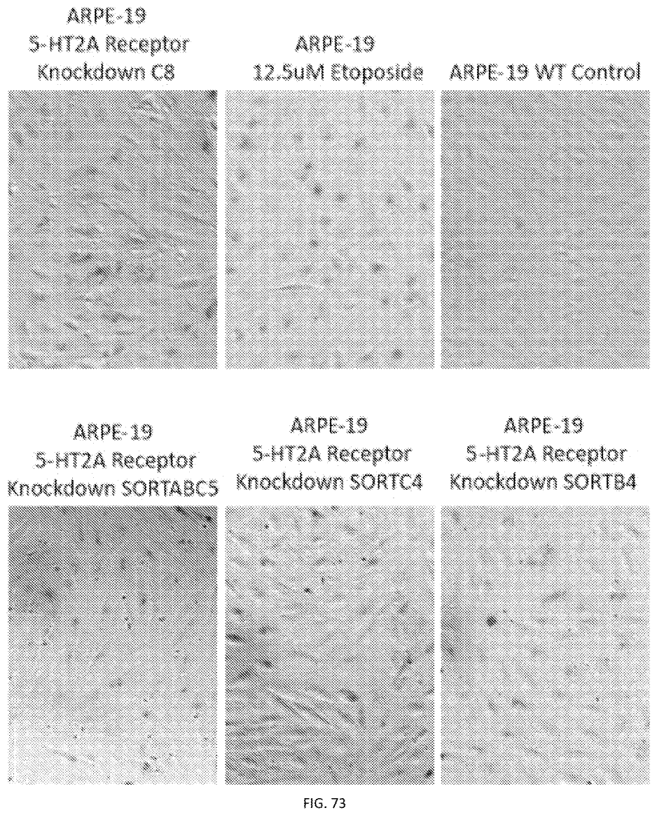

5-HT2a receptor knockdowns have high expression of B-Gal compared to the ARPE-19 Control (FIG. 73).

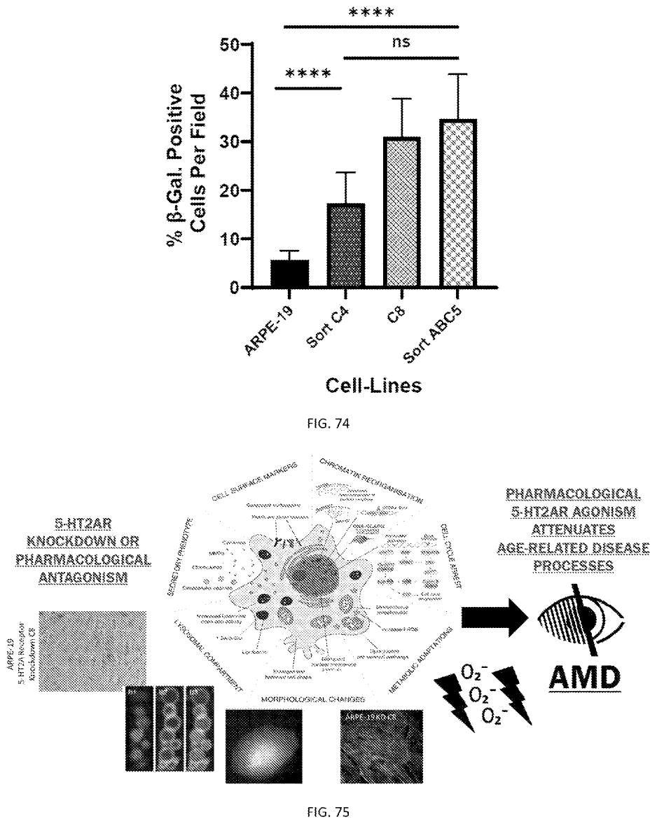

5-HT2A Receptor knockdowns have high expression of B-gal compared to the ARPE-19 Control (FIG. 74).

### REFERENCES CITED IN THIS EXAMPLE

[1] Gregg E W, Engelgau M M, Narayan V. Complications of diabetes in elderly people. BMJ. 2002 Oct. 26; 325(7370):916-7. doi: 10.1136/bmj.325.7370.916. PMID: 12399324; PMCID: PMC1124430.
[2] Armento A, Ueffing M. Clark S J. The complement system in age-related macular degeneration. Cell Mol Life Sci. 2021 May; 78(10):4487-4505. doi: 10.1007/s00018-021-03796-9. Epub2021 Mar. 9. PMID: 33751148; PMCID: PMC8195907.
[3] Azmitia E C. Serotonin neurons, neuroplasticity, and homeostasis of neural tissue. Neuropsychopharmacology. 1999 August; 21(2 Suppl):33S-45S. doi: 10.1016/S0893-133X(99)00022-6. PMID: 10432487.
[4] Chen X, Rong S S, Xu Q, Tang F Y, Liu Y, Gu H, Tam P O, Chen L J, Brelen M E, Pang C P, Zhao C. Diabetes mellitus and risk of age-related macular degeneration: a systematic review and meta-analysis. PLoSOne. 2014 Sep. 19; 9(9):e108196. doi: 10.1371/joumal.pone.0108196. PMID: 25238063; PMCID: PMC4169602.
[5] Gilbert S F. Developmental Biology. 6th edition. Sunderland (MA): Sinauer Associates; 2000. Aging: The Biology of Senescence.
[6] Karrer T M, McLaughlin C L, Guaglianone C P, Samanez-Larkin G R. Reduced serotonin receptors and transporters in normal aging adults: a meta-analysis of PET and SPECT imaging studies. NeurobiolAging. 2019 August:80:1-10. doi: 10.1016/j.neurobiolaging.2019.03.021. Epub2019 Apr. 5. PMID: 31055162: PMCID: PMC6679764.
[7] Lu T C, Brbid M, Park Y J, Jackson T, Chen J. Kolluru S S, Qi Y, Katheder N S, Cai X T, Lee S, Chen Y C, Auld N, Liang C Y, Ding S H, Welsch D, D'Souza S. Pisco A O, Jones R C, Leskovec J, Lai E C, Bellen H J, Luo L, Jasper H, Quake S R, Li H. Aging Fly Cell Atlas identifies exhaustive aging features at cellular resolution. Science. 2023 Jun. 16; 380(6650):eadg0934. doi: 10.1126/science.adg0934. Epub2023 Jun. 16. PMID: 37319212.
[8] Mazzoni F, Safa H, Finnemann S C. Understanding photoreceptor outer segment phagocytosis: use and utility of RPE cells in culture. Exp Eye Res. 2014 September; 126:51-60. doi: 10.1016/j.exer.2014.01.010. Epub2014 Apr. 26. PMID: 24780752: PMCID: PMC4145030.
[9] McHugh D, Gil J. Senescence and aging: Causes, consequences, and therapeutic avenues. J Cell Biol. 2018 Jan. 2; 217(1):65-77. doi: 10.1083/jcb.201708092. Epub2017 Nov. 7. PMID: 29114066; PMCID: PMC5748990.
[10] Moiseiwitsch J R, Lauder J M. Serotonin regulates mouse cranial neural crest migration. Proc Natl AcadSci USA. 1995 Aug. 1; 92(16):7182-6. doi: 10.1073/pnas.92.16.7182. PMID: 7638165; PMCID: PMC41303.
[11] Moreno-Garcia A, Kun A, Calero O, Medina M, Calero M. An Overview of the Role of Lipofuscin in Age-Related Neurodegeneration. Front Neurosci. 2018 Jul. 5:12:464. doi: 10.3389/fnins.2018.00464. PMID: 30026686: PMCID: PMC6041410.
[12] Morgan D G. The dopamine and serotonin systems during aging in human and rodent brain. A brief review. Prog NeuropsychopharmacolBiol Psychiatry. 1987; 11(2-3):153-7. doi: 10.1016/0278-5846(87)90053-4. PMID: 2819950.
[14] PootanakitK, Prior K J, Hunter D D, Brunken W J. 5-HT2a receptors in the rabbit retina: potential presynaptic modulators. Vis Neurosci. 1999 March-April; 16(2):221-30. doi: 10.1017/s0952523899162035. PMID: 10367957.
[15] Sanada F, Taniyama Y, Muratsu J, Otsu R, Shimizu H, Rakugi H, Morishita R. Source of Chronic Inflammation in Aging. Front Cardiovasc Med. 2018 Feb. 22; 5:12. doi: 10.3389/fcvm.2018.00012. PMID: 29564335; PMCID: PMC5850851.
[16] Sharif N A. iDrugs and iDevicesDiscovery Research: Preclinical Assays, Techniques, and Animal Model Studies for Ocular Hypotensives and Neuroprotectants. J OculPharmacolTher. 2018 January/February; 34(1-2):7-39. doi: 10.1089/jop.2017.0125. Epub2018 Jan. 11. PMID: 29323613.
[17] Somasundaran S, Constable I J, Mellough C B, Carvalho L S. Retinal pigment epithelium and age-related macular degeneration: A review of major disease mechanisms. Clin Exp Ophthalmol. 2020 November; 48(8):1043-1056. doi: 10.1111/ceo.13834. Epub2020 Aug. 17. PMID: 32710488; PMCID: PMC7754492.
[18] Tarau I S, Berlin A, Curcio C A, Ach T. The Cytoskeleton of the Retinal Pigment Epithelium: from Normal Aging to Age-Related Macular Degeneration. Int J Mol Sci. 2019 Jul. 22; 20(14):3578. doi: 10.3390/ijms20143578. PMID: 31336621; PMCID: PMC6678077.
[20] Wyler S C, Lord C C, Lee S, Elmquist J K, Liu C. Serotonergic Control of Metabolic Homeostasis. Front Cell Neurosci. 2017 Sep. 20:11:277. doi: 10.3389/fncel.2017.00277. PMID: 28979187; PMCID: PMC5611374.
[21] Yongpeng Z, Yaxing W, Jingiong Z, Qian W, Yanni Y, Xuan Y, Jingyan Y, Wenjia Z, Ping W, Chang S, Ming Y, Yanan L, Jinyuan W, Shouling W, Shuohua C, Haiwei W, Lijian F, Qianqian W, Jingyuan Z, Zihan N, Yuning C, Ying X, Jonas J B, Wenbin W. The Association Between Diabetic Retinopathy and the Prevalence of Age-Related Macular Degeneration—The KailuanEye Study. Front Public Health. 2022 Jul. 18:10:922289. doi: 10.3389/fpubh.2022.922289. PMID: 35923972; PMCID: PMC9339787.
[22] Zhou R, Caspi R R. Ocular immune privilege. F1000 Biol Rep. 2010 Jan. 18; 2:3. doi: 10.3410/B2-3. PMID: 20948803; PMCID: PMC2948372.
[23] Zhu M, Liu X. Liu W, Lu Y, Cheng J, Chen Y. β cell aging and age-related diabetes. Aging (Albany NY). 2021 Mar. 3; 13(5):7691-7706. doi: 10.18632/aging.202593. Epub2021 Mar. 3. PMID: 33686020; PMCID: PMC7993693.

### Example 9

### The Role of the Serotonin 2A Receptor (5-HT2AR) in Retinal Pigmented Epithelial Cell-Associated Development and Progression of Age-Related Macular Degeneration

Age-related macular degeneration (AMD) is characterized by disruption of the retinal pigmented epithelium (RPE) (FIG. 13).

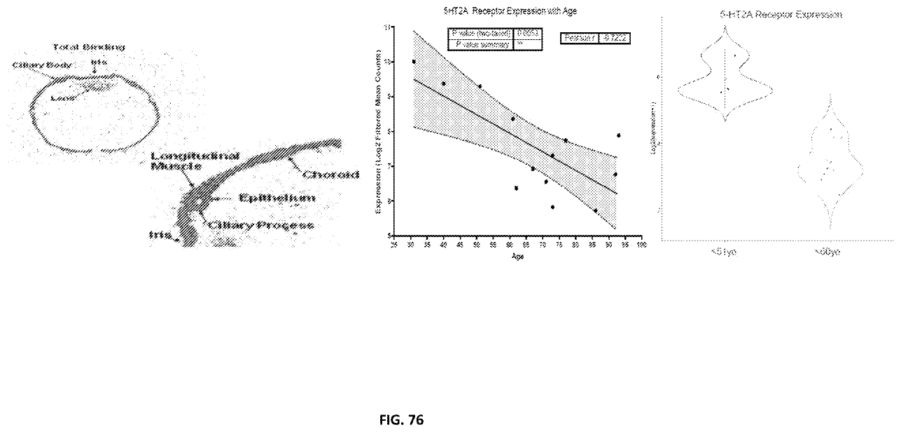

The serotonergic system affects age-related disease processes (FIG. 76). Serotonin regulates central metabolism, insulin sensitivity, the vasculature, and inflammation. The serotonin 2A receptor (5-HT2AR) is highly expressed and localized in the RPE. Moreover, 5-HT2AR expression declines with age.

Without wishing to be bound by theory, 5-HT2AR repression contributes to disease processes associated with AMD progression.

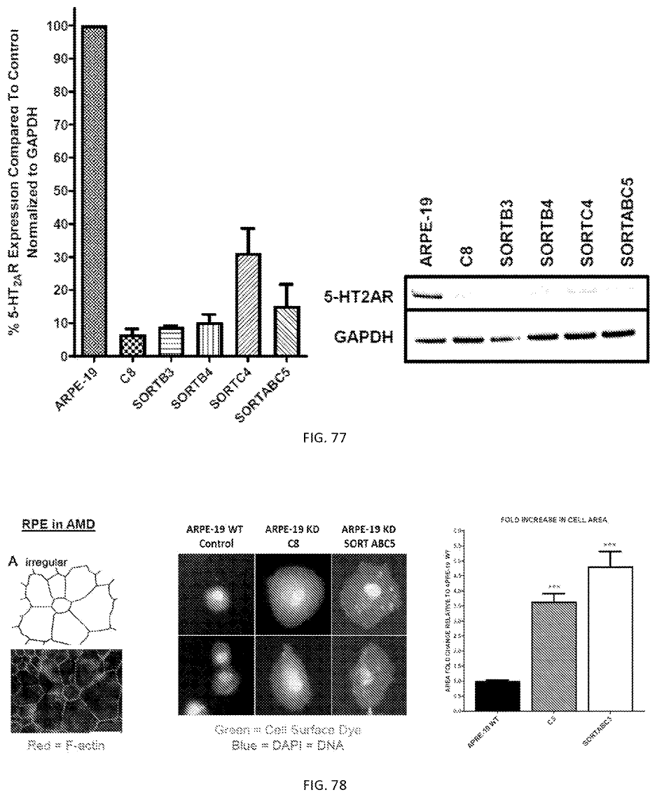

To facilitate assessment of the role of 5-HT2AR in age-related diseases of the eye, we generated 5-HT2AR knockouts in ARPE-19 cells (FIG. 77). We generated 5-HT2AR knockouts via CRISPR/Cas9 in adult retinal pigmented epithelial cells from a healthy 19-year-old male (ARPE-19).

5-HT2AR Knockouts exhibit increased cellular area consistent with dysfunctional RPE in AMD (FIG. 78).

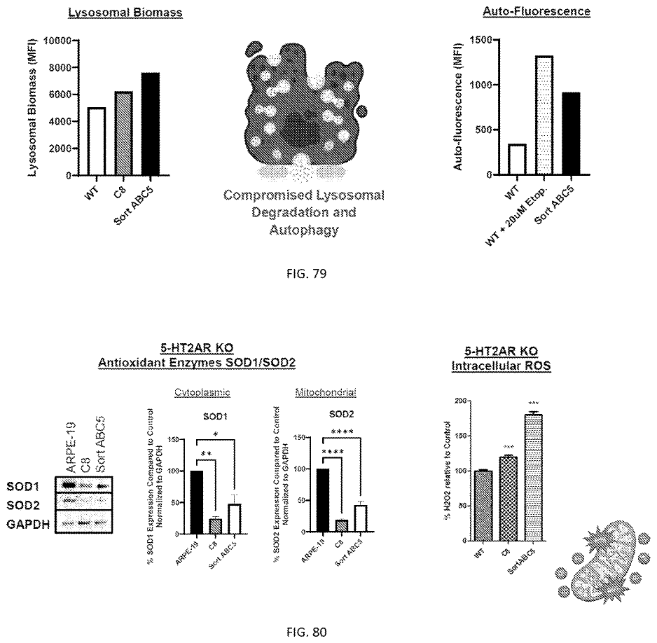

5-HT2AR Kos have increased lysosomal biomass and are auto-fluorescent, indicative of lysosomal dysfunction and lipofuscin accumulation (FIG. 79).

5-HT2AR Kos exhibit decreased expression of cytoplasmic and mitochondrial antioxidants and increased levels of ROS (FIG. 80).

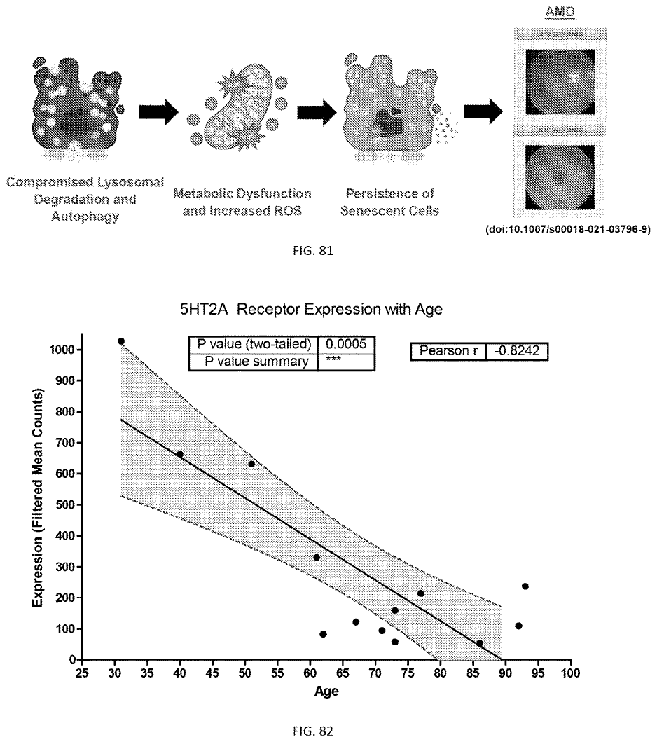

ROS is a driver of cellular senescence in AMD and other age-related diseases (FIG. 81).

5-HT2AR Knockout or pharmacological antagonism induces cellular senescence (FIGS. 34 and 35).

### Example 10

5HT2A Receptor Expression Levels within the Retina STRONGLY & Negatively Correlates with Age: Linear Filtered Counts (FIG. 82).

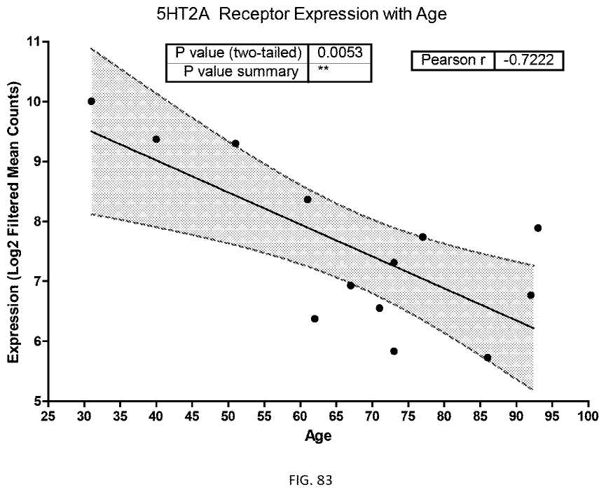

5HT2A Receptor Expression Levels within the Retina STRONGLY & Negatively Correlates with Age: Log 2 Filtered Mean Counts (FIG. 83).

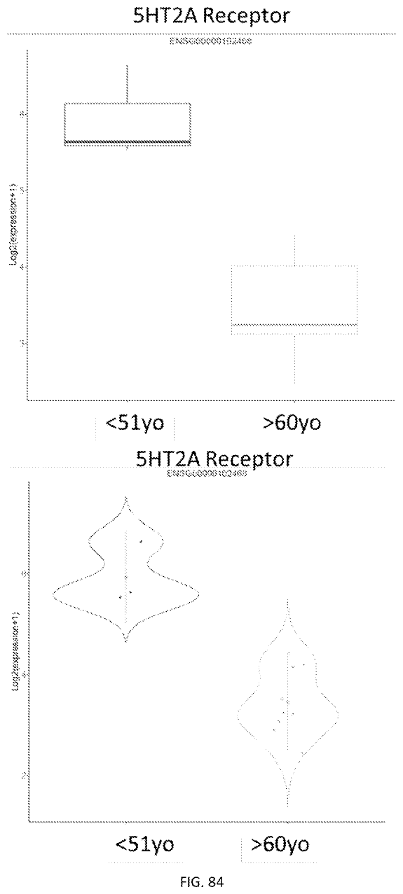

5HT2A Receptor Expression is Significantly Decreased within the Retina after 60 Years of Age (FIG. 84).

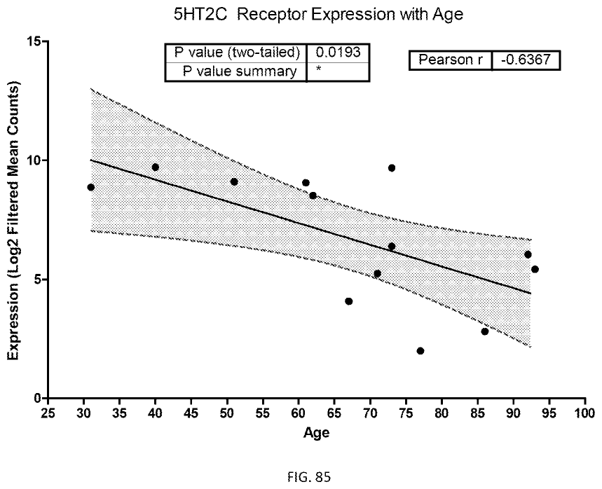

Expression of 5HT2C Receptor Expression Levels within the Retina Negatively Correlates with Age; However, Changes are not as Significant or as Strong as with 5HT2A Receptor (FIG. 85).

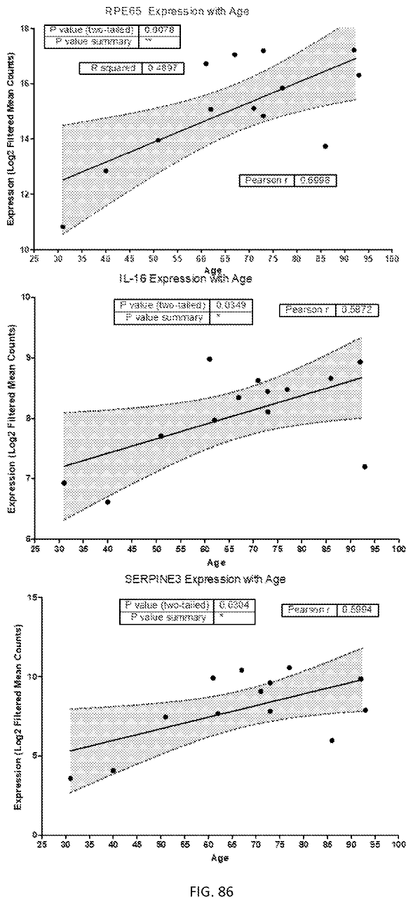

As Expression of 5HT2A Receptor Expression Levels Decreases within the Retina Expression of Genes that Influence Retinal Metabolism & Inflammation Increase with Age (FIG. 86).

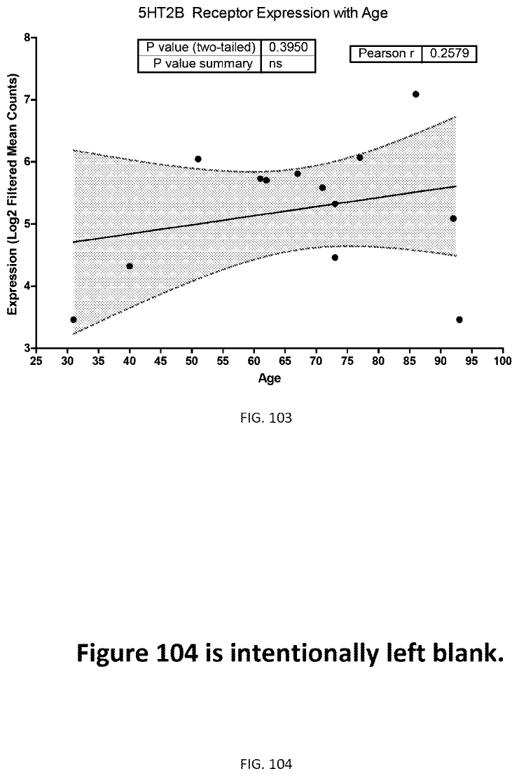

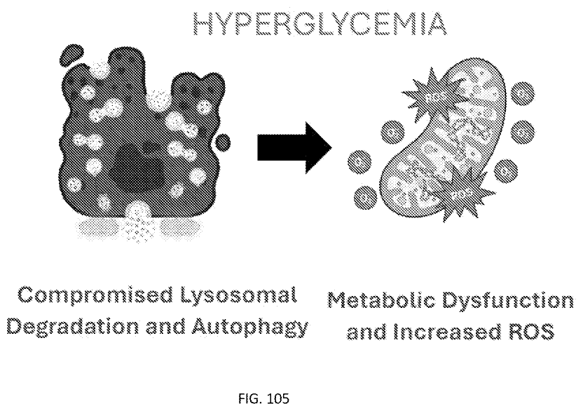

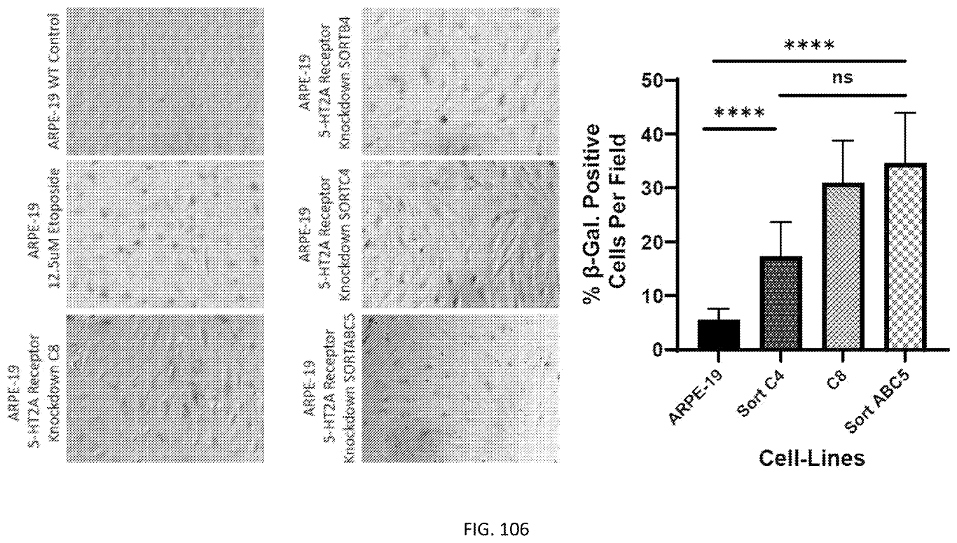

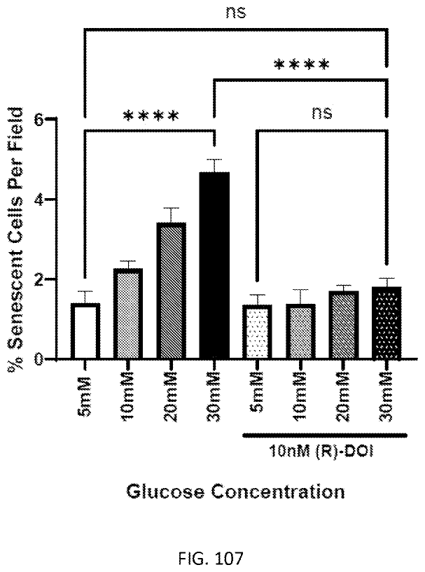

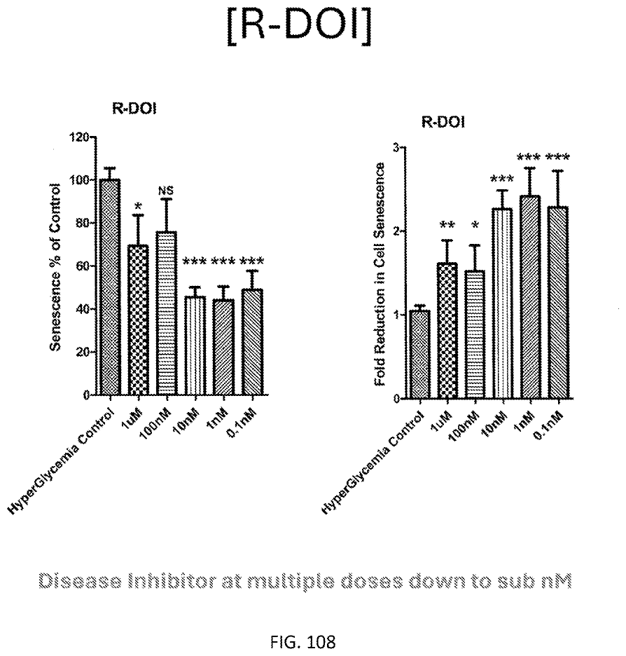

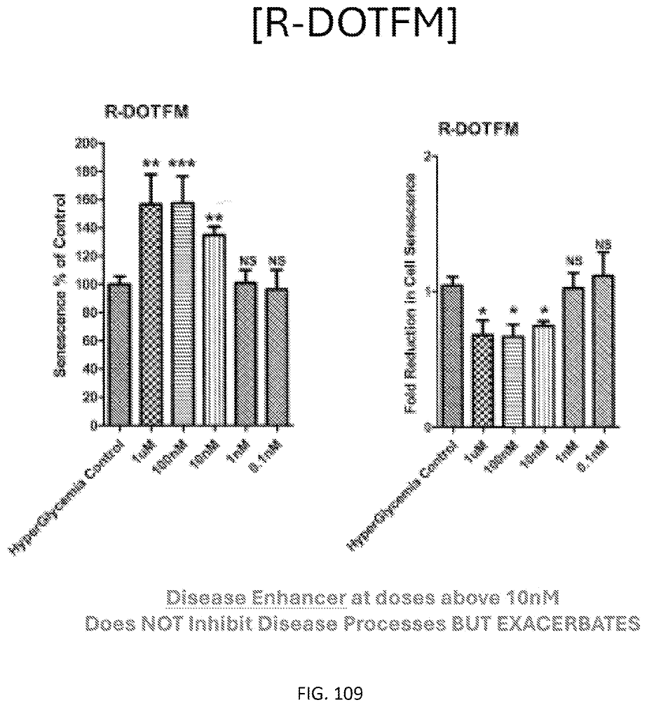

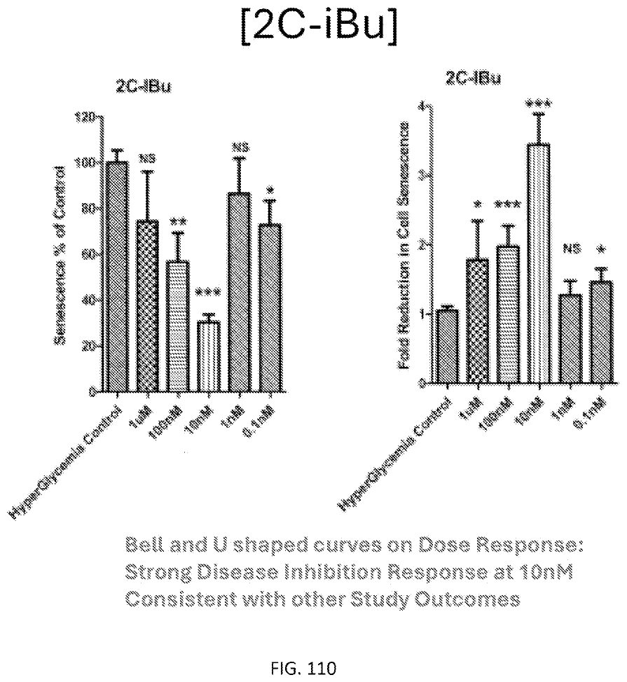

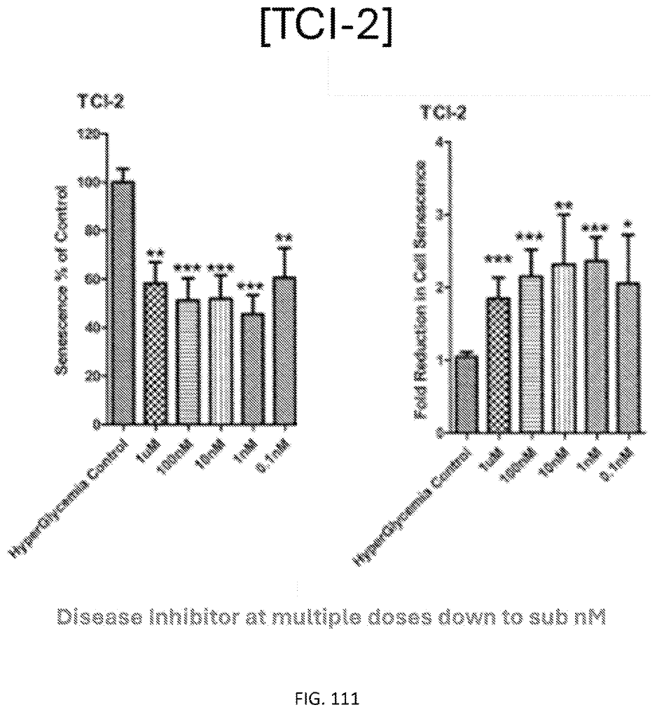

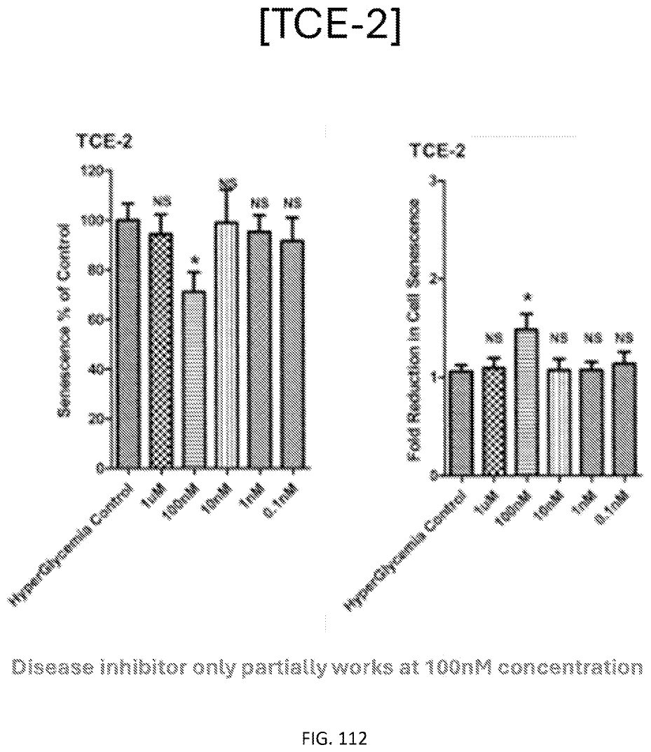

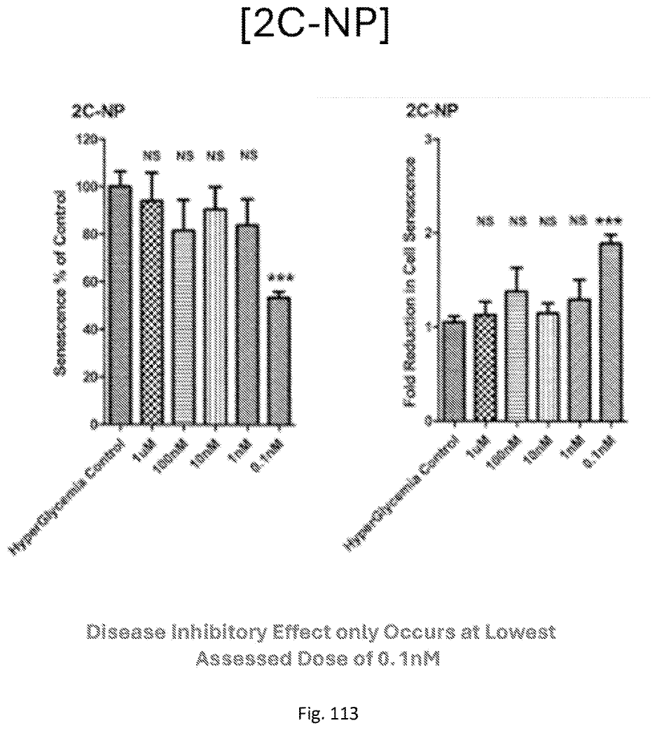

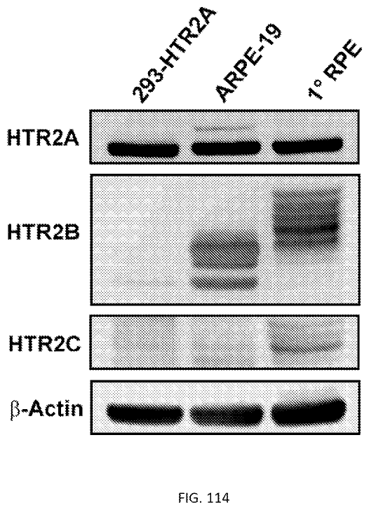

Expression of other serotonin receptors, including 5-HT2B within the Retina DO NOT correlate with age: Log 2 mean counts (FIG. 103).

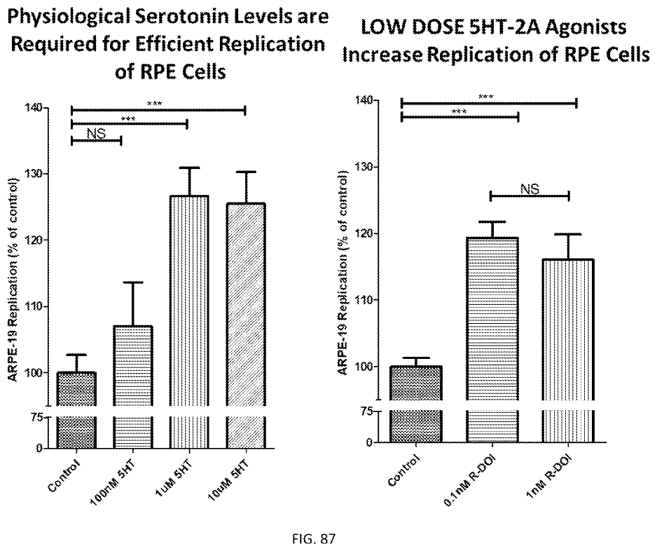

5-HT2A Receptor Function is Required for Healthy Replication of Retinal Pigmented Epithelia: Serotonin Enhances Cellular Health and Replication through the 5-HT2A Receptor (FIG. 87).

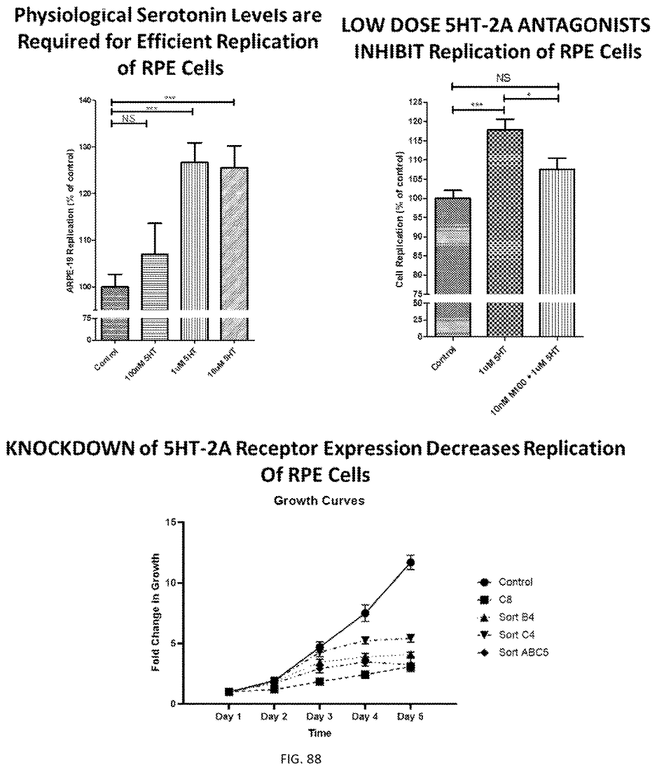

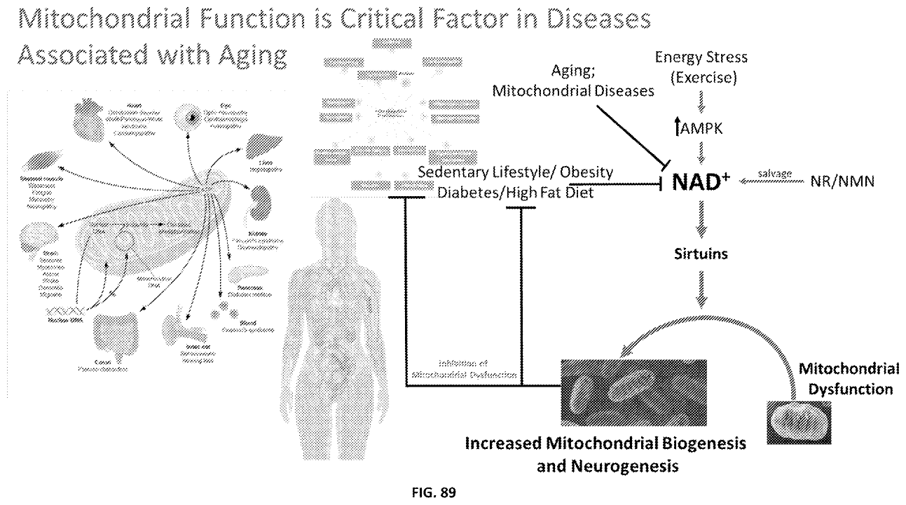

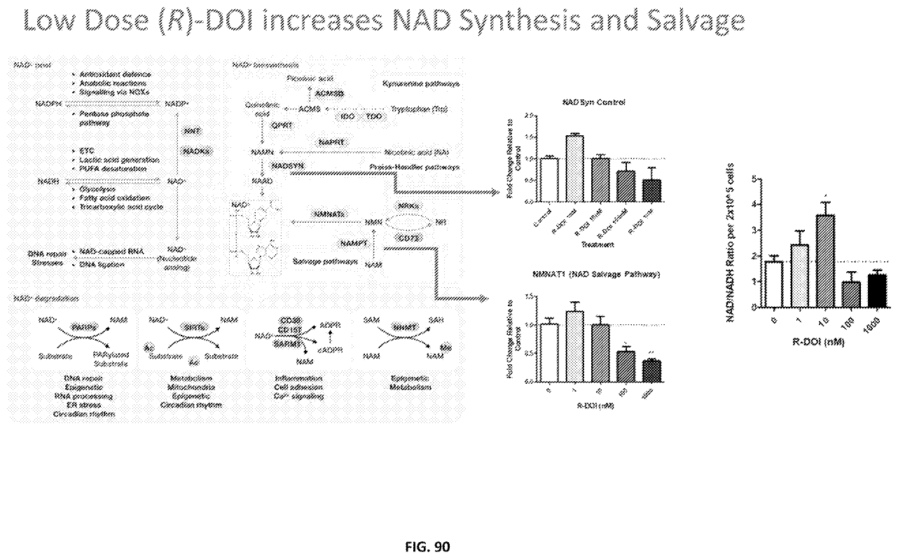

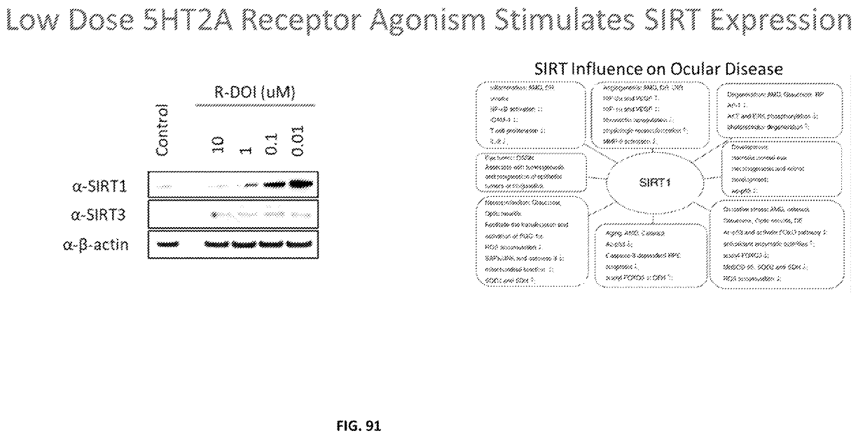

5-HT2A Receptor Function is Required for Healthy Replication of Retinal Pigmented Epithelia (FIG. 88). In the absence of the 5-HT2A receptor, cell health and proliferation are reduced. This is consistent with the Age-Related Declines in Repair of the Blood-Retinal Barrier in AMD, Diabetic Retinopathy, and Other Retinopathies.

### Epigenic Regulation—SIRT1

SIRT1 expression inhibits both peripheral and neuroinflammation and modulates neuroplasticity in vitro and in vivo.
Increasing SIRT1 expression promotes mitochondrial function, the autophagic clearance of damaged mitochondria and effective stress adaptation.
Increasing SIRT1 indirectly enhances AMPK signaling impaired in insulin resistance, reduces oxidative stress, and promotes neuronal growth and survival.
Inhibition of SIRT1 results in insulin resistance in animals while upregulation of SIRT1 enhances insulin sensitivity and reduces insulin resistance-associated inflammation.
2A agonists accelerate the recovery of mitochondrial function after oxidant-induced injury in vitro, demonstrating the capacity to mediate regulatory control of mitochondrial biogenesis.
2A agonists facilitate mitochondrial biogenesis and neuroprotection by upregulation of SIRT1 mRNA and protein, and also increases SIRT1-PGC-1a functional expression and associated upregulation of mitochondrial function and biogenesis in the cortex.

### 5HT2A Receptor Agonism Enhances Mitochondrial Function

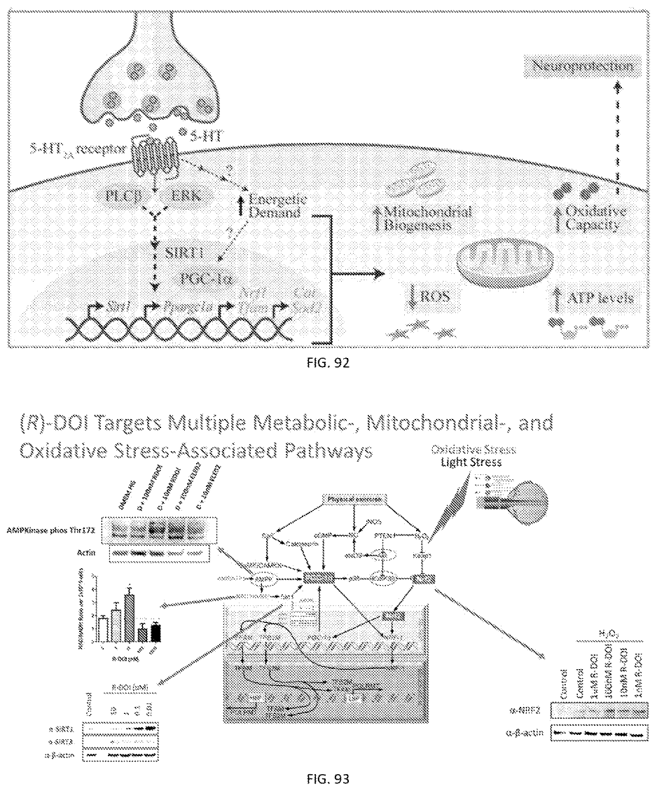

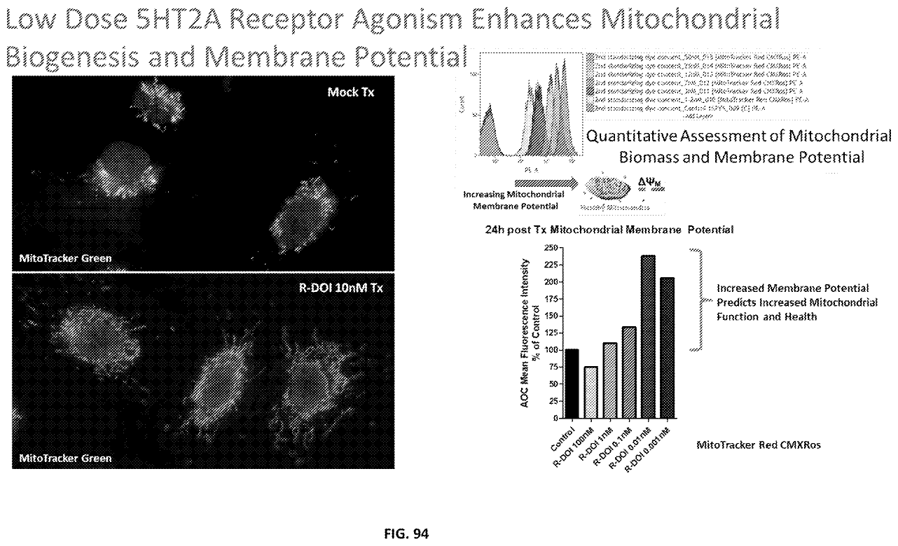

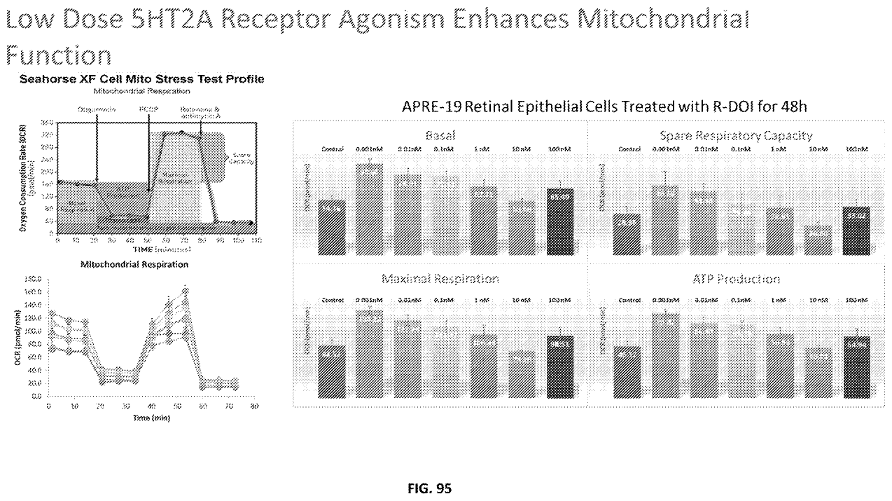

Epigenetic regulation—SIRT1
(R)-DOI upregulates SIRT1 which is anti-inflammatory and enhances mitochondrial biogenesis and cellular survival under stress (FIG. 92).
Low Dose 5HT2A Receptor Agonism Enhances Mitochondrial Biogenesis and Membrane Potential (FIG. 93).
Low Dose 5HT2A Receptor Agonism Enhances Mitochondrial Function (FIG. 95).

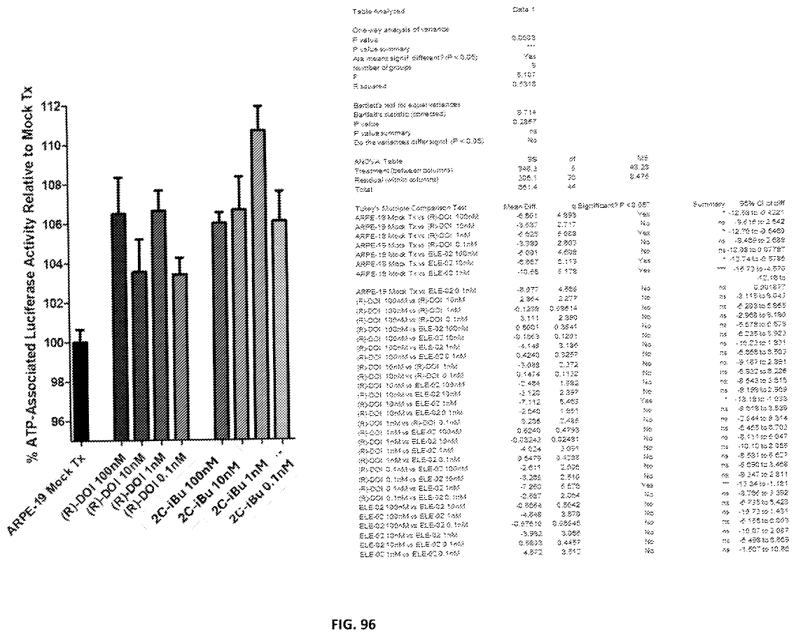

### 5-HT2A Receptor Agonists Increase Mitochondrial Function And ATP Production (FIG. 96).

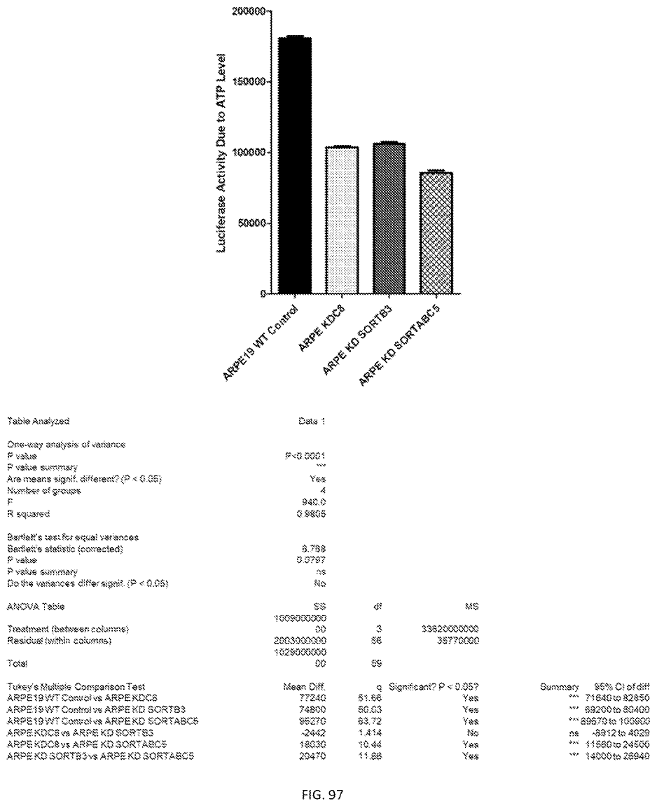

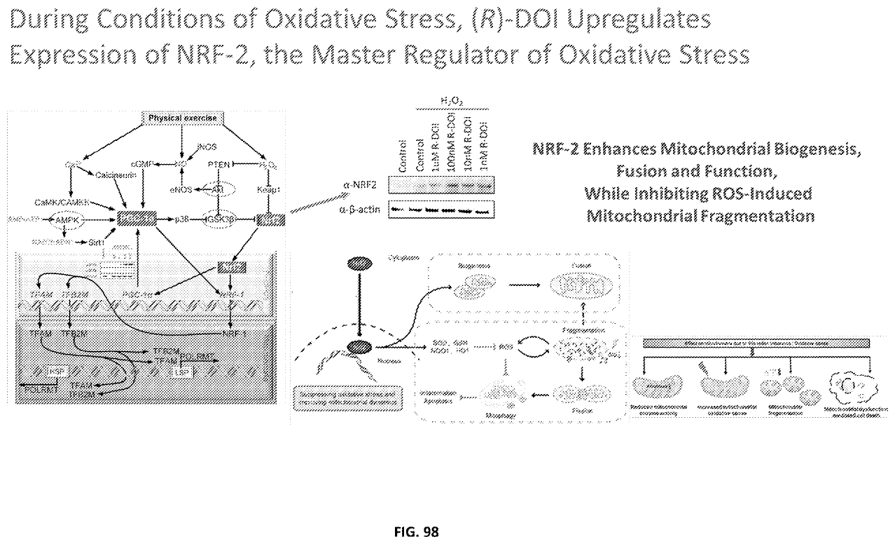

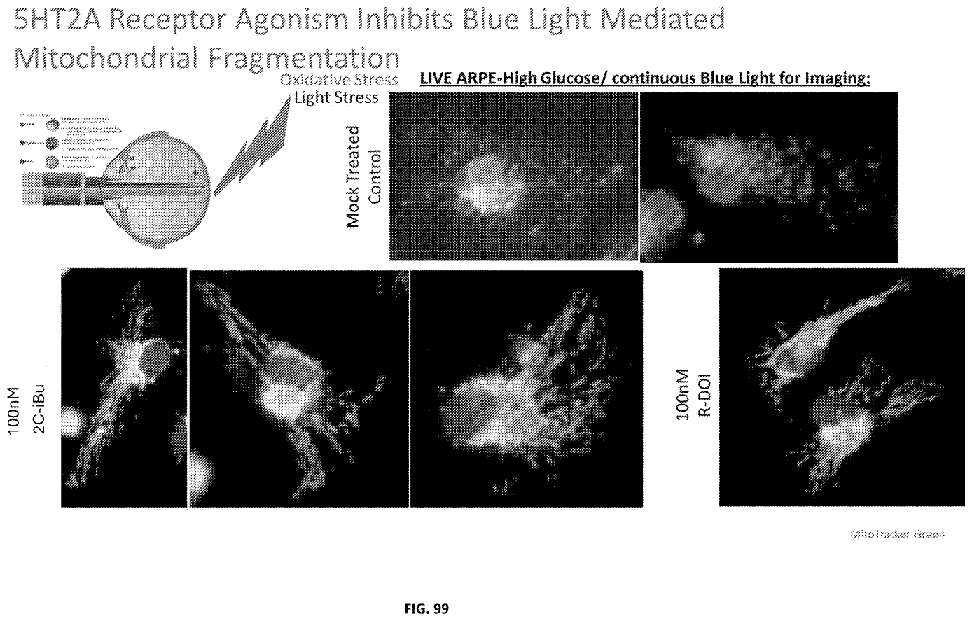

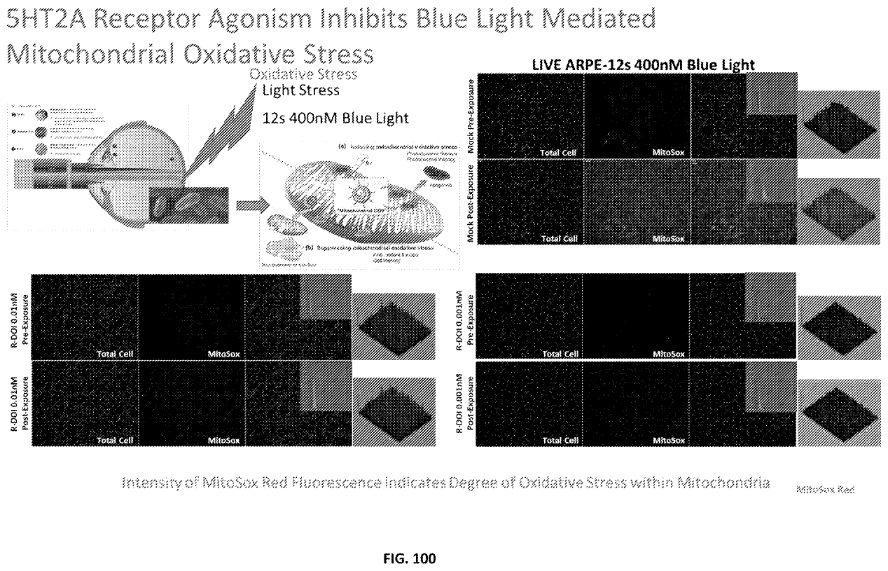

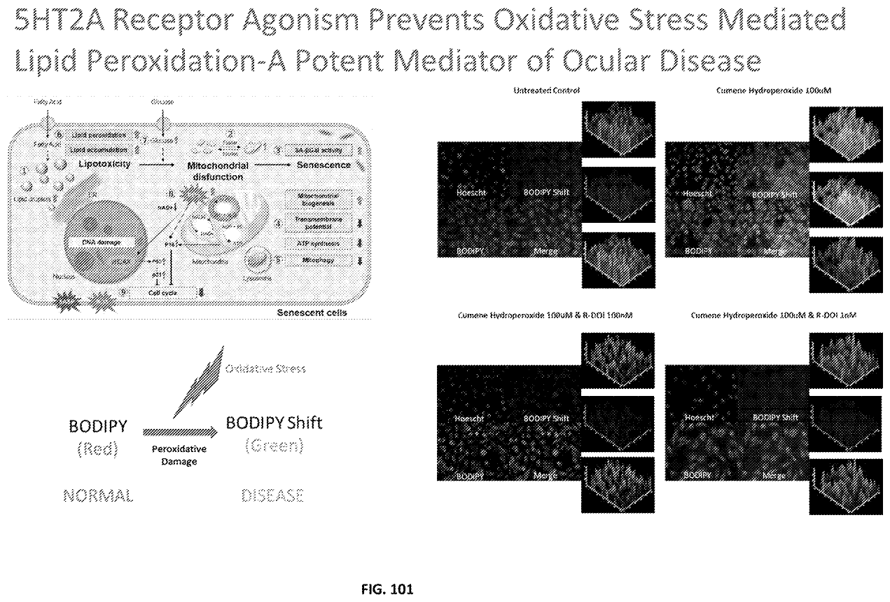

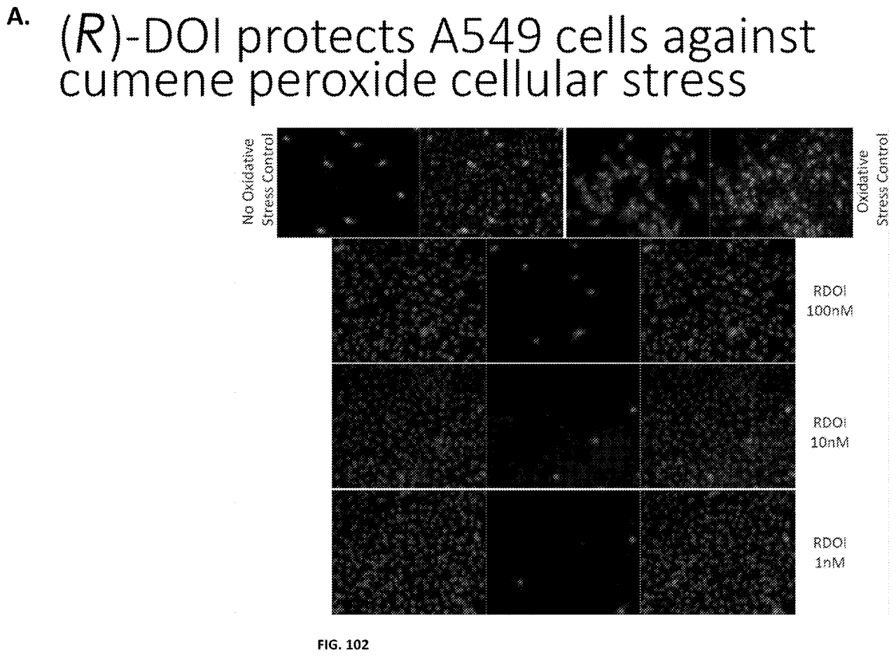

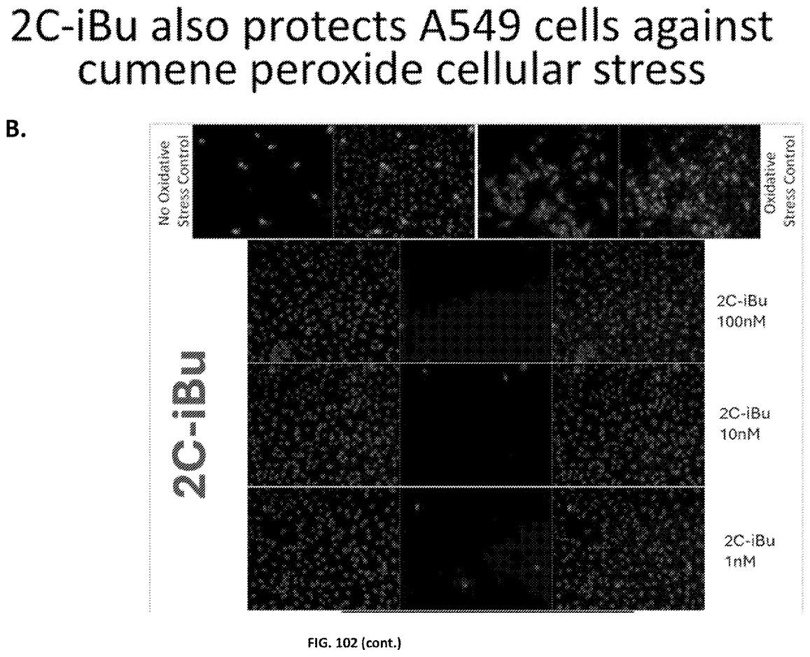

5-HT2A Receptor KNOCKDOWNS DECREASE Mitochondrial Function And ATP Production (FIG. 97).
During Conditions of Oxidative Stress, (R)-DOI Upregulates Expression of NRF-2, the Master Regulator of Oxidative Stress (FIG. 98).
5HT2A Receptor Agonism Inhibits Blue Light Mediated Mitochondrial Fragmentation (FIG. 99).
5HT2A Receptor Agonism Inhibits Blue Light Mediated Mitochondrial Oxidative Stress (FIG. 100).
5HT2A Receptor Agonism Prevents Oxidative Stress Mediated Lipid Peroxidation—A Potent Mediator of Ocular Disease (FIG. 101).
Oxidative Stress Mediated Cell Death (FIG. 102).

### EQUIVALENTS

Those skilled in the art will recognize, or be able to ascertain, using no more than routine experimentation, numerous equivalents to the specific substances and procedures described herein. Such equivalents are considered to be within the scope of this invention and are covered by the following claims.

What is claimed is:

1. A method of treating or preventing senescence, mitochondrial dysfunction, or a disease or disorder thereof, the method comprising administering a therapeutically effective amount of a 5-HT_{2A} receptor agonist to a subject in need thereof, wherein the 5-HT2A receptor agonist is selected from the group consisting of:

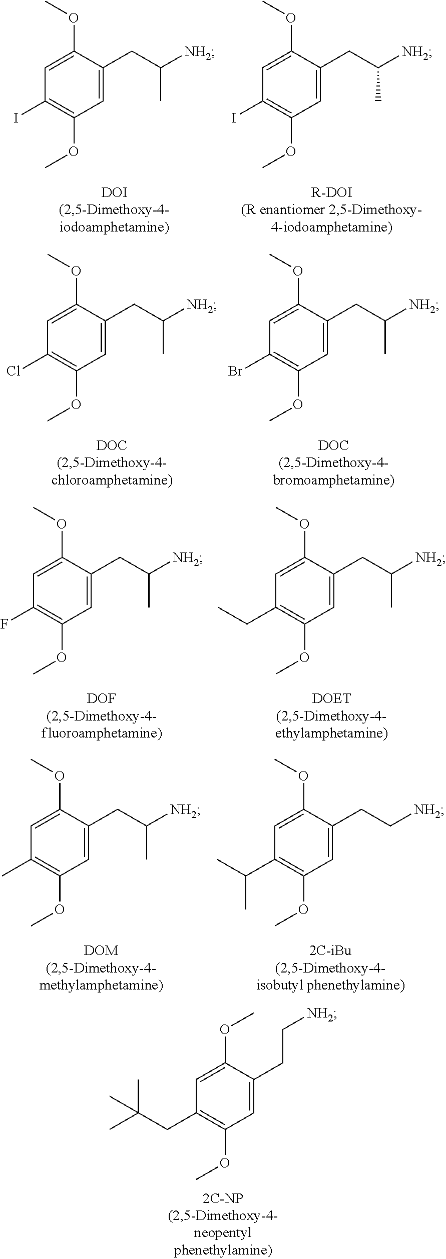

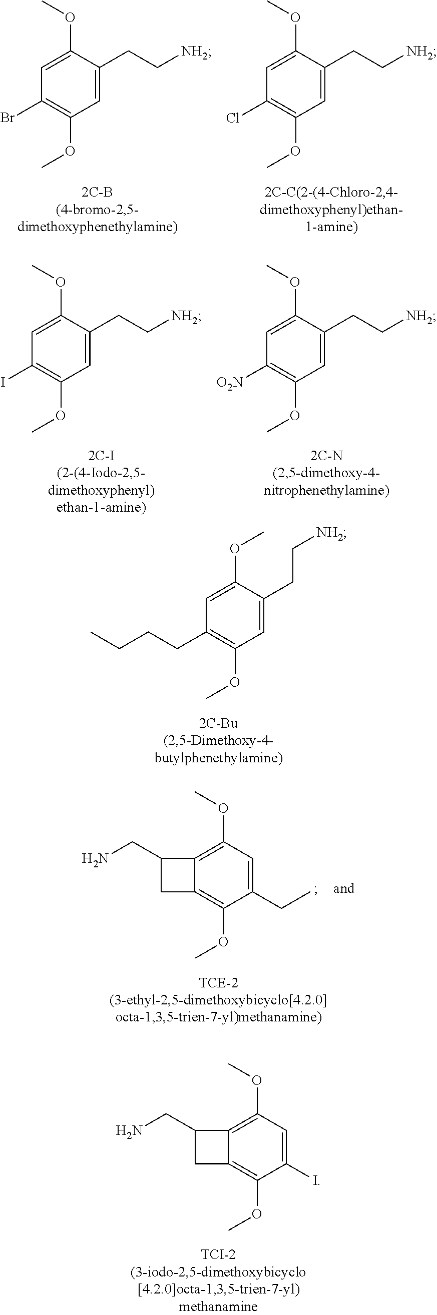

2. The method of claim 1, wherein the disease or disorder comprises a neurodegenerative disease, a metabolic disorder, an inflammatory or autoimmune disease, or an aging related disorder.

3. The method of claim 2, wherein the neurodegenerative disease comprises dementia, Alzheimer's disease, Parkinson's disease, Huntington's disease, dementia, cognitive impairment, and motor neuron dysfunction.

4. The method of claim 2, wherein the metabolic disorder comprises type II diabetes, obesity, hyperglycemia, glucose intolerance, insulin resistance (i.e., hyperinsulinemia, metabolic syndrome, syndrome X), hypercholesterolemia, hypertension, hyperlipoproteinemia, hyperlipidemia (e.g., dyslipidemia), hypertriglylceridemia, cardiovascular disease, atherosclerosis, peripheral vascular disease, kidney disease, ketoacidosis, thrombotic disorders, nephropathy, diabetic neuropathy, diabetic retinopathy, sexual dysfunction, dermatopathy, dyspepsia, hypoglycemia, cancer or edema.

5. The method of claim 2, wherein the inflammatory or autoimmune disease comprises celiac disease, vasculitis, lupus, chronic obstructive pulmonary disease (COPD), atherosclerosis, arthritis, psoriasis, atopic dermatitis, vitiligo, keratitis, allergic conjunctivitis, uveitis, retinitis, osteoarthritis, rheumatoid arthritis, osteoporosis, oral mucositis, inflammatory bowel disease, kyphosis, and herniated intervertebral disc.

6. The method of claim 2, wherein the aging related disorder comprises renal disease, renal failure, frailty, hearing loss, muscle fatigue, skin conditions, skin wound healing, liver fibrosis, pancreatic fibrosis, oral submucosa fibrosis, sarcopenia, cancer, dementia, cardiovascular disease, such as atherosclerosis, hypertension, diabetes mellitus (type I or type II) arthritis, dermatitis, cataracts, Alzheimer's disease, Parkinson's disease, diabetic retinopathies, retinopathies, glaucoma, age-related macular degeneration (AMD), and osteoporosis.

7. The method of claim 1, wherein the therapeutically effective amount comprises greater than about 1 uM, about 1 uM, less than about 1 uM, about 100 nM, about 10 nM, about 1 nM, about 0.1 nM, about 0.05 nM, about 0.001, about 0.0001 nM, about 0.00001 nM, and less than 0.00001 nM.

8. The method of claim 1, wherein the therapeutically effective amount of R-DOI is about 10 nM to less about 0.001 nM.

9. The method of claim 1, wherein the therapeutically effective amount of TCE-2 is about 100 nM.

10. The method of claim 1, wherein the therapeutically effective amount of TCI-2 is about 1 uM, less than about 1 uM, about 100 nM, about 10 nM, about 1 nM, about 0.1 nM, about 0.05 nM, about 0.001, and less than 0.001 nM.

11. The method of claim 1, wherein the therapeutically effective amount of 2C-NP is about 0.1 nM, about 0.01 nM, about 0.001 nM, about 0.0001 nM, or less than 0.1 nM.

12. The method of claim 1, wherein the therapeutically effective amount of 2C-B, 2C-C, 2C-I, 2C-Bu, or 2C-N is about 0.1 nM, about 0.001 nM, about 0.0001 nM, or less than 0.1 nM.

13. The method of claim 1, wherein the therapeutically effective amount of DOI is about 10 uM to about 50 uM.

14. The method of claim 1, wherein the 5-HT_{2A} receptor agonist is formulated as a pharmaceutical composition comprising one or more pharmaceutically acceptable excipients.

15. The method of claim 14, wherein the pharmaceutical composition is formulated for topical administration, ocular administration, parenteral administration, intravenous administration, intranasal administration, or oral administration.

16. The method of claim 15, wherein the pharmaceutical composition is formulated for ocular administration can comprise an eye drop, a contact lens, an ocular hydrogel, an intraocular implant, or intravitreal injection.

17. A method of identifying a subject responsive to a 5-HT_{2A} receptor agonist of claim 1 for the treatment or prevention of senescence, mitochondrial dysfunction, or a disease or disorder thereof, the method comprising determining if the subject meets one or more of the following criteria:
i. at or above the transition age of ocular disease development:
ii. a blood glucose level greater than or equal to about 6 mmol/L;
iii. presents with lipofuscin and drusen deposition;
iv. presents with autofluorescent oxidized flavoproteins;
v. an Early Treatment Diabetic Retinopathy Study (ETDRS) disease severity score of about 20 or greater; and/or
vi. presents with a retinopathy.

18. The method of claim 17, wherein the transition age of ocular disease development comprises about 50 years old to about 60 years old in a human subject.
